(19) United States
(12) Patent Application Publication
(10) Pub. No.: US 2026/0217842 A1
(43) Pub. Date: Jul. 30, 2026

(54) CD40L-SPECIFIC TN3-DERIVED SCAFFOLDS FOR THE TREATMENT AND PREVENTION OF SJÖGREN'S SYNDROME

(71) Applicant: Viela Bio, Inc., Deerfield, IL (US)

(72) Inventors: Ilias ALEVIZOS ALEVIZOS, Washington, DC (US); William A. REES, North Bethesda, MD (US); Claire L. EMSON, Poolesville, MD (US); Michael SMITH, Magnolia, DE (US); Tuyet-Hang T. PHAM, Columbia, MD (US)

(73) Assignee: Viela Bio, Inc., Deerfield, IL (US)

(21) Appl. No.: 19/146,752

(22) PCT Filed: Jan. 12, 2024
(86) PCT No.: PCT/US24/11477

§ 371 Date: Jul. 9, 2025

Related U.S. Application Data
(60) Provisional application No. 63/479,830, filed on Jan. 13, 2023.
(60) Provisional application No. 63/597,860, filed on Nov. 10, 2023.

(51) Int. Cl. C07K 16/28; A61K 47/64; A61P 1/00

(52) U.S. Cl. CPC C07K 16/2875; A61K 47/64; A61K 47/643; A61P 1/00; C07K 2318/20

(57) ABSTRACT

Provided are compositions and methods comprising a CD40L-specific Tn3 scaffold. Also provided are methods of utilizing the same for the prevention and treatment of Sjögren's Syndrome.

### CROSS-REFERENCE TO RELATED APPLICATIONS

This application claims priority to U.S. Application No. 63/597,860, filed on Nov. 10, 2023, and U.S. Application No. 63/479,830, filed on Jan. 13, 2023, which are hereby incorporated by reference in their entireties for all purposes.

### REFERENCE TO AN ELECTRONIC SEQUENCE LISTING

The contents of the electronic sequence listing (HOPA_065_02WO_SeqList_ST26.xml; Size: 24,509 bytes; and Date of Creation: Jan. 12, 2024) are herein incorporated by reference in its entirety.

### FIELD OF THE DISCLOSURE

The present disclosure is related to compositions comprising a Tn3 scaffold and methods using the same in the treatment and prevention of Sjögren's syndrome. Also provided are methods of using the same in the treatment of rheumatoid arthritis.

### BACKGROUND

Sjögren's syndrome (SS) is a systemic autoimmune disease characterized by chronic lymphocytic inflammation of the exocrine glands, mainly the salivary and lacrimal glands, leading to loss of function manifesting as excessive dryness. Additionally, extra glandular manifestations are described as multi-organ involvement affecting musculoskeletal, pulmonary, renal, nervous, dermatological, gastrointestinal, hematological, hepatobiliary, or vascular systems, while fatigue is one of the most prominent comorbidities. The joints, lung, skin, and peripheral nerves are the most frequent organ systems involved, while cytopenia, hypocomplementemia, and cryoglobulinemia at diagnosis are strongly associated with greater systemic activity.

The subjective aspects of SS, which include the patient's perception of dryness, musculoskeletal pain, and fatigue can be debilitating and have been shown to have a substantial negative impact on quality of life (QoL). The major contributors to the decreased QoL are dryness and fatigue. QoL is also affected by psychological and emotional challenges and impaired social life with dependency on relatives in daily life and difficulties at work, as well as with other tasks. SS patients with exocrine dysfunction, severe subjective symptoms defined by an European League Against Rheumatism (EULAR) Sjögren's Syndrome Patient Reported Index (ESSPRI) score of ≥5, considered as the cut-off point for “unsatisfactory symptom state” but with lower systemic disease activity (with EULAR) Sjögren's Syndrome Disease Activity Index (ESSDAI) score<5] represents a large subset of SS patients who have been excluded from recent trials despite the substantial disease burden and overall unacceptable health status.

About 15-20% of SS patients present with systemic manifestations beyond the commonly affected exocrine glands (salivary and ocular). Moderate to high systemic activity in SS patients can be defined by ESSDAI≥5. These manifestations most commonly include arthritis, lung disease, renal disease, vasculitis, neuropathies, and autonomic nervous system dysfunction, which accompany glandular involvement. Moderate to high disease activity is not only debilitating but can also lead to dysfunction of the affected organ(s), or to hematologic pathologies, including thrombocytopenia and lymphoma, that have been associated with increased risk of mortality. No biologics or disease-modifying anti-rheumatic drugs (DMARDs) have been shown to be efficacious in significantly reducing SS systemic disease activity. Currently, there are no approved immunomodulating agents or evidence-based therapeutic guidelines available for treatment of the extra glandular manifestations of SS. Therefore, standard of care for extra glandular manifestations varies widely and is based on local practices, expert opinion, and personal experience of the treating physicians.

There is a need for new treatments aimed at to reduce the substantial disease burden and overall unacceptable health status.

### BRIEF SUMMARY

Provided herein methods for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the subject in need has increased B cells of subsets selected from the group consisting of: Ki67+CD27+ memory cells, CD27 high CD38 high plasmablasts, and CD11c high atypical memory cells, as compared to a baseline level of the subject or a level in an untreated subject. In aspects, at least one CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21. In aspects, the subject has a European League Against Rheumatism (EULAR) Sjögren's Syndrome Disease Activity Index (ESSDAI) score≥5. In aspects, the ESSDAI score is assessed based on the ESSDAI domains consisting of cutaneous, renal, articular, muscular, hematological, glandular, constitutional, lymphadenopathic, and biological. In aspects, the subject has (a) a ESSDAI score<5, (b) a EULAR Sjögren's Syndrome Patient Reported Index (ESSPRI) score≥5, and (c) whole stimulated salivary flow>0.1 mL/min. In aspects, the Tn3 scaffold is administered as an induction dose and as a maintenance dose thereafter. In aspects, the induction dose comprises administering the Tn3 scaffold once about every 2 weeks for at least 3 doses. In aspects, the maintenance dose comprises administering the Tn3 scaffold once about every 4 weeks for at least 4 doses. In aspects, the time between the last induction dose and the first maintenance dose is about 4 weeks. In aspects, the Tn3 scaffold is administered once about every 4 weeks, once about every 2 months, once about every 3 months, once about every 4 months, or once about every 6 months. In aspects, the Tn3 scaffold is administered for at least 4 doses. In aspects, the Tn3 scaffold is administered for at least 5 doses. In aspects, the Tn3 scaffold is administered intravenously, subcutaneously, orally, intramuscularly, intrathecally, sublingually, rectally, vaginally, cutaneously, systemically, topically, transdermally, or by way of inhalation. In aspects, the Tn3 scaffold is administered intravenously. In aspects, the Tn3 scaffold comprises two CD40L-specific monomer subunits connected in tandem. In aspects, the two CD40L-specific monomer subunits each comprise SEQ ID NO: 3. In aspects, the CD40L-specific monomer subunits are connected by a linker. In aspects, at least one CD40L-specific monomer subunit is fused or conjugated to a polyethylene glycol (PEG) directly. In aspects, at least one CD40L-specific monomer subunit is fused or conjugated to a polyethylene glycol (PEG) via a linker. In aspects, the linker comprises a peptide linker. In aspects, the linker comprises SEQ ID NO: 7, SEQ ID NO: 8, SEQ ID NO: 9, or SEQ ID NO: 10. In aspects, at least one CD40L-specific monomer subunit is fused or conjugated to an albumin. In aspects, the albumin is human serum albumin (HSA). In aspects, the HSA is a variant HSA comprising SEQ ID NO: 4. In aspects, the Tn3 scaffold comprising the CD40L-specific monomer subunit is administered at a dose of about 1500 mg, about 2500 mg, or about 3000 mg. In aspects, the dose is about 1500 mg. In aspects, the dose is about 2500 mg. In aspects, the dose is about 3000 mg.

Provided are methods of treating Sjögren's syndrome (SS) in a subject in need thereof comprising: administering a Tn3 scaffold at a dose of about 1500 mg, about 2500 mg, or about 3000 mg to the subject, wherein the Tn3 scaffold comprises SEQ ID NO: 1, and wherein the subject in need has increased B cells of subsets selected from the group consisting of: Ki67+CD27+ memory cells, CD27 high CD38 high plasmablasts, and CD11c high atypical memory cells, as compared to an otherwise healthy subject. In aspects, the Tn3 scaffold is administered as an induction dose and as one or more maintenance doses thereafter. In aspects, the induction dose and the maintenance doses are the same amount. In aspects, the induction dose and the maintenance doses are different amounts. In aspects, at least one dose is 3000 mg. In aspects, the induction dose and at least one maintenance dose are administered about 1 month apart, about 2 months apart, about 3 months apart, about 4 months, or about 6 months apart. In aspects, the induction dose comprises administering the Tn3 scaffold about every 2 weeks for at least 3 doses and the maintenance dose comprises administering the Tn3 scaffold once about every 4 weeks for at least 4 doses. In aspects, the Tn3 scaffold comprises SEQ ID NO: 1. In aspects, the administration is effective in reducing a ESSDAI score as compared to an otherwise comparable subject receiving an administration of placebo control. In aspects, the reduction is of at least about 1 point, 2 points, 3 points, 4 points, or 5 points. In aspects, the reduction is of at least about 6 points. In aspects, the administration is effective in reducing levels of B cells as compared to the baseline level of the subject or the level in the untreated subject. In aspects, the administration is effective in reducing a level of serum CXCL13 as compared to a baseline level in the subject or a level in an untreated subject. In aspects, the administration is effective in reducing rheumatoid factor (RF) autoantibodies as compared to a baseline level in the subject or a level in an untreated subject. In aspects, the reducing is detected at least by day 14 following the administering.

Provided are methods of treating autoimmune disease in a subject in need thereof comprising administering a Tn3 scaffold comprising a CD40L-specific monomer subunit that binds CD40L to the subject when a level of a biomarker is increased in a sample of the subject as compared to a healthy baseline level, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16. In aspects, the biomarker is expressed on the surface of a cell. In aspects, the biomarker is an antibody. In aspects, the biomarker is secreted by a cell. In aspects, the biomarker is selected from the group consisting of: Ki67+CD27+ memory cell, CD27 high CD38 high plasmablast, CD11c high atypical memory cell, CXCL13, RF autoantibody, and any combination thereof. In aspects, the biomarker is a protein. In aspects, the protein biomarker is selected from the group consisting of: TNFRSF13B, BAFF, CXCL10, CXCL12, TL6, CCL21, CXCL9, IL12B, TNFRSF4, LAMP3, CD22, CD79B, SLAMF7, IL10, TNFRSF8, CXCL11, CCL19, TNFRSF9, TNFRSF6B, NOS3, CD72, ADA2, TL2RA, CD27, PDCD1, IL12A, IL12B, LAG3, ADGRE1, TIGIT, FCAMR, CD80, and any combination thereof. In aspects, the autoimmune disease is selected from the group consisting of: systemic lupus erythematosus (SLE), lupus nephritis, cutaneous lupus erythematosus (CLE), Sjögren's syndrome, dermatomyositis, myositis, sclerosis, diabetes, Hashimoto's disease, autoimmune adrenal insufficiency, anemia, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy. In aspects, the arthritis is rheumatoid arthritis. In aspects, the administering is continued until the level of the biomarker reverts to the healthy baseline. In aspects, the level of the biomarker is analyzed routinely. In aspects, the Tn3 scaffold comprises SEQ ID NO: 1. In aspects, the Tn3 scaffold is administered at a dose of about 1500 mg, about 2500 mg, or about 3000 mg.

Provided are methods of treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the treatment of the subject with the Tn3 scaffold decreases the level of B cells selected from the group consisting of: Ki67+CD27+ memory cells, CD27 high CD38 high plasmablasts, and CD11c high atypical memory cells, as compared to a baseline level of the subject or a level in an untreated subject. In aspects, the Tn3 scaffold comprises SEQ ID NO: 1. In aspects, the Tn3 scaffold is administered at a dose of about 1500 mg, about 2500 mg, or about 3000 mg. In aspects, the level of B cells is reduced by at least about 1-fold, 5-fold, 10-fold, 30-fold, 50-fold, 75-fold, 100-fold, 150-fold, or 200-fold.

Provided are methods of treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the treatment of the subject with the Tn3 scaffold decreases the level of T cells selected from the group consisting of: Ki67+/CD3+, Ki67+/CD4+ cells, Ki67+/CD3+/CD4+ cells, Ki67+/CD3+/CD4+/CD45RA−/CCRT7+ cells, Ki67+/CD3+/CD4+/CD45RA−/CCR7− cells, Ki67+/CD3+/CD4+/CXCR5+/ICOS+ cells, Ki67+/CD3+/CD8+ cells, Ki67+/CD3+/CD8+/CD45RA−/CCR7+ cells, Ki67+/CD3+/CD8+/CD45RA/CCR7− cells, and Ki67+/CD3+/CD8+/CXCR5+ cells, and CXCR5+/ICOS+/CD3+/CD4+ cells, as compared to a baseline level of the subject or a level in an untreated subject. In aspects, the Tn3 scaffold comprises SEQ ID NO: 1. In aspects, the Tn3 scaffold is administered at a dose of about 1500 mg, about 2500 mg, or about 3000 mg. In aspects, the level of T cells is reduced by at least about 1-fold, 5-fold, 10-fold, 30-fold, 50-fold, 75-fold, 100-fold, 150-fold, or 200-fold.

### BRIEF DESCRIPTION OF THE DRAWINGS

FIG. 1 shows an exemplary study flow diagram comprising adults with moderate to severe (moderate to high) systemic disease activity defined by ESSDAI≥5, and presence of anti-Ro autoantibodies and/or rheumatoid factor (RF). Seventy four subjects were randomized (1:1) to receive VIB4920 1500 mg or placebo intravenously once every 2 weeks×3 doses, then once every 4 weeks (Q4W) for 4 additional doses (Stage I). Starting on day 169, subjects randomized to VIB4920 received placebo Q4W for 5 doses and subjects randomized to placebo received VIB4920 Q4W for 5 doses (Stage II). Randomization was stratified by EULAR Sjögren's Syndrome Disease Activity Index (ESSDAI) score at screening (<10 points vs≥10 points). Δ=delta; D=days; EoS=End of Study; EP=endpoint; ESSDAI=European League Against Rheumatism Sjögren's Syndrome Disease Activity Index; ESSPRI=in EULAR Sjögren's Syndrome Patient Reported Index; IA=interim analysis; mos.=months; PE=primary endpoint.

FIG. 2 depicts a heat mat showing the impact of dazodalibep on: top, disease activity scores, tender and swollen joint counts, and blood biomarkers of T and B cell costimulation in patients with moderate-to-severe active rheumatoid arthritis; middle, CXCL13 and Rheumatoid Factor, and; bottom, PS mem B cells, Plasmablasts, precursor memory b cells, T cells, and Tfh cells. DAZ, Dazodalibep; FC, fold change; DAS28-CRP, disease activity score-28 with c-reactive protein; SDAI, simplified disease activity index; CDAI, clinical disease activity index; PS Mem B Cells, post-switched memory B cells; Tfh Cells, T follicular helper cells.

FIG. 3A to FIG. 3G show that subjects in both population 1 and 2 dosed with VIB4920 exhibited reduced CXCL13 (FIG. 3A), rheumatoid factor (FIG. 3B), post-switched memory B cells (FIG. 3C), plasmablasts (FIG. 3D), precursor memory B cells (FIG. 3E), T cells (FIG. 3F), and T follicular helper cells (FIG. 3G) levels. Data were analyzed using Mann-Whitney U test on fold-change of baseline values; *p<0.05; **p<0.01, ***p<0.001; DAZ, Dazodalibep (VIB4920); DAZ-PBO, subjects received DAZ in Stage I and PBO in Stage II; PBO, placebo; FC, fold-change; PBO-DAZ, subjects received PBO in Stage I and DAZ in Stage II; RF subset only included those subjects who were RF positive at baseline.

FIG. 4 is a heatmap that shows that subjects in both population 1 and 2 dosed with VIB4920 exhibited various reduced T and B cell biomarkers.

FIG. 5 depicts a heat map showing the impact of dazodalibep (DAZ) versus placebo (PBO) on population 1 (Pop 1) and population 2 (Pop 2) at baseline, Day 15, and Day 169 post-administration as compared to a healthy, untreated cohort. The levels of 29 biomarkers were quantified as FC to baseline. FC=fold-change.

FIGS. 6A-6DDD depict graphs of biomarker levels for PDCD1 (FIGS. 6A-6B), TNFRSF8 (FIGS. 6C-6D), CD22 (FIGS. 6E-6F), IL12B (FIGS. 6G-6H), TNFRSF9 (FIGS. 6I-6J), CXCL13 (FIGS. 6K-6L), TNFRSF4 (FIGS. 6M-6N), IL2RA (FIGS. 6O-6P), TIGIT (FIGS. 6Q-6R), IL2A/IL2B (FIGS. 6S-6T), CCL19 (FIGS. 6U-6V), NOS3 (FIGS. 6W-6X), CD80 (FIGS. 6Y-6Z), CD72 (FIGS. 6AA-6BB), FCAMR (FIGS. 6CC-6DD), TNFRSF6B (FIGS. 6EE-6FF), CD79B (FIGS. 6GG-6HH), CXCL9 (FIGS. 6II-6JJ), CXCL11 (FIGS. 6KK-6LL), ADGRE1 (FIGS. 6MM-6NN), SLAMF7 (FIGS. 6OO-6PP), CD27 (FIGS. 6QQ-6RR), LAG3 (FIGS. 6SS-6TT), CXCL10 (FIGS. 6UU-6VV), ADA2 (FIGS. 6WW-6XX), IL10 (FIGS. 6YY-6ZZ), CCL21 (FIGS. 6AAA-6BBB), and TNFRSF13B (FIGS. 6CCC-6DDD). Levels depicted as absolute value of normalized protein expression (NPX) and fold-change level from baseline. Data points taken at Day 1, Day 15, Day 169, and Day 309 post-administration. *=p≤0.05; **=p≤0.01; ***=p≤0.001.

### DETAILED DESCRIPTION

Provided herein are Tn3 scaffolds that are anti-cluster of differentiation (CD) 40 ligand (CD40L)-third fibronectin type III (Fn3) protein domain of human Tenascin C (Tn3) protein fusion proteins and methods of using the same in SS. In aspects, compositions and methods provided are utilized for the treatment of glandular and extra glandular manifestations of SS patients with moderate to high systemic disease activity and for the treatment of subjective complaints of dryness, fatigue, and pain of SS patients with exocrine dysfunction. Activation of cluster of differentiation (CD40) has been shown to be critical in germinal center formation, immunoglobulin (Ig)-class switching and expression of cytokines, such as interferon-α, tumor necrosis factor-α, and interleukin-6, all of which have been previously associated with the pathophysiology of SS. Dysregulation of the CD40/CD40L has been observed in patients with SS in both the circulating cells and in the epithelial salivary cells. These observations suggest that inhibition of the CD40L/CD40 pathway may be beneficial in SS.

The following description includes information that may be useful in understanding the present disclosure. It is not an admission that any of the information provided herein is prior art or relevant to the presently claimed disclosures, or that any publication specifically or implicitly referenced is prior art.

### Definitions

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques and/or substitutions of equivalent techniques that would be apparent to one of skill in the art.

As used herein, the singular forms “a,” “an,” and “the” include plural referents unless the content clearly dictates otherwise.

The term “about” or “approximately” when immediately preceding a numerical value means a range (e.g., plus or minus 10% of that value). For example, “about 50” can mean 45 to 55, “about 25,000” can mean 22,500 to 27,500, etc., unless the context of the disclosure indicates otherwise, or is inconsistent with such an interpretation. For example, in a list of numerical values such as “about 49, about 50, about 55, . . . ”, “about 50” means a range extending to less than half the interval(s) between the preceding and subsequent values, e.g., more than 49.5 to less than 52.5. Furthermore, the phrases “less than about” a value or “greater than about” a value should be understood in view of the definition of the term “about” provided herein. Similarly, the term “about” when preceding a series of numerical values or a range of values (e.g., “about 10, 20, 30” or “about 10-30”) refers, respectively to all values in the series, or the endpoints of the range.

As used herein, the term “subject” refers to any individual, e.g., a human or a non-human mammal, for whom diagnosis, prognosis, or therapy is desired. The term “subject” may mean a human or non-human mammal affected, likely to be affected, or suspected to be affected with a disease. The terms “subject” and “patient” are used interchangeably herein. In aspects, the subject is a mammal. A mammal includes primates, such as humans, monkeys, chimpanzee, and apes, and non-primates such as domestic animals, including laboratory animals (such as rabbits and rodents, e.g., guinea pig, rat, or mouse) and household pets and farm animals (e.g., cats, dogs, swine, cattle, sheep, goats, horses, rabbits), and non-domestic animals, such as wildlife, birds, reptile; fish, or the like. Typically, the subject is a human subject.

As used herein, the term “a subject in need thereof” includes subjects that could or would benefit from the methods described herein. Subjects in need of treatment include, without limitation, those already with the condition or disorder, those prone to having the condition or disorder, those in which the condition or disorder is suspected, as well as those in which the condition or disorder is to be prevented, ameliorated, or reversed.

As used herein, “treating” or “treat” describes the management and care of a subject for the purpose of combating a disease, condition, or disorder and includes the administration of Tn3 scaffolds used in the methods described herein to alleviate the symptoms or complications of a disease, condition or disorder, or to eliminate the disease, condition or disorder. Thus, the term “treat” or “treating” refers to both therapeutic measures and prophylactic or preventative measures, wherein the objective is to prevent, slow down (lessen), or ameliorate the progression of a disease (e.g., SS). Beneficial or desired clinical results include, but are not limited to, alleviation of symptoms, diminishing the extent of the disease, stabilized (i.e., not worsening) state of the disease, delaying or slowing of disease progression, amelioration or palliation of the disease state, and reversing the disease (whether partial or total). The term “treat” can also include treatment of a cell in vitro or an animal model.

As used herein, “fused” refers to at least two polypeptides joined recombinantly. As used herein “conjugated” refers to formation of a bond between two components by chemical reaction. The bond may be covalent or non-covalent. Typically, two components that are conjugated to each other are chemically connected via a covalent bond.

When referring to a nucleic acid sequence or protein sequence, the term “identity” is used to denote similarity between two sequences. Unless otherwise indicated, percent identities described herein are determined using the BLAST algorithm available at the world wide web address: blast.ncbi.nlm.nih.gov/Blast.cgi using default parameters.

Described herein are methods for treating SS using a Tn3 scaffold comprising a CD40L-specific monomer subunit.

In aspects, the Tn3 scaffold is used in methods of treating SS. SS is a systemic autoimmune disease characterized by chronic lymphocytic inflammation of the exocrine glands, mainly the salivary and lacrimal glands, leading to loss of function manifesting as excessive dryness. Extraglandular manifestations are described as multi-organ involvement affecting musculoskeletal, pulmonary, renal, nervous, dermatological, gastrointestinal, hematological, hepatobiliary, or vascular systems, while fatigue is one of the most prominent comorbidities. SS may also present in association with other autoimmune diseases. In aspects, the methods comprise treating patients with SS with moderate to high systemic disease activity by administering the Tn3 scaffold. In aspects, the moderate to high systemic disease activity is defined by European League Against Rheumatism (EULAR) Sjögren's Syndrome Disease Activity Index (ESSDAI)≥5. In aspects, the methods comprise treating patients with SS with moderate to high (in some cases severe) subjective symptoms by administering the Tn3 scaffold. In aspects, the moderate to high subjective symptoms are defined by EULAR Sjögren's Syndrome Patient Reported Index (ESSPRI) score≥5 and residual stimulated salivary flow but with mild systemic disease activity defined by ESSDAI score<5.

In aspects, the methods comprise treating SS in a subject in need thereof by thereof by administering the Tn3 scaffold comprising a CD40L-specific monomer subunit. In aspects, the Tn3 scaffold binds to CD40L. In aspects, the monomer subunit of the Tn3 scaffold comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16. In aspects, the Tn3 scaffold is VIB4920. The CD40 receptor is a member of the TNF family of receptors expressed on the plasma membrane of antigen-stimulated B cells, macrophage, and dendritic cells. The CD40 receptor functions to provide a co-stimulatory signal for B cells that have bound antigen. The cognate ligand for CD40 is CD40L (also known as CD154), which is expressed on the plasma membrane of T cells and other cell types, including platelets.

### Tn3 Scaffolds

Provided herein are compositions that bind CD40L. In aspects, provided compositions comprise CD40L antagonists. In aspects, provided herein are compositions that comprise a Tn3 scaffold comprising a CD40L-specific monomer subunit (e.g., “Tn3 scaffold”). In aspects, provided herein are compositions that comprise a Tn3 scaffold comprising two CD40L-specific monomer subunits.

In aspects, provided compositions may comprise the amino acid sequence as described in Int'l Appl. Nos. PCT/US2012/059477 and PCT/US2019/052997, which are incorporated herein by reference in their entireties. In aspects, provided compositions may comprise the amino acid sequence as shown in SEQ ID NO: 1 (referred to herein as VIB4920). VIB4920 comprises a bivalent CD40L-specific Tn3 protein fused to a has protein.

In aspects, a CD40L monomer subunit of a Tn3 scaffold comprises seven beta strands designated A, B, C, D, E, F, and G, and six loop regions designated AB, BC, CD, DE, EF, and FG. In aspects, the Tn3 scaffold comprises a single CD40L-specific monomer subunit. In aspects, the Tn3 scaffold comprises two CD40L-specific monomer subunits. In aspects, the two CD40L-specific monomer subunits are connected in tandem. In aspects, the two CD40L-specific monomer subunits are connected by a linker. In aspects, the linker comprises a peptide linker, which can be a flexible peptide linker. In aspects, the peptide linker comprises a (GₘX)ₙ sequence wherein X is Serine (S), Alanine (A), Glycine (G), Leu (L), Isoleucine (I), or Valine (V); m and n are integer values; m is 1, 2, 3 or 4; and n is 1, 2, 3, 4, 5, 6, or 7.

In aspects, the Tn3 scaffold comprises a linker which comprises a functional moiety. In aspects, this functional moiety is an immunoglobulin or a fragment thereof. In aspects, this immunoglobulin or fragment thereof comprises an Fc domain. In aspects, this Fc domain fails to induce at least one FcγR-mediated effector function (e.g., Fc-deficient). In aspects, this at least one FcγR-mediated effector function is antibody-dependent cellular cytotoxicity (ADCC).

In aspects, the Tn3 scaffold comprises seven beta strands designated A, B, C, D, E, F, and G, and six loop regions designated AB, BC, CD, DE, EF, and FG, wherein the AB loop comprises or consists of SEQ ID NO: 11, the BC loop comprises or consists of SEQ ID NO: 12, the CD loop comprises or consists of SEQ ID NO: 13, the DE loop comprises or consists of SEQ ID NO: 14, the EF loop comprises or consists of SEQ ID NO: 15, and the FG loop comprises or consists of SEQ ID NO: 16. In aspects, the Tn3 scaffold comprises or consists of SEQ ID NO: 1 (also known as VIB4920). In aspects, beta strand A comprises or consists of SEQ ID NO: 5, beta strand B comprises or consists of SEQ ID NO: 6, beta strand C comprises or consists of SEQ ID NO: 17, beta strand D comprises or consists of SEQ ID NO: 18, beta strand E comprises or consists of SEQ ID NO: 19, beta strand F comprises or consists of SEQ ID NO: 20, and beta strand G comprises or consists of SEQ ID NO: 21.

In aspects, one or more CD40L-specific Tn3 monomers have a beta strand A comprising or consisting of IEV (SEQ ID NO: 5), RLDAPSQIEV (SEQ ID NO: 23), or SQIEV (SEQ ID NO: 24). In aspects, a Tn3 scaffold may comprise one or more CD40L-specific Tn3 monomers having the same or different beta strand A sequences. For example, a first CD40L-specific Tn3 monomer beta strand A may comprise or consist of IEV (SEQ ID NO: 5) and a second CD40L-specific Tn3 monomer beta strand A may comprise or consist of RLDAPSQIEV (SEQ ID NO: 23) or SQIEV (SEQ ID NO: 24).

The Tn3 scaffold may have the amino acid sequence as shown in SEQ ID NO: 1 and described above or it may have one or more amino acid residues changes relative to the amino acid sequence as shown in SEQ ID NO: 1. For example, if the scaffold has amino acid sequence changes relative to those shown in SEQ ID NO: 1, the changes may be to one of the linkers. The Tn3 scaffold may comprise a Gly15 linker separating two CD40L-specific monomers and a Gly10 linker separating a CD40L-specific monomer from an HSA sequence. Both or one of these linkers may be altered, and may be replaced with an amino acid sequence of (GₘX)ₙ wherein X is Serine (S), Alanine (A), Glycine (G), Leu (L), Isoleucine (I), or Valine (V); m and n are integer values; m is 1, 2, 3 or 4; and, n is 1, 2, 3, 4, 5, 6, or 7. For example, one or both linkers may be altered to have an amino acid sequence that comprises one of GGGGSGGGGS (SEQ ID NO: 7), GGGGSGGGGSGGGGS (SEQ ID NO: 8), GGGGGGGGGG (SEQ ID NO: 9) or GGGGGGGGGGGGGGG (SEQ ID NO: 10). If the Tn3 scaffold has an amino acid sequence relative to the amino acid sequence as provided in SEQ ID NO: 1, it may be due to a changes or changes in the HSA amino acid sequence fused to the two CD40L-specific monomers. The HSA fused to the two CD40L-specific monomers may be altered to relative to the HSA fused to the two CD40L-specific Tn3 monomers, except for at least one amino acid substitution, numbered relative to the position in full length mature HSA, at a position selected from the group consisting of 407, 415, 463, 500, 506, 508, 509, 511, 512, 515, 516, 521, 523, 524, 526, 535, 550, 557, 573, 574, and 580; wherein the at least one amino acid substitution does not comprise a lysine (K) to glutamic acid (E) at position 573.

Exemplary sequences for a Tn3 scaffolds are shown in Table 1. In aspects, a Tn3 scaffold comprises at least about or at most about 50%, 55%, 60%, 65%, 70%, 75%, 80%, 85%, 90%, 95%, 96%, 97%, 98%, 99%, or up to about 100% identity with any one of SEQ ID NO: 1-SEQ ID NO: 25 shown in Table 1. In aspects, any one of the sequences from Table 1 can be modified. In aspects, a modification comprises one or more truncations, deletions, insertions, and combinations thereof. A modification can occur at any of the residues provided in Table 1 and in any number of residues from Table 1. In aspects, a modification can comprise from 1-3, 1-5, 1-10, 5-20, 1-3, 1-5, 1-10, 1-20, 3-8, 3-10, 3-15, 5-8, 5-10, or 5-20 residues. In aspects, a modification can occur in up to 1, 2, 3, 4, 5, 6, 7, 8, 9, 10, 11, 12, 13, 14, 15, 20, 30, 40, 50, 60, 70, 80, 90, 100, 200, 300, 400, or 450 residues.

TABLE 1
Exemplary sequences for a Tn3 scaffold
comprising a CD40L-specific monomer subunit

| SEQ | | |
|---|---|---|
| ID | | |
| NO | ID | Sequence |
| 1 | VIB4920 | SQIEVKDVTDTTALITWSDDFGEYVWCELTYGIKDVPGDRT |
| | (342-G15-342- | TIDLWYHHAHYSIGNLKPDTEYEVSLICRSGDMSSNPAKETF |
| | G10-HSAC34S | TTGGGGGGGGGGGGGGGRLDAPSQIEVKDVTDTTALITWS |
| | Bivalent | DDFGEYVWCELTYGIKDVPGDRTTIDLWYHHAHYSIGNLK |
| | construct 2- | PDTEYEVSLICRSGDMSSNPAKETFTTGGGGGGGGGGDAH |
| | All GLY | KSEVAHRFKDLGEENFKALVLIAFAQYLQQSPFEDHVKLVN |
| | linkers HSA | EVTEFAKTCVADESAENCDKSLHTLFGDKLCTVATLRETYG |
| | underlined) | EMADCCAKQEPERNECFLQHKDDNPNLPRLVRPEVDVMCT |
| | | AFHDNEETFLKKYLYEIARRHPYFYAPELLFFAKRYKAAFT |
| | | ECCQAADKAACLLPKLDELRDEGKASSAKQRLKCASLQKF |
| | | GERAFKAWAVARLSQRFPKAEFAEVSKLVTDLTKVHTECC |
| | | HGDLLECADDRADLAKYICENQDSISSKLKECCEKPLLEKS |
| | | HCIAEVENDEMPADLPSLAADFVESKDVCKNYAEAKDVFL |
| | | GMFLYEYARRHPDYSVVLLLRLAKTYETTLEKCCAAADPH |
| | | ECYAKVFDEFKPLVEEPQNLIKQNCELFEQLGEYKFQNALL |
| | | VRYTKKVPQVSTPTLVEVSRNLGKVGSKCCKHPEAKRMPC |
| | | AEDYLSVVLNQLCVLHEKTPVSDRVTKCCTESLVNRRPCFS |
| | | ALEVDETYVPKEFNAETFTFHADICTLSEKERQIKKQTALVE |
| | | LVKHKPKATKEQLKAVMDDFAAFVEKCCKADDKETCFAE |
| | | EGKKLVAASQAALGL |
| 2 | CD40L- | IEVKDVTDTTALITWSDDFGEYVWCELTYGIKDVPGDRTTI |
| | specific Tn3 | DLWYHHAHYSIGNLKPDTEYEVSLICRRGDMSSNPAKETFT |
| | monomer with | T |
| | affinity mature | |
| | variant | |
| | Clone 342- | |
| | Affinity | |
| | mature variant | |
| | (W/WT FG | |
| | loop; W/O N- | |
| | Term A, C- | |
| | Term Linker | |
| | and His8Tag | |
| 3 | CD40L- | IEVKDVTDTTALITWSDDFGEYVWCELTYGIKDVPGDRTTI |
| | specific Tn3 | DLWYHHAHYSIGNLKPDTEYEVSLICRSGDMSSNPAKETFT |
| | monomer with | T |
| | affinity mature | |
| | variant | |
| | Clone 342- | |
| | Affinity | |
| | mature variant | |
| | (W/FG loop | |
| | variant RR→ | |
| | RS underlined) | |
| 4 | Human serum | DAHKSEVAHRFKDLGEENFKALVLIAFAQYLQQSPFEDHVK |
| | albumin | LVNEVTEFAKTCVADESAENCDKSLHTLFGDKLCTVATLRE |
| | variant | TYGEMADCCAKQEPERNECFLQHKDDNPNLPRLVRPEVDV |
| | | MCTAFHDNEETFLKKYLYEIARRHPYFYAPELLFFAKRYKA |
| | | AFTECCQAADKAACLLPKLDELRDEGKASSAKQRLKCASL |
| | | QKFGERAFKAWAVARLSQRFPKAEFAEVSKLVTDLTKVHT |
| | | ECCHGDLLECADDRADLAKYICENQDSISSKLKECCEKPLLE |
| | | KSHCIAEVENDEMPADLPSLAADFVESKDVCKNYAEAKDV |
| | | FLGMFLYEYARRHPDYSVVLLLRLAKTYETTLEKCCAAAD |
| | | PHECYAKVFDEFKPLVEEPQNLIKQNCELFEQLGEYKFQNA |
| | | LLVRYTKKVPQVSTPTLVEVSRNLGKVGSKCCKHPEAKRM |
| | | PCAEDYLSVVLNQLCVLHEKTPVSDRVTKCCTESLVNRRPC |
| | | FSALEVDETYVPKEFNAETFTFHADICTLSEKERQIKKQTAL |
| | | VELVKHKPKATKEQLKAVMDDFAAFVEKCCKADDKETCF |
| | | AEEGKKLVAASQAALGL |
| 5 | beta strand “A” | IEV |
| | within a | |
| | CD40L- | |
| | specific | |
| | monomer | |
| 6 | beta strand “B” | ALITW |
| | within a | |
| | CD40L- | |
| | specific | |
| | monomer | |
| 7 | Linker | GGGGSGGGGS |
| 8 | Linker | GGGGSGGGGSGGGGS |
| 9 | Linker | GGGGGGGGGG |
| 10 | Linker | GGGGGGGGGGGGGGG |
| 11 | AB loop | KDVTDTT |
| 12 | BC loop | SDDFGEYVW |
| 13 | CD loop | KDVPGDR |
| 14 | DE loop | WYHHAH |
| 15 | EF loop | GNLKPDTE |
| 16 | FG loop | RSGDMSSNPA |
| 17 | beta strand “C” | CELTYGI |
| | within a | |
| | CD40L- | |
| | specific | |
| | monomer | |
| 18 | beta strand “D” | TTIDL |
| | within a | |
| | CD40L- | |
| | specific | |
| | monomer | |
| 19 | beta strand “E” | YSI |
| | within a | |
| | CD40L- | |
| | specific | |
| | monomer | |
| 20 | beta strand “F” | YEVSLIC |
| | within a | |
| | CD40L- | |
| | specific | |
| | monomer | |
| 21 | beta strand “G” | KETFTT |
| | within a | |
| | CD40L- | |
| | specific | |
| | monomer | |
| 22 | CD40L- | SQIEVKDVTDTTALITWSDDFGEYVWCELTYGIKDVPGDRT |
| | specific Tn3 | TIDLWYHHAHYSIGNLKPDTEYEVSLICRSGDMSSNPAKETF |
| | monomer | TT |
| | Clone 342- | |
| | Affinity | |
| | mature variant | |
| | (W/FG loop | |
| | variant RR→ | |
| | RS underlined) | |
| 23 | beta strand | RLDAPSQIEV |
| | sequence “A” | |
| | within a | |
| | CD40L- | |
| | specific | |
| | monomer | |
| 24 | beta strand “A” | SQIEV |
| | within a | |
| | CD40L- | |
| | specific | |
| | monomer | |
| 25 | CD40L- | RLDAPSQIEVKDVTDTTALITWSDDFGEYVWCELTYGIKDV |
| | specific Tn3 | PGDRTTIDLWYHHAHYSIGNLKPDTEYEVSLICRSGDMSSNP |
| | monomer | AKETFTT |

If the Tn3 scaffold has amino acid sequence changes relative to those shown in SEQ ID NO: 1, the changes may be to the amino acid sequence of one or both of the CD40L-specific Tn3 monomers, so long as it does not adversely effect in vivo efficacy of the scaffold, e.g., change in amino acid sequence such that one or both CD40L-specific Tn3 monomers have the amino acid sequence as shown in SEQ ID NO: 2, SEQ ID NO: 3, SEQ ID NO: 22, and SEQ ID NO: 25. In aspects, the first one or two N-terminal amino acid residues (SQ) may be absent and/or substituted with alternative amino acid residues. In aspects, a Tn3 scaffold comprises a monomer subunit comprising SEQ ID NO: 22, SEQ ID NO: 25, or both SEQ ID NO: 22 and SEQ ID NO: 25.

In aspects, a Tn3 scaffold comprises at least one CD40L-specific monomer subunit bound to a heterologous moiety. In aspects, this heterologous moiety is selected from the group consisting of: a protein, a peptide, a protein domain, a linker, a drug, a toxin, a cytotoxic agent, an imaging agent, a radionuclide, a radioactive compound, an organic polymer, an inorganic polymer, a polyethylene glycol (PEG), biotin, an albumin, a HSA FcRn binding portion, an antibody or fragment thereof, a single chain antibody, a domain antibody, an albumin binding domain, an enzyme, a ligand, a receptor, a binding peptide, a non-FnIII scaffold, an epitope tag, a recombinant polypeptide polymer, a cytokine, and a combination of two or more of said moieties. In aspects, the heterologous moiety is the albumin, and the albumin comprises human serum albumin. In aspects, the heterologous moiety is an antibody. In aspects, the antibody is selected from the group consisting of: an Fc domain of an antibody, an antibody fragment, and a single chain antibody.

In aspects, the heterologous moiety is an antibody. In aspects, the antibody is selected from the group consisting of: an Fc domain of an antibody, an antibody fragment, and a single chain antibody.

In aspects, the heterologous moiety is an imaging agent; for example, a radionuclide or biotin. In aspects, the heterologous moiety is a drug; for example, a cytotoxic agent or a radioactive compound.

In aspects, the heterologous moiety comprises PEG. In aspects, the Tn3 scaffold comprises at least one CD40L-specific monomer subunit fused or conjugated directly or via a linker to PEG. In aspects, both CD40L-specific monomer subunits are fused, conjugated, or connected via a linker to PEG. In aspects, the Tn3 scaffold comprises at least one (e.g., two) CD40L-specific monomer subunit fused or conjugated directly or via a linker to PEG.

In aspects, the heterologous moiety comprises albumin. In aspects, the Tn3 scaffold comprises at least one CD40L-specific monomer subunit fused or conjugated directly or via a linker to an albumin. In aspects, this albumin is HSA. In aspects, this HSA is a variant HSA. In aspects, the amino acid sequence of the variant HSA is SEQ ID NO: 4. In aspects, the variant HSA has at least one improved property compared with a native HSA or a native HSA fragment. In aspects, the amino acid sequence of the variant HSA is SEQ ID NO: 4 or a sequence having at least about 80%, 85%, 90%, 95%, 96%, 97%, 98%, 99%, or up to 100% identity to SEQ ID NO: 4. In aspects, the improved property is an altered plasma half-life compared with the plasma half-life of a native HSA or a native HSA fragment. In aspects, the altered plasma half-life is a longer plasma half-life compared with the plasma half-life of a native HSA or a native HSA fragment. In aspects, the altered plasma half-life is a shorter plasma half-life compared with the plasma half-life of a native HSA or a native HSA fragment.

### Dosing

In aspects, any of the compositions comprising a Tn3 scaffold of the disclosure can be administered in any form. In aspects, a Tn3 scaffold is administered intravenously, subcutaneously, orally, intramuscularly, intrathecally, sublingually, rectally, vaginally, cutaneously, systemically, topically, transdermally, or by way of inhalation. In aspects, the Tn3 scaffold is administered intravenously. In aspects, the Tn3 scaffold is administered by intravenous infusion.

A Tn3 scaffold of the disclosure can be administered at any dose. In aspects, a Tn3 scaffold is administered at a dose from about: 800 mg, 850 mg, 900 mg, 950 mg, 1000 mg, 1050 mg, 1100 mg, 1150 mg, 1200 mg, 1250 mg, 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, 4500 mg, 4550 mg, 4600 mg, 4650 mg, 4700 mg, 4750 mg, 4800 mg, 4850 mg, 4900 mg, 4950 mg, or about 5000 mg. Any of the aforementioned dosages may be effective dosages for a method comprising treatment, reduction, or elimination.

In aspects, a Tn3 scaffold is administered at a dose of between about: 800-5000 mg, 900-4900 mg, 1000-4800 mg, 1100-4700 mg, 1200-4600 mg, or 1300-4500 mg. In aspects, a Tn3 scaffold is administered at a dose selected from the group consisting of: 1300 mg, 1350 mg, 1400 mg, 1450 mg, 1500 mg, 1550 mg, 1600 mg, 1650 mg, 1700 mg, 1750 mg, 1800 mg, 1850 mg, 1900 mg, 1950 mg, 2000 mg, 2050 mg, 2100 mg, 2150 mg, 2200 mg, 2250 mg, 2300 mg, 2350 mg, 2400 mg, 2450 mg, 2500 mg, 2550 mg, 2600 mg, 2650 mg, 2700 mg, 2750 mg, 2800 mg, 2850 mg, 2900 mg, 2950 mg, 3000 mg, 3050 mg, 3100 mg, 3150 mg, 3200 mg, 3250 mg, 3300 mg, 3350 mg, 3400 mg, 3450 mg, 3500 mg, 3550 mg, 3600 mg, 3650 mg, 3700 mg, 3750 mg, 3800 mg, 3850 mg, 3900 mg, 3950 mg, 4000 mg, 4050 mg, 4100 mg, 4150 mg, 4200 mg, 4250 mg, 4300 mg, 4350 mg, 4400 mg, 4450 mg, and 4500 mg. In aspects, a Tn3 scaffold is administered at a dose selected from the group consisting of: 1500 mg and 3000 mg. In aspects, a Tn3 scaffold is administered at a dose of about 1500 mg. In aspects, a Tn3 scaffold is administered at a dose of about 3000 mg.

### Dosing Frequency

In aspects, a Tn3 scaffold of the disclosure is administered on a schedule that provides optimal results. In aspects, a Tn3 scaffold is administered to a subject in need thereof about once a week, about twice a week, about every two weeks, about once a month, about every four weeks, about every two months, about every 3 months, about every 12 weeks, about every fifteen weeks, about every sixteen weeks, about every four months, about every five months, about every six months, or semiannually. Any number of administrations may be provided to a subject in need thereof.

A Tn3 scaffold of the disclosure can be administered from about 1-10, 10-50, 50-75, 75-100, 100-200, 200-300 total doses, or up to the lifetime of a subject. In aspects, a Tn3 scaffold is administered about 1, 2, 3, 4, 5, 6, 7, 8, 9, or up to about 10 doses. In aspects, a Tn3 scaffold is administered at least about or at most about 2, 3, 4, or 5 total doses.

In aspects, a subject is administered an effective dose on about every Day 1, Day 2, Day 3, Day 4, Day 5, Day 6, 1 week, 2 weeks, 3 weeks, 4 weeks, 5 weeks, 6 weeks, 7 weeks, 8 weeks, 9 weeks, 10 weeks, 11 weeks, 12 weeks, 3 months, 4 months, 5 months, 6 months, 7 months, 8 months, 9 months, 10 months, 11 months, 1 year, 2 years, 3 years, 4 years, or 5 years, or up to the lifetime of a subject, post treatment initiation. In aspects, a subject receives an effective dose on Day 1, Day 15, Day 29, and Day 57 post treatment initiation. In aspects, a subject receives 1500 mg-3000 mg of a Tn3 scaffold on Day 1, Day 15, Day 29, and Day 57 post treatment initiation. In aspects, a subject is administered from about 1500 mg-3000 mg of a Tn3 scaffold every 2 weeks for about 3 administrations then every 4 weeks thereafter. In aspects, a subject is administered about 1500 mg of a Tn3 scaffold every 2 weeks for about 3 administrations then every 4 weeks thereafter. In aspects, a subject is administered about 3000 mg of a Tn3 scaffold at week 1, 4, and 12 then every 12 weeks thereafter. In aspects, a subject in need thereof is administered 1500 mg of a Tn3 scaffold on Day 1, Day 15, Day 29, and Day 57 post treatment initiation. In aspects, a subject in need thereof is administered 1500 mg of a Tn3 scaffold on day 1 and day 57 post treatment initiation. In aspects, a subject in need thereof is administered 3000 mg of a Tn3 scaffold on Day 1, Day 15, Day 29, and Day 57 post treatment initiation. In aspects, a subject in need thereof is administered 3000 mg of the Tn3 scaffold on Day 1 and Day 57 post treatment initiation, and then every 6 months thereafter as needed. In aspects, a subject is administered an initial dose of a Tn3 scaffold of the disclosure of about 1500-3000 mg every 2 weeks for at least 2, at least 3, or more administrations, and then every 4 weeks thereafter. In aspects, a subject is administered an initial dose of a Tn3 scaffold of the disclosure of about 1500 mg every 2 weeks for at least 2, at least 3, or more administrations, and then every 4 weeks thereafter. In aspects, a subject is administered an initial dose of a Tn3 scaffold of the disclosure of about 3000 mg every 2 weeks for at least 2, at least 3, or more administrations, and then every 12 weeks thereafter. In aspects, the subject is administered 1500 mg of a Tn3 scaffold of the disclosure every 4 weeks, with a loading dose of 1500 mg of the Tn3 scaffold at week 2. In aspects, the subject is administered 3000 mg of a Tn3 scaffold of the disclosure every 12 weeks, with a loading dose of 3000 mg of the Tn3 scaffold at week 4. In aspects, the subject is administered 1500 mg of a Tn3 scaffold of the disclosure every 4 weeks, with a loading dose of 1500 mg of the Tn3 scaffold at week 2. In aspects, the subject is administered 3000 mg of a Tn3 scaffold comprising SEQ ID NO: 1 every 12 weeks, with a loading dose of 3000 mg of the Tn3 scaffold at week 4. In aspects, the subject is administered 3000 mg of a Tn3 scaffold comprising SEQ ID NO: 22 and/or SEQ ID NO: 25 every 12 weeks, with a loading dose of 3000 mg of the Tn3 scaffold at week 4. In aspects, the subject is administered 3000 mg of a Tn3 scaffold comprising SEQ ID NO: 1 every 12 weeks, with a loading dose of 3000 mg of the Tn3 scaffold at week 4. In aspects, the subject is administered 1500 mg of a Tn3 scaffold comprising SEQ ID NO: 22 or SEQ ID NO: 25 every 4 weeks, with a loading dose of 1500 mg of the Tn3 scaffold at week 2. In aspects, the subject is administered 3000 mg of a Tn3 scaffold comprising SEQ ID NO: 22 or SEQ ID NO: 25 every 12 weeks, with a loading dose of 3000 mg of the Tn3 scaffold at week 4.

Methods of treatment or prevention of the disclosure comprise administering a Tn3 scaffold of the disclosure to a subject in need thereof. In aspects, a method of treatment comprises administering an effective amount of a Tn3 scaffold to a subject in need every 2 weeks for about 3 administrations (±2 administrations), followed by an administration of a Tn3 scaffold every 4 weeks thereafter. In aspects, a method of treatment comprises administering an effective amount of a Tn3 scaffold to a subject in need on week 1, 4, and 12, followed by an administration of a Tn3 scaffold every 12 weeks thereafter. In aspects, a method of treatment comprises administering from about 1400 mg-1600 mg of a Tn3 scaffold to a subject in need every 2 weeks for about 3 administrations (±2 administrations), followed by an administration of a Tn3 scaffold every 4 weeks thereafter. In aspects, a method of treatment comprises administering from about 2000 mg-4000 mg of a Tn3 scaffold to a subject in need on week 1, 4, and 12, followed by an administration of a Tn3 scaffold every 12 weeks thereafter. In aspects, a method of treatment comprises administering from about 1500 mg of a Tn3 scaffold to a subject in need every 2 weeks for about 3 administrations (±2 administrations), followed by an administration of the Tn3 scaffold every 4 weeks thereafter. In aspects, a method of treatment comprises administering from about 3000 mg of a Tn3 scaffold to a subject in need on week 1, 4, and 12, followed by an administration of the Tn3 scaffold every 12 weeks thereafter. In aspects, administrations continue every 4 weeks thereafter up to about 1 year, 2 years, 3 years, 4 years, or 5 years. In aspects, administrations of a Tn3 scaffold continue until a subject's death. Any of the aforementioned administrations can deviate by about 0-5 days, 0-4 days, 0-3 days, 0-2 days, or by about 1 day.

In aspects, a subject receives an effective dose of a Tn3 scaffold on Day 1, Day 15 (1 day), Day 29 (±3 days), and Day 57 (±3 days) post treatment initiation. In aspects, a subject in need thereof is administered 1500 mg of a Tn3 scaffold on Day 1, Day 15 (±1 day), Day 29 (±3 days), and Day 57 (±3 days) post treatment initiation, and then every 6 months thereafter as needed. In aspects, a subject in need thereof is administered 1500 mg of a Tn3 scaffold on Day 1, Day 15 (±1 day), and Day 29 (±3 days) post treatment initiation. In aspects, a subject in need thereof is administered 1500 mg of a Tn3 scaffold on Day 1 and Day 57 (±3 days) post treatment initiation. In aspects, a subject in need thereof is administered 3000 mg of a Tn3 scaffold on Day 1, Day 15 (±1 day), Day 29 (±3 days), and Day 57 (±3 days) post treatment initiation, and then every 6 months thereafter as needed. In aspects, a subject in need thereof is administered 3000 mg of a Tn3 scaffold on Day 1, Day 15 (±1 day), and Day 29 (±3 days) post treatment initiation. In aspects, a subject in need thereof is administered 3000 mg of a Tn3 scaffold on Day 1 and Day 57 (±3 days) post treatment initiation, and then every 6 months thereafter as needed.

In aspects, a subject in need thereof receives a dose of a Tn3 scaffold on Day 1, Day 15 (±1 days), Day 29 (±3 days), Day 57 (±3 days), Day 85 (±3 days), Day 113 (±3 days), and Day 141 (±3 days). In aspects, a subject in need thereof receives an effective dose of a Tn3 scaffold on Day 169 (±3 days), Day 197 (±3 days), Day 225 (±3 days), Day 253 (±3 days), Day 281 (±3 days).

In aspects, a subject in need thereof is administered an effective dose of a Tn3 scaffold once every 2-4 weeks. In aspects, a subject in need thereof is administered an effective dose of a Tn3 scaffold once every 2 weeks, 4 weeks, or 12 weeks. In aspects, a subject in need thereof is administered 1500 mg of a Tn3 scaffold once every 2 weeks for at least 3 doses, once every 4 weeks for at least 4 doses, once every 4 weeks for at least 5 doses, or a combination thereof. In aspects, a subject in need thereof is administered 3000 mg of a Tn3 scaffold once every 2 weeks for at least 3 doses, once every 4 weeks for at least 4 doses, once every 4 weeks for at least 5 doses, or a combination thereof. In aspects, a subject in need thereof is administered an effective dose of a Tn3 scaffold as an induction dose and as a maintenance dose thereafter. In aspects, 3000 mg of a Tn3 scaffold is administered every 3 months. In aspects, 3000 mg of a Tn3 scaffold is administered every 12 weeks.

In aspects, a Tn3 scaffold is administered at a dose of about 1500 mg once about every 2 weeks for at least 2 doses and is administered about once a month thereafter. In aspects, a Tn3 scaffold is administered at a dose of about 1500 mg once about every 2 weeks for at least 3 doses and is administered about once a month thereafter. In aspects, a Tn3 scaffold is administered at a dose of about 1500 mg once about every 2 weeks for at least 3 doses and is administered every 4 weeks thereafter. In aspects, a Tn3 scaffold is administered at a dose of about 1500 once about every month, once about every two months, or once about every three months. In aspects, a Tn3 scaffold is administered at a dose of about 3000 mg once about every month, once about every two months, or once about every three months. In aspects, a Tn3 scaffold is administered for two or more doses. In aspects, the subject is administered 1500 mg of a Tn3 scaffold of the disclosure every 4 weeks, with a loading dose of 1500 mg of the Tn3 scaffold at week 2. In aspects, the subject is administered 3000 mg of a Tn3 scaffold of the disclosure every 12 weeks, with a loading dose of 3000 mg of the Tn3 scaffold at week 4. In aspects, the subject is administered 1500 mg of a Tn3 scaffold comprising SEQ ID NO: 1 every 4 weeks, with a loading dose of 1500 mg of the Tn3 scaffold at week 2. In aspects, the subject is administered 3000 mg of a Tn3 scaffold comprising SEQ ID NO: 1 every 12 weeks, with a loading dose of 3000 mg of the Tn3 scaffold at week 4. In aspects, the subject is administered 1500 mg of a Tn3 scaffold comprising SEQ ID NO: 22 or SEQ ID NO: 25 every 4 weeks, with a loading dose of 1500 mg of the Tn3 scaffold at week 2. In aspects, the subject is administered 3000 mg of a Tn3 scaffold comprising SEQ ID NO: 22 or SEQ ID NO: 25 every 12 weeks, with a loading dose of 3000 mg of the Tn3 scaffold at week 4.

In aspects, the subject is administered 1500 mg of a Tn3 scaffold of the disclosure every 4 weeks, with a loading dose of 1500 mg of the Tn3 scaffold at week 2. In aspects, the subject is administered 3000 mg of a Tn3 scaffold comprising SEQ ID NO: 1 every 12 weeks, with a loading dose of 3000 mg of the Tn3 scaffold at week 4. In aspects, the subject is administered 3000 mg of a Tn3 scaffold comprising SEQ ID NO: 22 and/or SEQ ID NO: 25 every 12 weeks, with a loading dose of 3000 mg of the Tn3 scaffold at week 4. In aspects, the subject is administered 3000 mg of a Tn3 scaffold comprising SEQ ID NO: 1 every 12 weeks, with a loading dose of 3000 mg of the Tn3 scaffold at week 4. In aspects, the subject is administered 1500 mg of a Tn3 scaffold comprising SEQ ID NO: 22 or SEQ ID NO: 25 every 4 weeks, with a loading dose of 1500 mg of the Tn3 scaffold at week 2. In aspects, the subject is administered 3000 mg of a Tn3 scaffold comprising SEQ ID NO: 22 or SEQ ID NO: 25 every 12 weeks, with a loading dose of 3000 mg of the Tn3 scaffold at week 4.

### Methods of Treatment

Provided are methods of treating autoimmune disease. Any autoimmune disease may be treated utilizing compositions of the disclosure. In several autoimmune diseases, the CD40/CD40L pathway is activated on a variety of cell types, including T cells, B Cells, antigen-presenting cells, and activated epithelial cells. In aspects, compositions of the disclosure can be utilized in an autoimmune disease where the CD40/CD40L pathway is activated on a variety of cell types, including T cells, B Cells, antigen-presenting cells, and activated epithelial cells. In aspects, a Tn3 scaffold of the disclosure blocks costimulatory signals between T cells and CD40-expressing B cells, disrupting the development of germinal centers, pathogenic B cells, plasma cells, and autoantibodies that are hallmarks of autoimmune disease.

In aspects, an autoimmune disease is systemic lupus erythematosus (SLE), lupus nephritis, cutaneous lupus erythematosus (CLE), Sjögren's syndrome, dermatomyositis, myositis, sclerosis, diabetes, Hashimoto's disease, autoimmune adrenal insufficiency, anemia, rheumatic carditis, psoriasis, arthritis (e.g., rheumatoid arthritis; RA), inflammation, chronic rheumatism, vitiligo, alopecia areata, hidradenitis suppurativa, celiac disease, graft versus host disease (GVHD), myocardial infarction, a Type-1 interferonopathy, and any combination thereof. In aspects, an autoimmune disease or other disease or condition comprises increased levels of a T or B cell biomarker of the disclosure.

### Rheumatoid Arthritis

In aspects herein, methods are directed to treat RA. In aspects, a method comprises administering a Tn3 scaffold of the disclosure. In aspects, a Tn3 scaffold is administered to a subject in need thereof to treat RA using any of the dosing schedules disclosed herein. In aspects, a Tn3 scaffold is administered at a dose of about 1500 mg once about every 2 weeks for at least 2 doses and is administered about once a month thereafter. In aspects, a Tn3 scaffold is administered at a dose of about 1500 mg once about every 2 weeks for at least 3 doses and is administered about once a month thereafter. In aspects, a Tn3 scaffold is administered at a dose of about 1500 once about every month, once about every two months, or once about every three months. In aspects, a Tn3 scaffold is administered at a dose of about 3000 mg once about every month, once about every two months, or once about every three months. In aspects, a Tn3 scaffold is administered for two or more doses.

In aspects of the methods disclosed herein, a subject in need thereof has been administered one or more standard of care therapies for the treatment of RA prior to the administration of a Tn3 scaffold. In aspects, provided methods are described in Int'l Appl. No. PCT/US2022/077192, which is incorporated herein by reference in its entirety.

### Sjögren's Syndrome

In aspects herein, methods are directed to treat SS. In aspects, a method comprises administering a Tn3 scaffold of the disclosure. In aspects, a Tn3 scaffold is administered to a subject in need thereof to treat SS using any of the dosing schedules disclosed herein. In aspects, a Tn3 scaffold is administered at a dose of about 1500 mg once about every 2 weeks for at least 2 doses and is administered about once a month thereafter. In aspects, a Tn3 scaffold is administered at a dose of about 1500 mg once about every 2 weeks for at least 3 doses and is administered about once a month thereafter. In aspects, a Tn3 scaffold is administered at a dose of about 1500 once about every month, once about every two months, or once about every three months. In aspects, a Tn3 scaffold is administered at a dose of about 3000 mg once about every month, once about every two months, or once about every three months. In aspects, a Tn3 scaffold is administered for two or more doses.

In aspects, a method comprises treating a patient with SS. In aspects, a method comprises treating a patient with SS with a European League Against Rheumatism (EULAR) Sjögren's Syndrome Disease Activity Index (ESSDAI) of about 1, about 2, about 3, about 5, about 5, about 6, about 7, about 8, about 9, about 10, about 15, about 20, about 25, about 30, about 40, about 50, about 60, about 70, about 80, about 90, about 100 or greater. In aspects, a method comprises treating a patient with SS with a EULAR Sjögren's Syndrome Patient Reported Index (ESSPRI) score of about 1, about 2, about 4, about 5, about 6, about 7, about 8, about 9, about 10, about 15, about 20 or greater.

In aspects, a method comprises treating a patient in need thereof. In aspects, a method comprises treating a patient with SS. In aspects, a method comprises treating a patient with SS with moderate systemic disease activity. In aspects, a method comprises treating a patient with SS with high systemic disease activity. In aspects, a method comprises treating a patient with SS with moderate to high systemic disease activity. In aspects, a method comprises treating a patient with SS with moderate to high systemic disease activity by administering a Tn3 scaffold in any dose at in any schedule disclosed herein. In aspects, moderate to high systemic disease activity may be defined by ESSDAI≥5. In aspects, a method comprises treating a patient with SS with moderate to high (or severe) subjective symptoms by administering a Tn3 scaffold. In aspects, moderate to high (or severe) subjective symptoms may be defined by EULAR Sjögren's Syndrome Patient Reported Index (ESSPRI) score≥5 and residual stimulated salivary flow but with mild systemic disease activity defined by ESSDAI score<5. In aspects, a Tn3 scaffold is administered at a dose of 1500 mg. In aspects, a Tn3 scaffold is administered at a dose of 3000 mg.

Provided are also diagnostic methods helpful in identifying subjects that may benefit from administration of a composition of the disclosure. In aspects, a method comprises determining if a sample of a subject comprises a biomarker of the disclosure to inform treatment. For example, a method can comprise determining if a subject in need is positive for one or more cells selected from the group consisting of Ki67+CD27+ memory cells, CD27 high CD38 high plasmablasts, and CD11c high atypical memory cells, and administering a composition of the disclosure if a positive result is obtained. In aspects, a method can comprise determining if a subject in need is positive for one or more markers or cells selected from the group consisting of CXCL13, RF antibodies, Ki67+CD27+ memory cells, CD27 high CD38 high plasmablasts, and CD11c high atypical memory cells, and administering a composition of the disclosure if a positive result is obtained. In aspects, a positive result comprises an increased number of the one or more cells as compared to a healthy baseline level of the subject. In aspects, a positive result comprises an increased number of the one or more cells as compared to an otherwise comparable healthy subject. In aspects, a method comprises routinely determining if a subject in need is positive for the one or more cells and administering treatment as needed to revert a level of the one or more cells to a baseline value of the subject. A baseline level can refer to a level of a biomarker before administration of a composition of the disclosure and therefore can be associated with disease. In aspects, a baseline level can refer to a level of a biomarker after administration of a composition of the disclosure whereby the subject comprises controlled disease or no disease (e.g., healthy baseline).

### Assessment

In aspects, a subject is assessed. An assessment can occur at any point before, during, or after administration with a Tn3 scaffold. An assessment can occur at any point before, during, or after administration with a Tn3 scaffold. In aspects, an assessment is performed before administration. In aspects, an assessment is performed during administration. In aspects, an assessment is performed post administration.

Any of the below-referenced assessments can occur at any time. In aspects, a subject is assessed by the minute, hourly, daily, weekly, monthly, or yearly. In aspects, an assessment is completed twice daily, biweekly, bimonthly, or semiannually. In aspects, an assessment is performed from day −10, −9, −8, −7, −6, −5, −4, −3, −2, −1, 0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, 141, 145, 150,155, 160, 165, 169, 170, 180, 190, 197, 200, 205, 210, 215, 220, 225, 230, 235, 240, 250, 252, 253, 255, 260, 265, 270, 280, 281, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, or up to about day 365±7 days post-treatment.

As discussed above, in aspects a subject undergoes routine evaluation to determine a level of cells expressing a biomarker of the disclosure (or a level of a biomarker). In aspects, a level of cells expressing a biomarker of the disclosure correlates with disease activity. For example, T cells and/or B cells may be costimulated in a diseased state and cause upregulation of cells expressing biomarkers of the disclosure, thereby leading to increased disease in a subject. Routine evaluation of a subject may allow for monitoring and appropriate intervention to control or reduce disease. Routine evaluation may comprise daily, weekly, monthly, or yearly monitoring. In aspects, a subject is evaluated from 1, 2, 3, 4, 5, 6, 7, 8, 9, 10, 11, or 12 times a year. In aspects, a subject is evaluated from 1-3, 2-4, 3-5, 4-6, or 5-7 times a year. In aspects, a subject is evaluated every year, 2 years, 3 years, 4 years, or 5 years.

In aspects, a treatment of SS may be characterized by a reduction of at least about 5%, about 10%, about 15%, about 20%, about 30%, about 40%, about 50%, about 60%, about 70%, about 80%, about 90%, about 95%, about 96%, about 97%, about 98%, about 99%, or up to about 100% of clinical symptoms of the disease or disorder, or by a reduction in inflammation, or by a reduction in biomarkers of the disease or disorder, relative to their levels prior to the treatment with a Tn3 scaffold. A reduction of any of these symptoms, or inflammation, or biomarkers, may be a reduction in the symptoms, or inflammation or biomarkers of at least about 10%, 15%, 20%, 25%, about 30%, about 40%, about 50%, about 60%, about 70%, about 75%, about 80%, about 85%, about 90%, about 95%, about 96%, about 97%, about 98%, about 99%, or up to about 100% relative to their levels prior to the initiation of treatment with a Tn3 scaffold. A reduction may be such that the SS is characterized as being in remission.

In aspects, administration of a compositions of the disclosure is effective in reducing a level of a biomarker, a particular cell type, or a level of cells expressing a biomarker described herein. In aspects, a biomarker is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject. In aspects, a level of B cells is reduced by at least about 1-fold, 5-fold, 10-fold, 30-fold, 50-fold, 75-fold, 100-fold, 150-fold, or 200-fold following administration of a composition of the disclosure.

In aspects, the level of Ki67+CD27+ memory cells is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject. In aspects, the level of CD27 high CD38 high plasmablasts is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject. In aspects, the level of CD11c high atypical typical memory cells is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject. In aspects, the level of plasmablasts is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject. In aspects, the level of CXCL13 is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject. In aspects, the level of RF antibodies is reduced by at least or by at most about 5%, about 10%, about 15%, about 20%, about 30%, about 40%, about 50%, about 60%, about 70%, about 80%, about 90%, about 95%, about 96%, about 97%, about 98%, about 99%, or up to about 100% relative to a baseline level of the subject or a level in an untreated otherwise comparable subject.

In aspects, efficacy of treatment may be determined using the European League Against Rheumatism (EULAR) Sjögren's Syndrome Disease Activity Index (ESSDAI), EULAR Sjögren's Syndrome Patient Reported Index (ESSPRI), 36-Item Short Form Survey version 2 (SF-36v2), Functional Assessment of Chronic Illness Therapy-Fatigue (FACIT-Fatigue), visual analog scale (VAS) Oral/Ocular, Patient-Reported Outcomes Measurement Information System® (PROMIS-29 Profile v2.1), Ocular Surface Disease Index (OSDI), Patient's Global Impression of Change (PGIC), Patient's Global Impression of Severity (PGIS), Stimulated Salivary Flow Measurement, Schirmer's Test, 28-Joint Assessment, Physician Global Impression of Severity, Clinical EULAR Sjögren's Syndrome Disease Activity Index (ClinESSDAI), and combinations thereof. In aspects, efficacy of treatment may be measured using assessments that are patient-reported outcomes (PRO) instruments.

### Systemic Disease Activity and Patient-Reported Outcomes

### ESSDAI

In aspects, an assessment comprises the ESSDAI. The ESSDAI is a systemic disease activity index that includes organ-by-organ definitions of disease activity (see Seror R, Ravaud P, Bowman S J, Baron G, Tzioufas A, Theander E, et al; EULAR Sjögren's Task Force. EULAR Sjögren's syndrome disease activity index: development of a consensus systemic disease activity index for primary Sjögren's syndrome. Ann Rheum Dis. 2010; 69(6):1103-9, which is incorporated herein by reference). The ESSDAI grades disease activity in 12 domains (cutaneous, respiratory (pulmonary), renal, articular, muscular, peripheral nervous system, central nervous system, hematological, glandular, constitutional, lymphadenopathic, and biological). The weights of each domain were obtained by multiple regression modeling, using the Physician's Global Assessment of Activity as gold standard.

Each domain is weighted from 1 (Biologic domain) to 6 (Muscular domain) and has 3 or 4 levels of activity per domain, ranging from 0 (no activity) to 3 (high activity).

In aspects, the following domains are scored but they may not contribute to the minimum ESSDAI score of 5 required for inclusion in a study of the disclosure: peripheral nervous system, central nervous system, and pulmonary.

The theoretical range of values for the ESSDAI is 0 to 123, with the final score being calculated as follows:

$$Final Score = Sum of all 12 domain scores$$

$$Domain score = Acivity level \times Domain weight$$

$$Low disease activity status is defined as ESSDAI < 5 , moderate disease activity as$$

$$5 \leq ESSDAI \leq 13 and high disease activity as ESSDAI \geq 14.$$

In aspects, a change from baseline in ESSDAI is determined. In aspects, a baseline ESSDAI score is reduced by at least about 1, 2, 3, 4, or 5 points post treatment with a composition provided herein. In aspects, a baseline ESSDAI score is reduced by 3 points post treatment with a composition provided herein. In aspects, a baseline ESSDAI score is reduced by 4 points post treatment with a composition provided herein. In aspects, a post-treatment ESSDAI score is ≤2, ≤5, or ≤13. Administration of a Tn3 scaffold of the disclosure can be effective in reducing an ESSDAI score by at least about or at most about 1, 2, 3, 4, 5, 6, 7, 8, 9, 10, 15, 18, or 20 points as compared to the ESSDAI baseline score prior to the administration. Administration of a Tn3 scaffold of the disclosure can be effective in reducing an ESSDAI score by at least about or at most about 1-5, 3-5, 5-8, 5-10, or 10-15 points as compared to the ESSDAI baseline score prior to the administration. In aspects, administration of a Tn3 scaffold of the disclosure is effective in reducing an ESSDAI score by at least about 6 points. In aspects, administration of a Tn3 scaffold of the disclosure is effective in reducing an ESSDAI score by at least about 6.3 points.

An ESSDAI score can refer to an absolute value or a relative value. For example, a relative value can encompass a difference between a subject treated with a Tn3 scaffold of the disclosure and a subjected treated with a placebo control. In aspects, an ESSDAI score refers to an absolute value, e.g., the reduction of ESSDAI as compared to a baseline level or level determined before treatment. In aspects, an ESSDAI score refers to an relative value. A relative value can refer to a difference between a ESSDAI score between Tn3-scaffold-treated subjects and control-treated subjects. In aspects, a relative value is least squares mean difference between Tn3-scaffold-treated subjects and control-treated subjects.

In aspects, a subject administered a Tn3 scaffold of the disclosure achieves a 3, 4, 5, 6, 7, 8, 9, or 10 point reduction in their ESSDAI as compared to the ESSDAI prior to the administration (e.g., a baseline level). In aspects, a subject administered a Tn3 scaffold of the disclosure achieves a 6.4-point reduction in their ESSDAI as compared to the ESSDAI prior to the administration (e.g., a baseline level).

In aspects, a ESSDAI assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a ESSDAI assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, an ESSDAI assessment can occur at any point before, during, or after administration with a Tn3 scaffold.

### ClinESSDAI

In aspects, an assessment comprises the Clinical EULAR Sjögren's Syndrome Disease Activity Index (ClinESSDAI). ClinESSDAI is a validated SS disease activity index, based on ESSDAI that excludes the biological domain and assigns different weights assigned to each domain. ClinESSDAI was developed in an effort to diminish possible associations between the B-cell biomarkers measured by the ESSDAI biological domain and clinical activity measures. The theoretical range of values for the ClinESSDAI is 0 to 135, and similar to ESSDAI, low-activity status is defined as <5, moderate-activity as 5≤ClinESSDAI≤13 and high-activity as ≥14. ClinESSDAI has been validated and shown to correlate well with ESSDAI and is considered a useful tool to detect change independent of biological effect of the drug. In aspects, a change from baseline in ClinESSDAI is determined. In aspects, a baseline ClinESSDAI score is reduced by at least about 1, 2, 3, 4, or 5 points post treatment with a composition provided herein. In aspects, a post-treatment ClinESSDAI score is ≤2, ≤5, or ≤13.

In aspects, a ClinESSDAI assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a ClinESSDAI assessment to a Tn3 scaffold can be assessed on about Day 85±3, Day 169±3, and Day 309±7 post treatment initiation. In aspects, a ClinESSDAI assessment can occur at any point before, during, or after administration with a Tn3 scaffold.

### ESSPRI

In aspects, an assessment comprises the ESSPRI (see Seror R, Ravaud P, Mariette X, Bootsma H, Theander E, Hansen A, et al. EULAR Sjögren's Task Force. EULAR Sjögren's Syndrome Patient Reported Index (ESSPRI): development of a consensus patient index for primary Sjögren's syndrome. Ann Rheum Dis. 2011; 70(6):968-72, which is incorporated herein by reference). ESSPRI is a self-evaluation tool that was developed in a multicenter international cohort of 230 patients. The ESSPRI uses a 0 to 10 numerical analog scale (ranging from 0 [no symptoms] to 10 [maximal imaginable severity]), one for the assessment of each of the 3 domains: dryness, fatigue, and pain (articular and/or muscular). The weights of the domains are identical, and the mean of the scores of the 3 domains represents the final score. The recall period is stated in each question as “the last 2 weeks.” In aspects, subjects in population #2 achieve an ESSPRI response, defined as ≥1 point or 15% reduction from baseline in ESSPRI score without premature discontinuation of a Tn3 scaffold and without receiving rescue therapy. In aspects, a baseline ESSPRI score is reduced by about 3%, 5%, 8%, 10%, 20%, 30%, 40%, 50%, 60%, 70%, 80%, 90%, 95%, 96%, 97%, 98%, 99%, or up to about 100%. In aspects, a post-treatment ESSPRI score is 0, 1, 2, 3, 4, 5, 6, 7, 8, or 9. In aspects, a post-treatment ESSPRI score is reduced by at least about 1, 2, 3, 4, 5, 6, 7, 8, or 9 points as compared to a pre-treatment ESSPRI score.

In aspects, a ESSPRI assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a ESSPRI assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, an ESSPRI assessment can occur at any point before, during, or after administration with a Tn3 scaffold.

### Functional Assessment of Chronic Illness Therapy (FACIT)-Fatigue

In aspects, an assessment comprises the (FACIT)-Fatigue scale. The (FACIT)-Fatigue Scale is a 13-item subject-completed questionnaire used to assess the impact of fatigue. The (FACIT)-Fatigue Scale recall period is about 7 days. Responses to the (FACIT)-Fatigue Scale range from 0 (Not at all) to 4 (Very much). To calculate the total score, the negatively stated items are reversed by subtracting the response from “4”. Final scores are the sum of the responses and range from 0 to 52. Higher scores indicate better quality of life (QoL). In aspects, a change from baseline in (FACIT)-Fatigue may be determined. In aspects, a method wherein a subject is administered a Tn3 scaffold has a higher (FACIT)-Fatigue Scale as compared to a comparable method wherein the subject is not administered a Tn3 scaffold.

In aspects, a FACIT-Fatigue Scale assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a FACIT-Fatigue Scale assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a FACIT-Fatigue assessment can occur at any point before, during, or after administration with a Tn3 scaffold.

### Ocular Surface Disease Index (OSDI©)

In aspects, an assessment may comprise an OSDI. OSDI is a valid and reliable instrument for assessing effect on vision-related function and dry eye disease severity (normal, mild, moderate, and severe). An OSDI recall period is 1 week. The OSDI assessment may be composed of 12 questions that a physician asks a subject and circles the number that best represents each question. Responses for each question range from 0 (None of the time) to 4 (All of the time). An OSDI score is calculated as (sum of scores for questions answered)/(number of questions answered)×25, which ranges from 0 to 100 with higher scores signifying greater disability. In aspects, a change from baseline in OSDI is determined.

In aspects, an OSDI assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, an OSDI assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, an OSDI assessment can occur at any point before, during, or after administration with a Tn3 scaffold.

### Patient Global Impression of Severity (PGIS)

In aspects, an assessment comprises completing a PGIS. A PGIS is a single item designed to capture a subject's perception of overall symptom severity over the past week on a 5-point categorical response scale (none, mild, moderate, severe, or very severe). In aspects, a PGIS score is reduced following administration of a Tn3 scaffold of the disclosure. A reduction can be of from about 1, 2, 3, 4, or up to about 5 points.

In aspects, a PGIS assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a PGIS assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a PGIS assessment can occur at any point before, during, or after administration with a Tn3 scaffold.

### Physician's Global Impression of Severity

In aspects, an assessment may comprise a Physician's Global Impression of Severity (MDGIS). A Physician's Global Impression of Severity may comprise an overall assessment of SS disease severity or severity of an autoimmune disease of the disclosure. A PGIS may be scored on a 5-point categorical response scale (none, mild, moderate, severe, or very severe). In aspects, a change from baseline in the Physician's Global Impression of Severity is determined. In aspects, a Physician's Global Impression of Severity score is reduced following administration of a Tn3 scaffold of the disclosure. A reduction can be of from about 1, 2, 3, 4, or up to about 5 points.

In aspects, a Physician's Global Impression of Severity assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a Physician's Global Impression of Severity assessment to a Tn3 scaffold can be assessed on about Day 1, Day 15±1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a MDGIS assessment can occur at any point before, during, or after administration with a Tn3 scaffold.

### Composite Index

In aspects, an assessment comprises determining composite index. A composite index may be defined as either ESSDAI-3 improvement (i.e., a decrease of 3 points) or ESSPRI-1 improvement (i.e., ≥1 point or 15% reduction from baseline in ESSPRI score) plus no worsening of Physician Global Impression of Severity from baseline.

In aspects, a Composite Index assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a Composite Index assessment to a Tn3 scaffold can be assessed on about Day 85±3, Day 169±3, and Day 309±7, post treatment initiation. In aspects, a composite index assessment can occur at any point before, during, or after administration with a Tn3 scaffold.

### 28-Joint Assessment

In aspects, an assessment may comprise a 28-Joint Count. A 28-Joint Count may assess the following joints for tenderness and swelling: left and right shoulder, elbow, wrist, metacarpophalangeal (MCP)1, MCP2, MCP3, MCP4, MCP5, proximal interphalangeal (PIP)1, PIP2, PIP3, PIP4, PIP5 joints of the upper extremities, and left and right knee of the lower extremities. Each of the 28 joints may be evaluated for the presence of synovitis. At the start of the 28-joint count (prior to assessment of tenderness and swelling) a subject may be asked if they have experienced or are experiencing pain in any of the 28 joints. In aspects, an assessment may comprise a tender joint count (TJC). In aspects, an assessment may comprise a swollen joint count (SJC).

In aspects, a 28-Joint Count assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a 28-Joint Count assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a 28-Joint Count assessment can occur at any point before, during, or after administration with a Tn3 scaffold.

### Salivary and Lacrimal Function

### Total Stimulated Salivary Flow

In aspects, an assessment comprises determining total stimulated salivary flow. Whole stimulated salivary flow may be measured to objectively assess functional changes in the salivary glands. Subjects receiving standard of care for xerostomia at screening must discontinue use of pilocarpine or cevimeline for at least 12 hours and artificial saliva for at least 3 hours prior to the collection of saliva. Subjects should be prohibited from eating or drinking for at least 90 minutes prior to the collection of saliva. To minimize diurnal variation, every attempt should be made to collect saliva at the same time of the day across all subsequent assessments.

In an exemplary whole stimulated salivary flow rate measurement, an approximately 5×5 cm parafilm square is rolled and given to a subject to be chewed like a gum at a rate of approximately 60 strokes per minute. The subject should be seated upright with eyes open, and head tilted slightly forward and start chewing the parafilm for 60 seconds, at which point all the collected saliva should be spit in an extra (not pre-weighed) falcon tube, keeping the parafilm in the mouth. This first collection accustoms the subject in the procedure. Three rounds of salivary collection, each after 20-second chewing, follow in a pre-weighed falcon tube. Those rounds are continuous, but timing should be stopped during the collection of the saliva and restart immediately after the saliva deposition in a pre-weighed falcon tube, for a total stimulation time of 60 seconds. If collection for 60 seconds is not feasible due to subjects' inability, the total collection time should be recorded. Total stimulated saliva collected is assessed by subtracting the final weight of the tube from the weight prior to collection. In aspects, a change from baseline in Total Stimulated Salivary Flow is determined. In aspects, a post-treatment unstimulated whole saliva flow rate>0.1 ml/min in subjects in population #1.

In aspects, a stimulated salivary flow assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a stimulated salivary flow assessment to a Tn3 scaffold can be assessed on about Day 1, Day 85±3, Day 169±3, Day 253±3, Day 309±7, and Day 365±7 post treatment initiation. In aspects, a stimulated salivary slow assessment can occur at any point before, during, or after administration with a Tn3 scaffold.

### Visual Analog Scale (VAS) Oral/Ocular

In aspects, an assessment comprises determining (VAS) Oral/Ocular. These 2 instruments use a continuous 100 mm VAS, from 0 m (best) to 100 mm (worst), to assess change in the severity of oral and ocular dryness. The respondent is asked to place a line perpendicular to the VAS line at the point that represents the symptom intensity. The VAS Oral may rate the question “How dry your mouth feels most of the time” (not dry at all 0 mm; very dry 100 mm). The VAS Ocular may rate the question “How dry do your eyes feel most of the time” (not dry at all 0 mm; very dry 100 mm). In aspects, a baseline (VAS) Oral/Ocular score is reduced by about 3%, 5%, 10%, 20%, 30%, 40%, 50%, 60%, 70%, 80%, 90%, or up to about 100% as compared to a subject's VAS score prior to administration of a Tn3 scaffold.

In aspects, a VAS assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a VAS assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a VAS assessment can occur at any point before, during, or after administration with a Tn3 scaffold.

### Schirmer's Test

In aspects, a Schirmer's test without local anesthesia may be performed. Schirmer's test measures lacrimal gland function. A Schirmer's test may use calibrated strips of a non-toxic filter paper to measure the flow of tears. One end of the strip is placed within the lower eyelid. Both eyes need to be measured simultaneously. After the placement, subjects are asked to keep their eyes gently closed for 5 minutes at which points the strips are removed from the eyelids and the extent of the wetting of each of the strips is recorded. In aspects, a change from baseline in Schirmer's test may be determined. In aspects, a post-treatment score is >5 mm/5 min on at least one eye.

In aspects, a Schirmer's test assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a Schirmer's test assessment to a Tn3 scaffold can be assessed on about Day 1, Day 85±3, Day 169±3, Day 253±3, Day 309±7, and Day 365±7 post treatment initiation. In aspects, a Schirmer's test assessment can occur at any point before, during, or after administration with a Tn3 scaffold.

### Subjective Complaints

### Patient Global Impression of Change (PGIC)

In aspects, an assessment comprises a PGIC. A PGIC is a single item designed to capture the subject's perception of change in their overall symptom severity from starting the study medication. Change in severity is captured using a 5-point scale (much better, a little better, no change, a little worse, or much worse). In aspects, a change from baseline in PGIC is determined.

In aspects, a PGIC assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a PGIC assessment to a Tn3 scaffold can be assessed on about Day 15±1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a PGIC assessment can occur at any point before, during, or after administration with a Tn3 scaffold.

### 36-Item Short Form Survey Version 2 (SF36v2) Physical Component Score and Mental Component Score

In aspects, an assessment comprises a SF-36v2 profile. A SF-36v2 (acute recall) is a 36-item general health status assessment that captures information about 8 health domains: Physical Functioning, Role Physical, Bodily Pain, General Health, Vitality, Social Functioning, Role Emotional, and Mental Health. The SF-36v2 provides scores for each domain as well as 2 psychometrically-based summary scores: physical component score and mental component score. The recall period for the acute version is one week (i.e., “last week”). In aspects, a change from baseline in SF36v2 Physical Component Score and Mental Component Score is determined.

In aspects, a 36v2 profile assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a 36v2 profile assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a SF36v2 assessment can occur at any point before, during, or after administration with a Tn3 scaffold.

### Patient-Reported Outcomes Measurement Information System® (PROMIS-29 Profile)

In aspects, an assessment comprises a PROMIS-29 profile. A PROMIS-29 assesses 7 domains (Depression, Anxiety, Physical Function, Pain Interference, Fatigue, Sleep Disturbance, and Ability to Participate in Social Roles and Activities) with 4 questions on each domain. The assessment of the domains is for the past 7 days except for Physical Function which has no timeframe specified. In aspects, a treated subject has an improved score on a PROMIS-29 as compared to an otherwise comparable method wherein the subject of the otherwise comparable method is not administered a Tn3 scaffold.

In aspects, a PROMIS-29 profile assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a PROMIS-29 profile assessment to a Tn3 scaffold can be assessed on about Day 1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a PROMIS-29 profile assessment can occur at any point before, during, or after administration with a Tn3 scaffold.

### Pharmacokinetic (PK) Analysis

In aspects, a method provided herein can comprise determining a concentration of a Tn3 scaffold in a subject in need thereof post administration. In aspects, a method comprises a pharmacokinetic assessment. In aspects, a sample is a blood sample or a plasma sample, or a combination of both. In aspects, a suitable assay to measure pharmacokinetics may comprise electrochemiluminescence (ECL) assay, a bead-based assay, a cell-based assay, and combinations thereof. In aspects, a sample may comprise plasma and the plasma is assessed for a Tn3 scaffold concentration by measuring: maximum observed concentration (Cₘₐₓ), area under the concentration-time curve (AUC), CL, and terminal elimination half-life (t_{1/2}).

In aspects, a PK assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a PK assessment to a Tn3 scaffold can be assessed on about Day 1, Day 15±1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, a PK assessment can occur at any point before, during, or after administration with a Tn3 scaffold.

### Immunogenicity

In aspects, an assessment comprises determining a level of immunogenicity, if any, of a Tn3 scaffold. Immunogenicity comprises determining the presence of anti-drug antibodies (ADA) to a Tn3 scaffold. The presence of ADA can be evaluated using a plasma sample from a subject administered a Tn3 scaffold. In aspects, ADA are not detected post administration of a Tn3 scaffold. In aspects, ADA levels are reduced as compared to an otherwise comparable method wherein the subject of the otherwise comparable method is not administered a Tn3 scaffold; for example, the reduction may be about: 10%, 15%, 20%, 25%, 50%, 60%, 70%, 80%, 90%, 95%, 96%, 97%, 98%, 99%, or up to 100% as compared to ADA levels in an otherwise comparable method wherein the subject of the otherwise comparable method is not administered a Tn3 scaffold.

In aspects, an immunogenicity assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, immunogenicity assessment to a Tn3 scaffold can be assessed on about Day 1, Day 29±3, Day 85±3, Day 169±3, Day 197±3, Day 253±3, Day 309±7, and Day 365±7 post treatment initiation. In aspects, an immunogenicity assessment can occur at any point before, during, or after administration with a Tn3 scaffold.

### Pharmacodynamics and Biomarker Analysis

In aspects, a method comprises a pharmacodynamic assessment. In aspects, an assessment comprises determining the presence or absence of biomarkers, as well any changes from baseline post treatment with a composition of the disclosure: T-cell subsets, B-cell subsets, SS-A, SS-B, and IgG and IgM RF autoantibodies and/or markers of inflammation (immunoglobulins, beta-2 microglobulin, CRP, C3, C4, and serum free light chains). In aspects, serum and plasma may be collected to measure changes in exploratory biomarkers of disease activity such as proinflammatory cytokines, and CXCL13 levels. In aspects, biomarkers associated with B-cell subsets, RF autoantibodies, and CXCL13 are analyzed. In aspects, erythrocyte sedimentation rate (ESR) is analyzed. In aspects, an analysis of biomarkers may include methods such as flow cytometry or mass spectrometry (e.g., LC-MS), or the like, to evaluate changes in T and B-cell subsets, and salivary proteins, and changes in fecal microbiome. Exemplary B and T cell subsets include, but are not limited to, plasmablasts (e.g., CD27^{br}/CD38^{br}/IgD− subsets of CD19+ cells), precursor memory B cells (e.g., CD11c^{br} subsets of CD19+ cells), T follicular helper (Tfh) cells (e.g., CXCR5+/ICOS+ subsets of CD3+/CD4+ cells), proliferation of post-switch memory B cells (e.g., Ki67+ subsets of CD27^{br}/IgD−/CD19+ cells), and/or proliferation of total T-cells (e.g., Ki67+ subsets of CD3+ cells).

In aspects, a sample may be evaluated for protein biomarkers. Exemplary protein biomarkers include, but are not limited to, TNFRSF13B, BAFF, CXCL10, CXCL12, IL6, CCL21, CXCL9, IL12B, TNFRSF4, LAMP3, CD22, CD79B, SLAMF7, IL10, TNFRSF8, CXCL11, CCL19, CXCL13, TNFRSF9, TNFRSF6B, NOS3, CD72, ADA2, TL2RA, CD27, PDCD1, IL12A, IL12B, LAG3, ADGRE1, TIGIT, FCAMR, CD80, or any combination thereof. In aspects, a protein biomarker is TNFRSF13B. In aspects, a protein biomarker is BAFF. In aspects, a protein biomarker is CXCL10. In aspects, a protein biomarker is CXCL12. In aspects, a protein biomarker is IL6. In aspects, a protein biomarker is a protein biomarker is CCL21. In aspects, a protein biomarker is CXCL9. In aspects, a protein biomarker is IL12B. In aspects, a protein biomarker is TNFRSF4. In aspects, a protein biomarker is LAMP3. In aspects, a protein biomarker is CD22. In aspects, a protein biomarker is CD79B. In aspects, a protein biomarker is SLAMF7. In aspects, a protein biomarker is IL10. In aspects, a protein biomarker is TNFRSF8. In aspects, a protein biomarker is CXCL11. In aspects, a protein biomarker is CCL19. In aspects, a protein biomarker is CXCL13. In aspects, a protein biomarker is TNFRSF9. In aspects, a protein biomarker is TNFRSF6B. In aspects, a protein biomarker is NOS3. In aspects, a protein biomarker is CD72. In aspects, a protein biomarker is ADA2. In aspects, a protein biomarker is IL2RA. In aspects, a protein biomarker is CD27. In aspects, a protein biomarker is PDCD1. In aspects, a protein biomarker is IL12A. In aspects, a protein biomarker is IL12B. In aspects, a protein biomarker is LAG3. In aspects, a protein biomarker is ADGRE1. In aspects, a protein biomarker is TIGIT. In aspects, a protein biomarker is FCAMR. In aspects, a protein biomarker is CD80.

In aspects, a sample evaluated for a protein biomarker is acquired from a subject. In aspects, a subject has symptoms of or has been diagnosed with a disease or disorder. In aspects, a subject has symptoms of or has been diagnosed with an autoimmune disease of the disclosure (e.g., Sjögren's syndrome, rheumatoid arthritis, etc.). In aspects, a subject has symptoms of or has been diagnosed with Sjögren's syndrome. In aspects, a subject has symptoms of or has been diagnosed with rheumatoid arthritis.

In aspects, a level of a protein biomarker is increased or decreased in sample acquired from a subject that has symptoms of or has been diagnosed with an autoimmune disease (e.g., Sjögren's syndrome, rheumatoid arthritis) as compared to healthy subject. In aspects, a level of a protein biomarker is increased or decreased in a subject that has symptoms of or has been diagnosed with an autoimmune disease (e.g., Sjögren's syndrome, rheumatoid arthritis) after administration of a compound or composition of the disclosure as compared to an equivalent subject not administered the compound or composition.

In aspects, a level of a protein biomarker is increased in sample acquired from a subject that has symptoms of or has been diagnosed with an autoimmune disease (e.g., Sjögren's syndrome, rheumatoid arthritis) as compared to healthy subject. In aspects, a level of a protein biomarker is increased in a subject that has symptoms of or has been diagnosed with an autoimmune disease (e.g., Sjögren's syndrome, rheumatoid arthritis) after administration of a compound or composition of the disclosure as compared to an equivalent subject not administered the compound or composition. Exemplary protein biomarkers that are increased include, but are not limited to, TNFRSF13B, BAFF, CXCL10, CXCL12, IL6, CCL21, CXCL9, IL12B, TNFRSF4, LAMP3, CD22, CD79B, SLAMF7, IL10, TNFRSF8, CXCL11, CCL19, CXCL13, TNFRSF9, TNFRSF6B, NOS3, CD72, ADA2, IL2RA, CD27, PDCD1, IL12A, IL12B, LAG3, ADGRE1, TIGIT, FCAMR, CD80, or any combination thereof. In aspects, a protein biomarker that is increased is TNFRSF13B. In aspects, a protein biomarker that is increased is BAFF. In aspects, a protein biomarker that is increased is CXCL10. In aspects, a protein biomarker that is increased is CXCL12. In aspects, a protein biomarker that is increased is IL6. In aspects, a protein biomarker that is increased is a protein biomarker that is increased is CCL21. In aspects, a protein biomarker that is increased is CXCL9. In aspects, a protein biomarker that is increased is IL12B. In aspects, a protein biomarker that is increased is TNFRSF4. In aspects, a protein biomarker that is increased is LAMP3. In aspects, a protein biomarker that is increased is CD22. In aspects, a protein biomarker that is increased is CD79B. In aspects, a protein biomarker that is increased is SLAMF7. In aspects, a protein biomarker that is increased is IL10. In aspects, a protein biomarker that is increased is TNFRSF8. In aspects, a protein biomarker that is increased is CXCL11. In aspects, a protein biomarker that is increased is CCL19. In aspects, a protein biomarker that is increased is CXCL13. In aspects, a protein biomarker that is increased is TNFRSF9. In aspects, a protein biomarker that is increased is TNFRSF6B. In aspects, a protein biomarker that is increased is NOS3. In aspects, a protein biomarker that is increased is CD72. In aspects, a protein biomarker that is increased is ADA2. In aspects, a protein biomarker that is increased is IL2RA. In aspects, a protein biomarker that is increased is CD27. In aspects, a protein biomarker that is increased is PDCD1. In aspects, a protein biomarker that is increased is IL12A. In aspects, a protein biomarker that is increased is IL12B. In aspects, a protein biomarker that is increased is LAG3. In aspects, a protein biomarker that is increased is ADGRE1. In aspects, a protein biomarker that is increased is TIGIT. In aspects, a protein biomarker that is increased is FCAMR. In aspects, a protein biomarker that is increased is CD80.

In aspects, a level of a protein biomarker is decreased in sample acquired from a subject that has symptoms of or has been diagnosed with an autoimmune disease (e.g., Sjögren's syndrome, rheumatoid arthritis) as compared to healthy subject. In aspects, a level of a protein biomarker is decreased in a subject that has symptoms of or has been diagnosed with an autoimmune disease (e.g., Sjögren's syndrome, rheumatoid arthritis) after administration of a compound or composition of the disclosure as compared to an equivalent subject not administered the compound or composition. Exemplary protein biomarkers that are decreased include, but are not limited to, TNFRSF13B, BAFF, CXCL10, CXCL12, IL6, CCL21, CXCL9, IL12B, TNFRSF4, LAMP3, CD22, CD79B, SLAMF7, IL10, TNFRSF8, CXCL11, CCL19, CXCL13, TNFRSF9, TNFRSF6B, NOS3, CD72, ADA2, IL2RA, CD27, PDCD1, IL12A, IL12B, LAG3, ADGRE1, TIGIT, FCAMR, CD80, or any combination thereof. In aspects, a protein biomarker that is decreased is TNFRSF13B. In aspects, a protein biomarker that is decreased is BAFF. In aspects, a protein biomarker that is decreased is CXCL10. In aspects, a protein biomarker that is decreased is CXCL12. In aspects, a protein biomarker that is decreased is IL6. In aspects, a protein biomarker that is decreased is a protein biomarker that is decreased is CCL21. In aspects, a protein biomarker that is decreased is CXCL9. In aspects, a protein biomarker that is decreased is IL12B. In aspects, a protein biomarker that is decreased is TNFRSF4. In aspects, a protein biomarker that is decreased is LAMP3. In aspects, a protein biomarker that is decreased is CD22. In aspects, a protein biomarker that is decreased is CD79B. In aspects, a protein biomarker that is decreased is SLAMF7. In aspects, a protein biomarker that is decreased is IL10. In aspects, a protein biomarker that is decreased is TNFRSF8. In aspects, a protein biomarker that is decreased is CXCL11. In aspects, a protein biomarker that is decreased is CCL19. In aspects, a protein biomarker that is decreased is CXCL13. In aspects, a protein biomarker that is decreased is TNFRSF9. In aspects, a protein biomarker that is decreased is TNFRSF6B. In aspects, a protein biomarker that is decreased is NOS3. In aspects, a protein biomarker that is decreased is CD72. In aspects, a protein biomarker that is decreased is ADA2. In aspects, a protein biomarker that is decreased is IL2RA. In aspects, a protein biomarker that is decreased is CD27. In aspects, a protein biomarker that is decreased is PDCD1. In aspects, a protein biomarker that is decreased is IL12A. In aspects, a protein biomarker that is decreased is IL12B. In aspects, a protein biomarker that is decreased is LAG3. In aspects, a protein biomarker that is decreased is ADGRE1. In aspects, a protein biomarker that is decreased is TIGIT. In aspects, a protein biomarker that is decreased is FCAMR. In aspects, a protein biomarker that is decreased is CD80.

In aspects, flow cytometry is utilized to evaluate cellular markers. In general, positive staining can be categorized as dim, moderate, bright or variable. Dim staining can be slightly increased when compared to the negative control. Moderate staining can be at least one log decade brighter than the negative control. Bright staining can be at least two log decades brighter than the negative control. In aspects, the terms bright and high can be used interchangeably herein.

In aspects, B-cell subsets are evaluated. In aspects, B-cell subsets that are evaluated are selected from the group consisting of: transitional, naive, plasma, memory, and any combination of B-cell subsets thereof. In aspects, a B-cell subset comprises transitional cells. In aspects, a B-cell subset comprises naïve cells. In aspects, a B-cell subset comprise plasma cells. In aspects, a B-cell subset comprises memory cells. In aspects, B-cell subsets that are evaluated are plasma and memory cells. In aspects, the B-cell subsets comprise Ki67+CD27+ memory cells, CD27 high CD38 high plasmablasts, and CD11c high atypical memory cells. In aspects, the B-cell subsets comprise Ki67+ subsets of CD19+ cells. In aspects, the B-cell subsets comprise CD86+ subsets of CD19+ cells. In aspects, the B-cell subsets comprise CD86+ subsets of CD27+/IgD−/CD19+ cells. In aspects, the B-cell subsets comprise Ki67+ subsets of CD27+/IgD−/CD19+ cells. In aspects, the B-cell subsets comprise CD27^{br}/IgD−/CD38^{br} subsets of CD19+ cells. In aspects, the B-cell subsets comprise Ki67+ subsets of CD27+/IgD−/CD19+ cells. In aspects, the B cell subsets comprise CD11c^{br} subsets of CD19+ cells. In aspects, the B-cell subsets comprise CD86+ subsets of CD11c^{br}/CD19+ cells.

In aspects, a B-cell subset comprises transitional cells that comprise any combination of biomarkers selected from the group consisting of: CD19+, CD20+, CD27−, CD10+, CD24high, CD38high, IgD+, CD5+, and BR3+. In aspects, a B-cell subset comprises mature naïve cells that comprise any combination of biomarkers selected from the group consisting of: CD19+, CD20+, CD27, CD24int, CD38int, CD21+, CD10−, IgD+, and IgM−. In aspects, a B-cell subset comprises germinal center cells that comprise any combination of biomarkers selected from the group consisting of: CD19+, CD10+, CD20+, CD27+, CD38+, FAS+, and IgDBR3+. In aspects, a B-cell subset comprises memory cells that comprise any combination of biomarkers selected from the group consisting of: CD19+, CD20+, CD27+, and CD38. In aspects, a B-cell subset comprises plasmablasts that comprise any combination of biomarkers selected from the group consisting of: CD19+, CD20, CD27+, CD38high, IgD, and CD138−. In aspects, a B-cell subset comprises plasma cells that comprise any combination of biomarkers selected from the group consisting of: CD19+, CD20−, IgD, CD27high, CD38high, CD24lo, CD138high, and TACI+. In aspects, B-cells are identified according to the presence or absence of any combination of markers selected from the group comprising: CD10, CD11c, CD19, CD20, CD21, CD24, CD27, CD38, CD138, Ki67, TACI+, IgM, or IgD.

In aspects T-cell subsets are evaluated. In aspects, CD4+ subsets are evaluated, for example, Th1, Th2, Th9, Th17, Th22, Treg, or Tfh, or combinations thereof. In aspects, CD4+Th1 subsets are evaluated. In aspects, CD4+ Th2 subsets are evaluated. In aspects, CD4+ Th9 subsets are evaluated. In aspects, CD4+ Th17 subsets are evaluated. In aspects, CD4+ Th22 subsets are evaluated. In aspects, CD4+ Treg subsets are evaluated. In aspects, CD4+ Tfh subsets are evaluated. In aspects, CD8+ memory and effector subsets are evaluated (CD25, CD45RO, CD45RA, CCR-7, L-Selectin [CD62L], and/or intracellular marker (FOXP3). In aspects, Naïve CD4+ T cells are evaluated that comprise any combination of markers selected from the group consisting of: CD4+, CD45RA+, CD45RO−, and CCR7+. In aspects, Th1 cells are evaluated that comprise any combination of biomarkers selected from the group consisting of: CD4+, CXCR3+, CCR5+, and IL12RP2+. In aspects, Th2 cells are evaluated that comprise any combination of biomarkers selected from the group consisting of: CD4+, CCR4+, and IL12RP2−. In aspects, Th9 cells are evaluated that comprise any combination of biomarkers selected from the group consisting of: CD4+, CCR3+, and CCR6+. In aspects, Th17 cells are evaluated that comprise any combination of biomarkers selected from the group consisting of: CD4+, CCR6+, CCR4+, and NK1.1+. In aspects, Th22 cells are evaluated that comprise any combination of biomarkers selected from the group consisting of: CD4+, CCR10+, CCR4+, and CCR6+. In aspects, Treg cells are evaluated that comprise any combination of biomarkers selected from the group consisting of: CD4+, CD127+, CD25+, and CTLA-4+. In aspects, Tfh cells are evaluated that comprise any combination of biomarkers selected from the group consisting of: CD3+, CD4+, CXCR5+, CD40L+, and ICOS+. In aspects, γδ T cells are evaluated that comprise any combination of biomarkers selected from the group consisting of: TCRγ/δ+, and IL23R+. In aspects, the T-cell subsets comprise Ki67+ subsets of CD3+ cells. In aspects, the T-cell subsets comprise Ki67+ cells of CD3+ cells. In aspects, the T-cell subsets comprise Ki67+ cells of CD3+/CD4+ cells. In aspects, the T-cell subsets comprise Ki67+ subsets of CD3+/CD4+/CD45RA−/CCRT7+ cells. In aspects, the T-cell subsets comprise Ki67+ subsets of CD3+/CD4+/CD45RA−/CCR7− cells. In aspects, the T-cell subsets comprise Ki67+ subsets of CD3+/CD4+/CXCR5+/ICOS+ cells. In aspects, the T-cell subsets comprise Ki67+ subsets of CD3+/CD8+ cells. In aspects, the T-cell subsets comprise Ki67+ subsets of CD3+/CD8+/CD45RA−/CCR7+ cells. In aspects, the T-cell subsets comprise Ki67+ subsets of CD3+/CD8+/CD45RA/CCR7−cells. In aspects, the T-cell subsets comprise Ki67+ subsets of CD3+/CD8+/CXCR5+ cells. In aspects, the Tfh subsets comprise CXCR5+/ICOS+ subsets of CD3+/CD4+ cells. In aspects, NKT cells are evaluated that comprise any combination of biomarkers selected from the group consisting of: TCR Vα24+, and TCR Vβ11+. In aspects, Naïve CD8+ T cells are evaluated that comprise any combination of biomarkers selected from the group consisting of: CD8+, CD45RA+, CD45RO−, CCR7+, and CD28+. In aspects, cytotoxic CD8+ T cells are evaluated that comprise any combination of biomarkers selected from the group consisting of: CD8+, and CCR7−.

In aspects, the plasmablast subset comprises CD27c^{br}/CD38^{br}/IgD− of CD19+ cells.

In aspects, dendritic cells (DCs) are evaluated. In aspects, the DCs are plasmacytoid DCs (pDCs). In aspects, the DCs are circulating pDCs. In aspects, DC subsets are evaluated for one or more markers. In aspects a marker is LAMP3.

In aspects, a whole blood sample may be collected for the assessment of changes in the number, activation status, and frequency of major leukocyte populations, including B and T lymphocytes using flow cytometry.

In aspects, administration of a Tn3 scaffold of the disclosure reduces the level of certain B-cell and plasmablast/plasma-cell populations. In aspects, administration of a Tn3 scaffold reduces the level of T cells and/or other immune cell populations.

In aspects, a Tn3 scaffold may achieve at least about 10%, 15%, 20%, 25%, 50%, 60%, 70%, 80%, 90%, 95%, 96%, 97%, 98%, 99%, or up to 100% reduction in biomarker levels in a subject administered the Tn3 scaffold as compared to the subject at baseline or a subject not treated with the Tn3 scaffold. In aspects, a Tn3 scaffold may achieve at least about 10%, 15%, 20%, 25%, 50%, 60%, 70%, 80%, 90%, 95%, 96%, 97%, 98%, 99%, or up to 100% reduction in one or more protein biomarker levels in a subject administered the Tn3 scaffold as compared to the subject at baseline or a subject not treated with the Tn3 scaffold. In aspects, a Tn3 scaffold may achieve at least about 10%, 15%, 20%, 25%, 50%, 60%, 70%, 80%, 90%, 95%, 96%, 97%, 98%, 99%, or up to 100% reduction in plasmablast subsets of the disclosure in a subject administered the Tn3 scaffold as compared to the subject at baseline or a subject not treated with the Tn3 scaffold.

In aspects, a Tn3 scaffold may achieve at least about 10%, 15%, 20%, 25%, 50%, 60%, 70%, 80%, 90%, 95%, 96%, 97%, 98%, 99%, or up to 100% reduction in one or more B-cell subsets of the disclosure in a subject administered the Tn3 scaffold as compared to the subject at baseline or a subject not treated with the Tn3 scaffold. In aspects, a Tn3 scaffold may achieve at least about 10%, 15%, 20%, 25%, 50%, 60%, 70%, 80%, 90%, 95%, 96%, 97%, 98%, 99%, or up to 100% reduction in one or more post-switch memory B-cell subsets of the disclosure in a subject administered the Tn3 scaffold as compared to the subject at baseline or a subject not treated with the Tn3 scaffold. In aspects, a Tn3 scaffold may achieve at least about 10%, 15%, 20%, 25%, 50%, 60%, 70%, 80%, 90%, 95%, 96%, 97%, 98%, 99%, or up to 100% reduction in one or more precursor memory B-cell subsets of the disclosure in a subject administered the Tn3 scaffold as compared to the subject at baseline or a subject not treated with the Tn3 scaffold.

In aspects, a Tn3 scaffold may achieve at least about 10%, 15%, 20%, 25%, 50%, 60%, 70%, 80%, 90%, 95%, 96%, 97%, 98%, 99%, or up to 100% reduction in one or more T-cell subsets of the disclosure in a subject administered the Tn3 scaffold as compared to the subject at baseline or a subject not treated with the Tn3 scaffold. In aspects, a Tn3 scaffold may achieve at least about 10%, 15%, 20%, 25%, 50%, 60%, 70%, 80%, 90%, 95%, 96%, 97%, 98%, 99%, or up to 100% increase in one or more Tfh subsets in a subject administered the Tn3 scaffold as compared to the subject at baseline or a subject not treated with the Tn3 scaffold.

In aspects, a Tn3 scaffold may achieve at least about 10%, 15%, 20%, 25%, 50%, 60%, 70%, 80%, 90%, 95%, 96%, 97%, 98%, 99%, or up to 100% reduction in plasmablast subsets of the disclosure in a subject administered the Tn3 scaffold as compared to the subject at baseline or a subject not treated with the Tn3 scaffold.

In aspects, a pharmacodynamic assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a pharmacodynamic assessment to a Tn3 scaffold can be assessed on about Day 1, Day 15±1, Day 29±3, Day 57±3, Day 85±3, Day 113±3, Day 141±3, Day 169±3, Day 197±3, Day 225±3, Day 253±3, Day 281±3, Day 309±7, and Day 365±7 posttreatment initiation. In aspects, a pharmacodynamic assessment can occur at any point before, during, or after administration with a Tn3 scaffold.

### Fecal Microbiome Sample

In aspects, an assessment may comprise changes in fecal microbiome. In aspects, stool specimens from whole stools may be collected to support assessment of changes over time in a composition of the gut microbiome by 16S microarray and/or deep sequencing methods. In aspects, a self-collection microbial DNA collection kit from feces is provided prior to or during visits to collect and test the fecal sample. In aspects, a change from baseline in fecal microbiome is determined.

In aspects, a fecal microbiome assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a fecal microbiome assessment to a Tn3 scaffold can be assessed on about Day 1, Day 169±3, and Day 309±7 post treatment initiation. In aspects, a fecal microbiome assessment can occur at any point before, during, or after administration with a Tn3 scaffold.

### Autoantibodies

In aspects, an assessment comprises determining a level of autoantibodies in a subject. Autoantibodies comprise those that react with self-antigens. Exemplary autoantibodies comprise SS-A, SS-B and IgG and/or IgM rheumatoid factor (RF) autoantibodies and combinations thereof. In aspects, serum is collected to assess the presence of anti-SSA (Ro), anti-SSB (La), antinuclear antibodies, and RF. Exemplary methods of evaluating levels of autoantibodies comprise EliA immunoassay, microarray, ELISA, or combinations thereof. In aspects, autoantibodies are not detected post administration of a Tn3 scaffold. In aspects, treatment comprises reducing autoantibodies in a subject by about: 20%, 30%, 40%, 45%, 50%, 60%, 75%, 80%, 90%, 95%, 96%, 97%, 98%, 99%, or up to 100% as compared to of the levels of autoantibodies in an otherwise comparable method wherein the subject of the otherwise comparable method is not administered a Tn3 scaffold. In aspects, a change from baseline in SS-A, SS-B and IgG and/or IgM rheumatoid factor (RF) autoantibodies is determined. In aspects, autoantibodies are detected at levels of at most about: 1-fold, 2-fold, 3-fold, 4-fold, 5-fold, 10-fold, 15-fold, 20-fold, 25-fold, 30-fold, 35-fold, 40-fold, 45-fold, or 50-fold as compared to an otherwise comparable method wherein the subject of the otherwise comparable method is not administered a Tn3 scaffold. In aspects, administration of a Tn3 scaffold of the disclosure is effective in reducing a level of autoantibodies in a subject by at least about or at most about: 3%-5%, 5%-10%, 10%-20%, or 5%-25% as compared to a baseline level prior to the administration. In aspects, administration of a Tn3 scaffold is effective in eliminating autoantibodies in the subject in need.

In aspects, an auto-antibody assessment to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, an auto-antibodies assessment to a Tn3 scaffold can be assessed on about Day 1, Day 85±3, Day 169±3, and Day 253±3 post treatment initiation. In aspects, an auto-antibodies assessment can occur at any point before, during, or after administration with a Tn3 scaffold.

### Markers of Inflammation

In aspects, an assessment comprises determining a level of markers of inflammation. Exemplary markers of inflammation include but are not limited to: immunoglobulins (IgM, IgG, IgA), beta-2 microglobulin, C-reactive protein (CRP), CXCL13, C3, C4 and serum free light chains, cryoglobulins, and serum and urine immunofixation and combinations thereof. In aspects, whole blood, plasma, serum, and urine is collected to assess markers of inflammation. In aspects, a change as compared to a baseline is determined. In aspects, a change from baseline in levels of markers of inflammation (immunoglobulins, beta-2 microglobulin, C-reactive protein [CRP], CXCL13, C3, C4 and serum free light chains) is determined. In aspects, suitable assays to assess a level of inflammation comprise: ELISA, hs-CRP test, CRP test, Luminex, and combinations thereof. In aspects, administration of a Tn3 scaffold of the disclosure is effective in reducing a level of a biomarker of inflammation in a subject by at least about or at most about: 20%, 30%, 40%, 45%, 50%, 60%, 75%, 80%, 90%, 95%, 96%, 97%, 98%, 99%, or up to 100% as compared to of the levels of autoantibodies in an otherwise comparable method wherein the subject of the otherwise comparable method is not administered a Tn3 scaffold In aspects, administration of a Tn3 scaffold of the disclosure is effective in reducing a level of a biomarker of inflammation in a subject by at least about or at most about: 3%-5%, 5%-10%, 10%-20%, or 5%-25% as compared to a baseline level prior to the administration.

In aspects, an assessment of markers of inflammation to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, an assessment of markers of inflammation to a Tn3 scaffold can be assessed on about Day 1, Day 15±1, Day 29±3, Day 57±3, Day 85±3, Day 113±3, Day 141±3, Day 169±3, Day 197±3, Day 225±3, Day 253±3, Day 281±3, Day 309±7, and Day 365±7 post treatment initiation. In aspects, an assessment of markers of inflammation can occur at any point before, during, or after administration with a Tn3 scaffold.

### RNA and DNA Analysis

In aspects, an assessment comprises quantifying expression levels of genes associated with disease activity by way of RNA and/or DNA analysis. In aspects, RNA testing is performed. In aspects, RNA testing is performed to measure expression levels of genes associated with disease activity, specific cell types including plasma cell gene signature, T follicular helper gene signature, and signaling, including the CD40L/CD40 pathway.

In aspects, blood RNA is used to measure the expression levels of genes associated with disease activity, specific cell types including plasma cell gene signature, T follicular helper gene signature, and signaling, including the CD40L/CD40 pathway. In aspects, a blood sample will be collected using the PAXgene Blood RNA System for the collection, transport, and storage of blood and stabilization of intracellular RNA in a closed tube and subsequent isolation and purification of intracellular RNA from whole blood for microarray analysis and quantitative polymerase chain reaction.

In aspects, DNA testing may be performed. DNA may be isolated at baseline from blood for pharmacogenomic (single nucleotide polymorphism [SNP]) profiling of CD40 and other genes involved in the CD40/CD40L axis, as certain CD40 SNPs have been identified as susceptibility loci in SLE or in certain disease subsets in Graves' disease. In aspects, blood DNA is collected for epigenetics analysis, such as DNA methylation, in immune related genes. In aspects, epigenome profiling is assessed by methods including, but not limited to, ATACseq and DNA methylation sequencing.

In aspects, expression levels of genes associated with disease activity by way of RNA and/or DNA analysis may be reduced by at least about 10%, 15%, 20%, 25%, 50%, 60%, 70%, 80%, 90%, 95%, 96%, 97%, 98%, 99%, or up to 100% as compared to an otherwise comparable method wherein the subject of the otherwise comparable method is not administered a Tn3 scaffold.

In aspects, a RNA and/or DNA analysis to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, a RNA and/or DNA analysis to a Tn3 scaffold can be assessed on about Day 1, Day 15±1, Day 29±3, Day 57±3, Day 85±3, Day 141±3, Day 169±3, Day 197±3, Day 225±3, Day 253±3, Day 281±3, Day 309±7, and Day 365±7 post treatment initiation. In aspects, an RNA and/or DNA assessment can occur at any point before, during, or after administration with a Tn3 scaffold.

### Actigraphy

In aspects, an assessment may comprise a measurement of overall activity and/or sleep patterns. In aspects, an assessment may comprise actigraphy. In aspects, an actigraphy is collected throughout the study to measure overall activity and sleep patterns, including but not limited to duration and/or fragmentation of sleep. In aspects, the activity monitor device is ActiGraph, a wearable watch device. In aspects, overall activity and changes in sleep patterns during the study may be determined.

In aspects, an actigraphy analysis to a Tn3 scaffold can be assessed on about 0 day, 1 day, 2 days, 3 days, 4 days, 5 days, 6 days, 1 week, 2 weeks, 3 weeks, 4 weeks, 5 weeks, 6 weeks, 7 weeks, 8 weeks, 9 weeks, 10 weeks, 11 weeks, 12 weeks, 13 weeks, 14 weeks, 15 weeks, 16 weeks, 17 weeks, 18 weeks, 19 weeks, 20 weeks, 21 weeks, 22 weeks, 23 weeks, 24 weeks, 25 weeks, 26 weeks, 27 weeks, 28 weeks, 29 weeks, 30 weeks, 31 weeks, 32 weeks, 33 weeks, 34 weeks, 35 weeks, 36 weeks, 37 weeks, 38 weeks, 39 weeks, 40 weeks, 41 weeks, 42 weeks, 43 weeks, 44 weeks, 45 weeks, 46 weeks 47 weeks, 48 weeks, 49 weeks, 50 weeks, 51 weeks, 52 weeks, or at least about 53 weeks post treatment initiation. In aspects, an actigraphy analysis to a Tn3 scaffold can be assessed on about Day 365±7 post treatment initiation. In aspects, an actigraphy assessment can occur at any point before, during, or after administration with a Tn3 scaffold.

### Response Duration (Duration of Clinical Response)

In aspects, an assessment comprises determining duration of a response to a Tn3 scaffold. In aspects, an assessment comprises determining duration of a response to a Tn3 scaffold by way of quantifying time to initiation of a rescue therapy. In aspects, administration of a Tn3 scaffold is effective in reducing or eliminating initiation of a rescue therapy in a treated subject as compared to a subject not administered a Tn3 scaffold. In aspects, administration of a Tn3 scaffold is effective at extending time to initiation of rescue therapy by at least about: 1 day, 6 days, 11 days, 16 days, 21 days, 26 days, 30 days, 2 months, 3 months, 4 months, 5 months, 6 months, 9 months, 11 months, 1 year, 2 years, or up to about 5 years post administration.

### Pharmaceutical Composition

In aspects, provided is a pharmaceutical composition. A pharmaceutical composition can comprise a Tn3 scaffold of the disclosure. In aspects, a pharmaceutical composition is part of a therapeutic regimen that comprises a Tn3 scaffold of the disclosure, and one or more additional therapeutics provided herein.

Many drugs can be administered orally as liquids, capsules, tablets, or chewable tablets. Because the oral route is the most convenient and usually the safest and least expensive, it is the one most often used. However, it has limitations because of the way a drug typically moves through the digestive tract. For drugs administered orally, absorption may begin in the mouth and stomach. However, most drugs are usually absorbed from the small intestine. The drug passes through the intestinal wall and travels to the liver before being transported via the bloodstream to its target site. The intestinal wall and liver chemically alter (metabolize) many drugs, decreasing the amount of drug reaching the bloodstream. Consequently, these drugs are often given in smaller doses when injected intravenously to produce the same effect.

For a subcutaneous route, a needle is inserted into fatty tissue just beneath the skin. After a drug is injected, it then moves into small blood vessels (capillaries) and is carried away by the bloodstream. Alternatively, a drug reaches the bloodstream through the lymphatic vessels. The intramuscular route is preferred to the subcutaneous route when larger volumes of a drug product are needed. Because the muscles lie below the skin and fatty tissues, a longer needle is used. Drugs are usually injected into the muscle of the upper arm, thigh, or buttock. How quickly the drug is absorbed into the bloodstream depends, in part, on the blood supply to the muscle: The sparser the blood supply, the longer it takes for the drug to be absorbed. For the intravenous route, a needle is inserted directly into a vein. A solution containing the drug may be given in a single dose or by continuous infusion. For infusion, the solution is moved by gravity (from a collapsible plastic bag) or, more commonly, by an infusion pump through thin flexible tubing to a tube (catheter) inserted in a vein, usually in the forearm.

In aspects, a pharmaceutical composition provided herein is administered via infusion. An infusion can take place over a period of time. For example, an infusion can be an administration of a pharmaceutical over a period of about 5 minutes to about 10 hours. An infusion can take place over a period of about 5 min, 10 min, 20 min, 30 min, 40 min, 50 min, 1 hour, 1.5 hours, 2 hours, 2.5 hours, 3 hours, 3.5 hours, 4 hours, 4.5 hours, 5 hours, 6 hours, 7 hours, 8 hours, 9 hours, or up to about 10 hours. In aspects, intravenous administration is used to deliver a precise dose quickly and in a well-controlled manner throughout the body. It is also used for irritating solutions, which would cause pain and damage tissues if given by subcutaneous or intramuscular injection. When given intravenously, a drug is delivered immediately to the bloodstream and tends to take effect more quickly than when given by any other route. Consequently, health care practitioners closely monitor people who receive an intravenous injection for signs that the drug is working or is causing undesired side effects. Also, the effect of a drug given by this route tends to last for a shorter time. Therefore, some drugs must be given by continuous infusion to keep their effect constant. In aspects, infusion reactions can occur and include headache, nausea, somnolence, dyspnea, fever, myalgia, rash, or other symptoms. Potential risks associated with administration of a Tn3 scaffold are infection, redness, swelling, pain, and induration at the administration site. Prior to each IV infusion subjects may receive prophylaxis with IV methylprednisolone, oral diphenhydramine, and oral acetaminophen, or equivalent(s) to reduce the risk or severity of potential reactions.

In aspects, a pharmaceutical is administered intrathecally. For the intrathecal route, a needle is inserted between two vertebrae in the lower spine and into the space around the spinal cord. The drug is then injected into the spinal canal. A small amount of local anesthetic is often used to numb the injection site. This route is used when a drug is needed to produce rapid or local effects on the brain, spinal cord, or the layers of tissue covering them (meninges)—for example, to treat infections of these structures.

Drugs administered by inhalation through the mouth can be atomized into smaller droplets than those administered by the nasal route, so that the drugs can pass through the windpipe (trachea) and into the lungs. How deeply into the lungs they go depends on the size of the droplets. Smaller droplets go deeper, which increases the amount of drug absorbed. Inside the lungs, they are absorbed into the bloodstream. Drugs applied to the skin are usually used for their local effects and thus are most commonly used to treat superficial skin disorders, such as psoriasis, eczema, skin infections (viral, bacterial, and fungal), itching, and dry skin. The drug is mixed with inactive substances. Depending on the consistency of the inactive substances, the formulation may be an ointment, cream, lotion, solution, powder, or gel.

In aspects, a treatment regime comprising a pharmaceutical composition may be dosed according to a body weight of a subject. In subjects who are determined obese (BMI>35) a practical weight may need to be utilized. BMI is calculated by BMI=weight (kg)/[height (m)]2. An ideal body weight may be calculated for men as 50 kg+2.3*(number of inches over 60 inches) or for women 45.5 kg+2.3 (number of inches over 60 inches). An adjusted body weight may be calculated for subjects who are more than 20% of their ideal body weight. An adjusted body weight may be the sum of an ideal body weight+(0.4×(Actual body weight−ideal body weight)). In aspects, body surface area may be utilized to calculate a dosage. A body surface area (BSA) may be calculated by: BSA (m2)=VHeight (cm)*Weight (kg)/3600.

In aspects, a pharmaceutical composition can be administered either alone or together with a pharmaceutically acceptable carrier or excipient, by any routes, and such administration can be carried out in both single and multiple dosages. More particularly, a pharmaceutical composition can be combined with various pharmaceutically acceptable inert carriers in the form of tablets, capsules, lozenges, troches, hand candies, powders, sprays, aqueous suspensions, injectable solutions, elixirs, syrups, and the like. Such carriers include solid diluents or fillers, sterile aqueous media and various non-toxic organic solvents, etc. Moreover, pharmaceutical formulations can be suitably sweetened and/or flavored by means of various agents of the type commonly employed for such purposes. Exemplary carriers and excipients can include dextrose, sodium chloride (NaCl), sucrose, lactose, cellulose, xylitol, sorbitol, maltitol, gelatin, PEG, PVP, histidine/histidine hydrochloride, trehalose dihydrate, polysorbate 80, and any combination thereof. In aspects, an excipient comprises: histidine/histidine hydrochloride, NaCl, trehalose dihydrate, and polysorbate 80.

### NUMBERED EMBODIMENTS

Notwithstanding the appended claims, the following numbered embodiments also form part of the instant disclosure.

### Embodiment Set 1

Embodiment 1. A method for treating Sjögren's syndrome (SS) in a subject in need thereof comprising: administering a Tn3 scaffold comprising a CD40L-specific monomer subunit to the subject; wherein the Tn3 scaffold binds to CD40L; wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, wherein the Tn3 scaffold comprising the CD40L-specific monomer subunit is administered at a dose of about 1500 mg, about 2500 mg, about 3000 mg, about 75-1500 mg, or about 300-3000 mg.

Embodiment 2. The method of embodiment 1, wherein at least one CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21.

Embodiment 3. The method of embodiments 1-2, wherein the subject has a European League Against Rheumatism (EULAR) Sjögren's Syndrome Disease Activity Index (ESSDAI) score≥5.

Embodiment 4. The method of embodiment 3, wherein the ESSDAI score is assessed based on the ESSDAI domains consisting of cutaneous, renal, articular, muscular, hematological, glandular, constitutional, lymphadenopathic, and biological.

Embodiment 5. The method of embodiments 1-2, wherein the subject has (a) said ESSDAI score≤5, (b) EULAR Sjögren's Syndrome Patient Reported Index (ESSPRI) score≥5, and (c) whole stimulated salivary flow>0.1 mL/min.

Embodiment 6. The method of any one of embodiments 1-5, wherein the Tn3 scaffold is administered as an induction dose and as a maintenance dose thereafter.

Embodiment 7. The method of embodiment 6, wherein the induction dose comprises administering the Tn3 scaffold once about every 2 weeks for at least 3 doses.

Embodiment 8. The method of embodiment 6, wherein the maintenance dose comprises administering the Tn3 scaffold once about every 4 weeks for at least 4 doses.

Embodiment 9. The method of embodiment 8, wherein the time between the last induction dose and the first maintenance dose is about 4 weeks.

Embodiment 10. The method of any one of embodiments 1-5, wherein the Tn3 scaffold is administered once about every 4 weeks, once about every 2 months, once about every 3 months, once about every 4 months, or once about every 6 months.

Embodiment 11. The method of embodiment 10, wherein the Tn3 scaffold is administered for at least 4 doses.

Embodiment 12. The method of embodiment 11, wherein the Tn3 scaffold is administered for at least 5 doses.

Embodiment 13. The method of any one of embodiments 1-12, wherein the Tn3 scaffold is administered intravenously, subcutaneously, orally, intramuscularly, intrathecally, sublingually, rectally, vaginally, cutaneously, systemically, topically, transdermally, or by way of inhalation.

Embodiment 14. The method of claim 13, wherein the Tn3 scaffold is administered intravenously.

Embodiment 15. The method of any one of embodiments 1-13, wherein the Tn3 scaffold comprises two CD40L-specific monomer subunits connected in tandem.

Embodiment 16. The method of embodiment 15 wherein the two CD40L-specific monomer subunits each comprise SEQ ID NO: 3.

Embodiment 17. The method of any of one of embodiments 1-16, wherein the CD40L-specific monomer subunits are connected by a linker.

Embodiment 18. The method of any one of embodiments 1-17, wherein at least one CD40L-specific monomer subunit is fused or conjugated to a polyethylene glycol (PEG) directly.

Embodiment 19. The method of any one of embodiments 1-17 wherein at least one CD40L-specific monomer subunit is fused or conjugated to a polyethylene glycol (PEG) via a linker.

Embodiment 20. The method of embodiment 17, wherein the linker comprises a peptide linker.

Embodiment 21. The method of embodiment 20, wherein the linker comprises SEQ ID NO: 7, SEQ ID NO: 8, SEQ ID NO: 9, or SEQ ID NO: 10.

Embodiment 22. The method of embodiment 21, wherein at least one CD40L-specific monomer subunit is fused or conjugated to an albumin.

Embodiment 23. The method of embodiment 22, wherein the albumin is human serum albumin (HSA).

Embodiment 24. The method of embodiment 23, wherein the HSA is a variant HSA comprising SEQ ID NO: 4.

Embodiment 25. A method of treating Sjögren's syndrome (SS) in a subject in need thereof comprising: administering a Tn3 scaffold at a dose of about 1500 mg, about 2500 mg, or about 3000 mg to the subject, wherein the Tn3 scaffold comprises SEQ ID NO: 1.

Embodiment 26. The method of embodiment 25 wherein the Tn3 scaffold is administered as an induction dose and as one or more maintenance doses thereafter.

Embodiment 27. The method of embodiment 26 wherein the induction dose and the maintenance doses are the same amount.

Embodiment 28. The method of embodiment 27 wherein the induction dose and the maintenance doses are different amounts.

Embodiment 29. The method of any one of embodiments 25-28 wherein at least one dose is 3000 mg.

Embodiment 30. The method of any one of embodiments 25-29 wherein the induction dose and at least one maintenance dose are administered about 1 month apart, about 2 months apart, about 3 months apart, about 4 months, or about 6 months apart.

Embodiment 31. The method of any one of embodiments 25-30, wherein the induction dose comprises administering the Tn3 scaffold about every 2 weeks for at least 3 doses and the maintenance dose comprises administering the Tn3 scaffold once about every 4 weeks for at least 4 doses.

Embodiment 32. The method of any one of embodiments 1-31, wherein the Tn3 scaffold comprises SEQ ID NO: 1.

Embodiment 33. The method of any one of embodiments 1-32, wherein the administration is effective in reducing a ESSDAI score as compared to an otherwise comparable subject receiving an administration of placebo control.

Embodiment 34. The method of embodiment 33, wherein the reduction is of at least about 1 point, 2 points, 3 points, 4 points, or 5 points.

Embodiment 35. The method of embodiment 33, wherein the reduction is of at least about 6 points.

### Embodiment Set 2

1. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the subject in need has increased B cells of subsets selected from the group consisting of: Ki67+CD27+ memory cells, CD27 high CD38 high plasmablasts, and CD11c high atypical memory cells, as compared to a baseline level of the subject or a level in an untreated subject.

2. The method of embodiment 1, wherein at least one CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21.

3. The method of embodiments 1-2, wherein the subject has a European League Against Rheumatism (EULAR) Sjögren's Syndrome Disease Activity Index (ESSDAI) score≥5.

4. The method of embodiment 3, wherein the ESSDAI score is assessed based on the ESSDAI domains consisting of cutaneous, renal, articular, muscular, hematological, glandular, constitutional, lymphadenopathic, and biological.

5. The method of embodiments 1-2, wherein the subject has (a) a ESSDAI score≤5, (b) a EULAR Sjögren's Syndrome Patient Reported Index (ESSPRI) score≥5, and (c) whole stimulated salivary flow>0.1 mL/min.

6. The method of any one of embodiments 1-5, wherein the Tn3 scaffold is administered as an induction dose and as a maintenance dose thereafter.

7. The method of embodiment 6, wherein the induction dose comprises administering the Tn3 scaffold once about every 2 weeks for at least 3 doses.

8. The method of embodiment 6, wherein the maintenance dose comprises administering the Tn3 scaffold once about every 4 weeks for at least 4 doses.

9. The method of embodiment 8, wherein the time between the last induction dose and the first maintenance dose is about 4 weeks.

10. The method of any one of embodiments 1-5, wherein the Tn3 scaffold is administered once about every 4 weeks, once about every 2 months, once about every 3 months, once about every 4 months, or once about every 6 months.

11. The method of embodiment 10, wherein the Tn3 scaffold is administered for at least 4 doses.

12. The method of embodiment 11, wherein the Tn3 scaffold is administered for at least 5 doses.

13. The method of any one of embodiments 1-12, wherein the Tn3 scaffold is administered intravenously, subcutaneously, orally, intramuscularly, intrathecally, sublingually, rectally, vaginally, cutaneously, systemically, topically, transdermally, or by way of inhalation.

14. The method of embodiment 13, wherein the Tn3 scaffold is administered intravenously.

15. The method of any one of embodiments 1-14, wherein the Tn3 scaffold comprises two CD40L-specific monomer subunits connected in tandem.

16. The method of embodiment 15, wherein the two CD40L-specific monomer subunits each comprise SEQ ID NO: 3.

17. The method of any of one of embodiments 1-16, wherein the CD40L-specific monomer subunits are connected by a linker.

18. The method of any one of embodiments 1-17, wherein at least one CD40L-specific monomer subunit is fused or conjugated to a polyethylene glycol (PEG) directly.

19. The method of any one of embodiments 1-17 wherein at least one CD40L-specific monomer subunit is fused or conjugated to a polyethylene glycol (PEG) via a linker.

20. The method of embodiment 17, wherein the linker comprises a peptide linker.

21. The method of embodiment 20, wherein the linker comprises SEQ ID NO: 7, SEQ ID NO: 8, SEQ ID NO: 9, or SEQ ID NO: 10.

22. The method of embodiment 21, wherein at least one CD40L-specific monomer subunit is fused or conjugated to an albumin.

23. The method of embodiment 22, wherein the albumin is human serum albumin (HSA).

24. The method of embodiment 23, wherein the HSA is a variant HSA comprising SEQ ID NO: 4.

25. The method of any one of embodiments 1-24, wherein the Tn3 scaffold comprising the CD40L-specific monomer subunit is administered at a dose of about 1500 mg, about 2500 mg, or about 3000 mg.

26. The method of embodiment 25, wherein the dose is about 1500 mg.

27. The method of embodiment 25, wherein the dose is about 2500 mg.

28. The method of embodiment 25, wherein the dose is about 3000 mg.

29. A method of treating Sjögren's syndrome (SS) in a subject in need thereof comprising: administering a Tn3 scaffold at a dose of about 1500 mg, about 2500 mg, or about 3000 mg to the subject, wherein the Tn3 scaffold comprises SEQ ID NO: 1, and wherein the subject in need has increased B cells of subsets selected from the group consisting of: Ki67+CD27+ memory cells, CD27 high CD38 high plasmablasts, and CD11c high atypical memory cells, as compared to an otherwise healthy subject.

30. The method of embodiment 29, wherein the Tn3 scaffold is administered as an induction dose and as one or more maintenance doses thereafter.

31. The method of embodiment 30, wherein the induction dose and the maintenance doses are the same amount.

32. The method of embodiment 30, wherein the induction dose and the maintenance doses are different amounts.

33. The method of any one of embodiments 29-32, wherein at least one dose is 3000 mg.

34. The method of any one of embodiments 29-33, wherein the induction dose and at least one maintenance dose are administered about 1 month apart, about 2 months apart, about 3 months apart, about 4 months, or about 6 months apart.

35. The method of any one of embodiments 29-34, wherein the induction dose comprises administering the Tn3 scaffold about every 2 weeks for at least 3 doses and the maintenance dose comprises administering the Tn3 scaffold once about every 4 weeks for at least 4 doses.

36. The method of any one of embodiments 1-35, wherein the Tn3 scaffold comprises SEQ ID NO: 1.

37. The method of any one of embodiments 1-36, wherein the administration is effective in reducing a ESSDAI score as compared to an otherwise comparable subject receiving an administration of placebo control.

38. The method of embodiment 37, wherein the reduction is of at least about 1 point, 2 points, 3 points, 4 points, or 5 points.

39. The method of embodiment 38, wherein the reduction is of at least about 6 points.

40. The method of any one of embodiments 1-39, wherein the administration is effective in reducing levels of B cells as compared to the baseline level of the subject or the level in the untreated subject.

41. The method of any one of embodiments 1-40, wherein the administration is effective in reducing a level of serum CXCL13 as compared to a baseline level in the subject or a level in an untreated subject.

42. The method of any one of embodiments 1-41, wherein the administration is effective in reducing rheumatoid factor (RF) autoantibodies as compared to a baseline level in the subject or a level in an untreated subject.

43. The method of any one of embodiments 40-42, wherein the reducing is detected at least by day 14 following the administering.

44. A method for treating autoimmune disease in a subject in need thereof comprising administering a Tn3 scaffold comprising a CD40L-specific monomer subunit that binds CD40L to the subject when a level of a biomarker is increased in a sample of the subject as compared to a healthy baseline level, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16.

45. The method of embodiment 44, wherein the biomarker is expressed on the surface of a cell.

46. The method of embodiment 44, wherein the biomarker is an antibody.

47. The method of embodiment 44, wherein the biomarker is secreted by a cell.

48. The method of any one of embodiments 44-47, wherein the biomarker is selected from the group consisting of: Ki67+CD27+ memory cell, CD27 high CD38 high plasmablast, CD11c high atypical memory cell, CXCL13, RF autoantibody, and any combination thereof.

49. The method of any one of embodiments 44-48, wherein the autoimmune disease is selected from the group consisting of: systemic lupus erythematosus (SLE), lupus nephritis, cutaneous lupus erythematosus (CLE), Sjögren's syndrome, dermatomyositis, myositis, sclerosis, diabetes, Hashimoto's disease, autoimmune adrenal insufficiency, anemia, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy.

50. The method of any one of embodiments 44-49, wherein the administering is continued until the level of the biomarker reverts to the healthy baseline.

51. The method of any one of embodiments 44-50, wherein the level of the biomarker is analyzed routinely.

52. The method of any one of embodiments 44-51, wherein the Tn3 scaffold comprises SEQ ID NO: 1.

53. The method of any one of embodiments 44-52, wherein the Tn3 scaffold is administered at a dose of about 1500 mg, about 2500 mg, or about 3000 mg.

54. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the treatment of the subject with the Tn3 scaffold decreases the level of B cells selected from the group consisting of: Ki67+CD27+ memory cells, CD27 high CD38 high plasmablasts, and CD11c high atypical memory cells, as compared to a baseline level of the subject or a level in an untreated subject.

55. The method of embodiment 54, wherein the Tn3 scaffold comprises SEQ ID NO: 1.

56. The method of any one of embodiments 54-55, wherein the Tn3 scaffold is administered at a dose of about 1500 mg, about 2500 mg, or about 3000 mg.

57. The method of any one of embodiments 54-56, wherein the level of B cells is reduced by at least about 1-fold, 5-fold, 10-fold, 30-fold, 50-fold, 75-fold, 100-fold, 150-fold, or 200-fold.

### Embodiment Set 3

1. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the subject in need has increased B cells of subsets selected from the group consisting of: Ki67+CD27+ memory cells, CD27 high CD38 high plasmablasts, and CD11c high atypical memory cells, as compared to a baseline level of the subject or a level in an untreated subject.

2. The method of embodiment 1, wherein at least one CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21.

3. The method of embodiments 1-2, wherein the subject has a European League Against Rheumatism (EULAR) Sjögren's Syndrome Disease Activity Index (ESSDAI) score≥5.

4. The method of embodiment 3, wherein the ESSDAI score is assessed based on the ESSDAI domains consisting of cutaneous, renal, articular, muscular, hematological, glandular, constitutional, lymphadenopathic, and biological.

5. The method of embodiments 1-2, wherein the subject has
(a) a ESSDAI score<5,
(b) a EULAR Sjögren's Syndrome Patient Reported Index (ESSPRI) score≥5, and
(c) whole stimulated salivary flow>0.1 mL/min.

6. The method of any one of embodiments 1-5, wherein the Tn3 scaffold is administered as an induction dose and as a maintenance dose thereafter.

7. The method of embodiment 6, wherein the induction dose comprises administering the Tn3 scaffold once about every 2 weeks for at least 3 doses.

8. The method of embodiment 6, wherein the maintenance dose comprises administering the Tn3 scaffold once about every 4 weeks for at least 4 doses.

9. The method of embodiment 8, wherein the time between the last induction dose and the first maintenance dose is about 4 weeks.

10. The method of any one of embodiments 1-5, wherein the Tn3 scaffold is administered once about every 4 weeks, once about every 2 months, once about every 3 months, once about every 4 months, or once about every 6 months.

11. The method of embodiment 10, wherein the Tn3 scaffold is administered for at least 4 doses.

12. The method of embodiment 11, wherein the Tn3 scaffold is administered for at least 5 doses.

13. The method of any one of embodiments 1-12, wherein the Tn3 scaffold is administered intravenously, subcutaneously, orally, intramuscularly, intrathecally, sublingually, rectally, vaginally, cutaneously, systemically, topically, transdermally, or by way of inhalation.

14. The method of embodiment 13, wherein the Tn3 scaffold is administered intravenously.

15. The method of any one of embodiments 1-14, wherein the Tn3 scaffold comprises two CD40L-specific monomer subunits connected in tandem.

16. The method of embodiment 15, wherein the two CD40L-specific monomer subunits each comprise SEQ ID NO: 3.

17. The method of any of one of embodiments 1-16, wherein the CD40L-specific monomer subunits are connected by a linker.

18. The method of any one of embodiments 1-17, wherein at least one CD40L-specific monomer subunit is fused or conjugated to a polyethylene glycol (PEG) directly.

19. The method of any one of embodiments 1-17 wherein at least one CD40L-specific monomer subunit is fused or conjugated to a polyethylene glycol (PEG) via a linker.

20. The method of embodiment 17, wherein the linker comprises a peptide linker.

21. The method of embodiment 20, wherein the linker comprises SEQ ID NO: 7, SEQ ID NO: 8, SEQ ID NO: 9, or SEQ ID NO: 10.

22. The method of embodiment 21, wherein at least one CD40L-specific monomer subunit is fused or conjugated to an albumin.

23. The method of embodiment 22, wherein the albumin is human serum albumin (HSA).

24. The method of embodiment 23, wherein the HSA is a variant HSA comprising SEQ ID NO: 4.

25. The method of any one of embodiments 1-24, wherein the Tn3 scaffold comprising the CD40L-specific monomer subunit is administered at a dose of about 1500 mg, about 2500 mg, or about 3000 mg.

26. The method of embodiment 25, wherein the dose is about 1500 mg.

27. The method of embodiment 25, wherein the dose is about 2500 mg.

28. The method of embodiment 25, wherein the dose is about 3000 mg.

29. A method of treating Sjögren's syndrome (SS) in a subject in need thereof comprising:

administering a Tn3 scaffold at a dose of about 1500 mg, about 2500 mg, or about 3000 mg to the subject, wherein the Tn3 scaffold comprises SEQ ID NO: 1, and wherein the subject in need has increased B cells of subsets selected from the group consisting of: Ki67+CD27+ memory cells, CD27 high CD38 high plasmablasts, and CD11c high atypical memory cells, as compared to an otherwise healthy subject.

30. The method of embodiment 29, wherein the Tn3 scaffold is administered as an induction dose and as one or more maintenance doses thereafter.

31. The method of embodiment 30, wherein the induction dose and the maintenance doses are the same amount.

32. The method of embodiment 30, wherein the induction dose and the maintenance doses are different amounts.

33. The method of any one of embodiments 29-32, wherein at least one dose is 3000 mg.

34. The method of any one of embodiments 29-33, wherein the induction dose and at least one maintenance dose are administered about 1 month apart, about 2 months apart, about 3 months apart, about 4 months, or about 6 months apart.

35. The method of any one of embodiments 29-34, wherein the induction dose comprises administering the Tn3 scaffold about every 2 weeks for at least 3 doses and the maintenance dose comprises administering the Tn3 scaffold once about every 4 weeks for at least 4 doses.

36. The method of any one of embodiments 1-35, wherein the Tn3 scaffold comprises SEQ ID NO: 1.

37. The method of any one of embodiments 1-36, wherein the administration is effective in reducing a ESSDAI score as compared to an otherwise comparable subject receiving an administration of placebo control.

38. The method of embodiment 37, wherein the reduction is of at least about 1 point, 2 points, 3 points, 4 points, or 5 points.

39. The method of embodiment 38, wherein the reduction is of at least about 6 points.

40. The method of any one of embodiments 1-39, wherein the administration is effective in reducing levels of B cells as compared to the baseline level of the subject or the level in the untreated subject.

41. The method of any one of embodiments 1-40, wherein the administration is effective in reducing a level of serum CXCL13 as compared to a baseline level in the subject or a level in an untreated subject.

42. The method of any one of embodiments 1-41, wherein the administration is effective in reducing rheumatoid factor (RF) autoantibodies as compared to a baseline level in the subject or a level in an untreated subject.

43. The method of any one of embodiments 40-42, wherein the reducing is detected at least by day 14 following the administering.

44. A method for treating autoimmune disease in a subject in need thereof comprising administering a Tn3 scaffold comprising a CD40L-specific monomer subunit that binds CD40L to the subject when a level of a biomarker is increased in a sample of the subject as compared to a healthy baseline level, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16.

45. The method of embodiment 44, wherein the biomarker is expressed on the surface of a cell.

46. The method of embodiment 44, wherein the biomarker is an antibody.

47. The method of embodiment 44, wherein the biomarker is secreted by a cell.

48. The method of any one of embodiments 44-47, wherein the biomarker is selected from the group consisting of: Ki67+CD27+ memory cell, CD27 high CD38 high plasmablast, CD11c high atypical memory cell, CXCL13, RF autoantibody, and any combination thereof.

49. The method of any one of embodiments 44-48, wherein the biomarker is selected from the group consisting of TNFRSF13B, BAFF, CXCL10, CXCL12, IL6, CCL21, CXCL9, IL12B, TNFRSF4, LAMP3, CD22, CD79B, SLAMF7, IL10, TNFRSF8, CXCL11, CCL19, TNFRSF9, TNFRSF6B, NOS3, CD72, ADA2, IL2RA, CD27, PDCD1, IL12A, IL12B, LAG3, ADGRE1, TIGIT, FCAMR, and CD80, and any combination thereof.

50. The method of any one of embodiments 44-49, wherein the autoimmune disease is selected from the group consisting of: systemic lupus erythematosus (SLE), lupus nephritis, cutaneous lupus erythematosus (CLE), Sjögren's syndrome, dermatomyositis, myositis, sclerosis, diabetes, Hashimoto's disease, autoimmune adrenal insufficiency, anemia, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy.

51. The method of embodiment 50, wherein the arthritis is rheumatoid arthritis.

52. The method of any one of embodiments 44-51, wherein the administering is continued until the level of the biomarker reverts to the healthy baseline.

53. The method of any one of embodiments 44-52, wherein the level of the biomarker is analyzed routinely.

54. The method of any one of embodiments 44-53, wherein the Tn3 scaffold comprises SEQ ID NO: 1.

55. The method of any one of embodiments 44-54, wherein the Tn3 scaffold is administered at a dose of about 1500 mg, about 2500 mg, or about 3000 mg.

56. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the treatment of the subject with the Tn3 scaffold decreases the level of B cells selected from the group consisting of: Ki67+CD27+ memory cells, CD27 high CD38 high plasmablasts, and CD11c high atypical memory cells, as compared to a baseline level of the subject or a level in an untreated subject.

57. The method of embodiment 56, wherein the Tn3 scaffold comprises SEQ ID NO: 1.

58. The method of any one of embodiments 56-57, wherein the Tn3 scaffold is administered at a dose of about 1500 mg, about 2500 mg, or about 3000 mg.

59. The method of any one of embodiments 56-58, wherein the level of B cells is reduced by at least about 1-fold, 5-fold, 10-fold, 30-fold, 50-fold, 75-fold, 100-fold, 150-fold, or 200-fold.

60. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the Tn3 scaffold is administered to a subject having increased levels of T cells selected from a subset of the group consisting of: Ki67+/CD3+, Ki67+/CD4+ cells, Ki67+/CD3+/CD4+ cells, Ki67+/CD3+/CD4+/CD45RA−/CCRT7+ cells, Ki67+/CD3+/CD4+/CD45RA−/CCR7−cells, Ki67+/CD3+/CD4+/CXCR5+/ICOS+ cells, Ki67+/CD3+/CD8+ cells, Ki67+/CD3+/CD8+/CD45RA−/CCR7+ cells, Ki67+/CD3+/CD8+/CD45RA/CCR7−cells, and Ki67+/CD3+/CD8+/CXCR5+ cells, and CXCR5+/ICOS+/CD3+/CD4+ cells, as compared to a baseline level of the subject or a level in an untreated subject.

61. The method of embodiment 60, wherein the Tn3 scaffold comprises SEQ ID NO: 1.

62. The method of any one of embodiments 60-61, wherein the Tn3 scaffold is administered at a dose of about 1500 mg, about 2500 mg, or about 3000 mg.

63. The method of any one of embodiments 60-62, wherein the level of T cells is reduced by at least about 1-fold, 5-fold, 10-fold, 30-fold, 50-fold, 75-fold, 100-fold, 150-fold, or 200-fold.

### Examples

### Example 1—a Phase 2 Randomized, Double-Blind, Placebo-Controlled, Proof of Concept Study to Evaluate the Efficacy and Safety of VIB4920 in Subjects with Sjögren's Syndrome (SS)

### Study Design

The study was a randomized, double-blind, placebo-controlled, parallel-arm study to evaluate the efficacy, safety, and tolerability of VIB4920, an anti-CD40L-Tn3 fusion protein in adult subjects with SS diagnosed according to the 2016 American College of Rheumatology (ACR)/European League Against Rheumatism (EULAR) Criteria. The study enrolled 2 SS populations. Population #1 was composed of subjects with moderate to severe (moderate to high) systemic disease activity defined by ESSDAI≥5. Population #2 was composed of subjects with mild systemic disease activity defined by ESSDAI score<5 but with moderate to high (or severe) subjective symptoms defined by ESSPRI score≥5 and residual stimulated salivary flow.

Within each population, eligible subjects were randomized to receive VIB4920 1500 mg IV or placebo once every two weeks (Q2W)×3 doses, then once every 4 weeks (Q4W) for 4 additional doses (Stage I). Starting on Day 169, subjects randomized to VIB4920 received placebo Q4W for 5 doses and subjects randomized to placebo received VIB4920 Q4W for 5 doses (Stage II). Subjects who had VIB4920 discontinuation were not eligible for treatment during Stage II. All subjects were followed for at least 12 weeks after their last dose of IP administration.

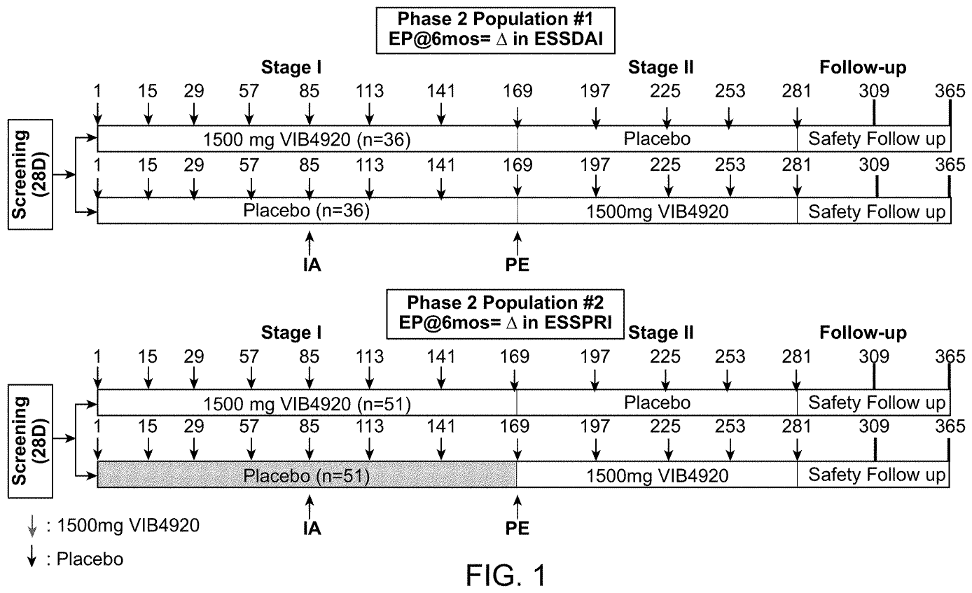

An exemplary study schematic is presented in FIG. 1.

### Population

The 2 SS populations distinguish patients with predominantly glandular dysfunction and those with both glandular dysfunction and systemic manifestations. The 2 SS populations represent subsets with significant unmet need that require different endpoints to assess efficacy. The ESSDAI is a validated instrument to assess systemic disease activity. The ESSPRI uses a 0-10 numerical analog scale, one for the assessment of each of the 3 domains of SS symptoms: dryness, fatigue, and pain (articular and/or muscular).

Population #1 comprised SS patients with moderate to high systemic activity, defined by ESSDAI≥5. The following domains were scored but they did not contribute to the minimum ESSDAI score of 5 required for inclusion: peripheral nervous system, central nervous system, and pulmonary. Population #2 comprised SS patients with exocrine dysfunction and subjective symptoms defined by an ESSPRI score of >5, considered as the cut-off point for “unsatisfactory symptom state” but lower systemic disease activity but with lower systemic disease activity (with ESSDAI score≤5).

A complete physical examination, including weight, height, and 28-joint assessment was conducted. The 2016 ACR/EULAR Criteria was used for classification of SS for both population #1 and population #2, as confirmed by medically qualified individuals. The classification of SS applied to any individual who met the inclusion criteria,¹ did not have any condition listed as exclusion criteria,² and who had a score≥4 when summing the weights from the following items shown in Table 2.

TABLE 2
ACR-EULAR classification criteria for
primary Sjögren's syndrome

| Item | Weight / Score |
|---|---|
| Labial salivary gland with focal lymphocytic | 3 |
| sialadenitis and focus score ≥1.³ | |
| Anti-SSA (Ro)+ | 3 |
| Ocular staining score ≥5 (or van Bijsterveld score ≥4) | 1 |
| on at least one eye⁴ | |
| Schirmer ≤5 mm/5 min on at least one eye | 1 |
| Unstimulated whole saliva flow rate ≤0.1 ml/min⁵ | 1 |

1 Inclusion criteria: these criteria are applicable to any subject with at least one symptom of ocular or oral dryness (defined as a positive response to at least one of the following questions: 1) Have you had daily, persistent, troublesome dry eyes for more than 3 months? 2) Do you have a recurrent sensation of sand or gravel in the eyes? 3) Do you use tear substitutes more than 3 times a day? 4) Have you had a daily feeling of dry mouth for more than 3 months? 5) Do you frequently drink liquids to aid in swallowing dry food?); or suspicion of SS from ESSDAI questionnaire (at least one domain with positive item)
2 Exclusion criteria: Prior diagnosis of any of the following conditions would exclude diagnosis of SS and participation in SS studies or therapeutic trials because of overlapping clinical features or interference with criteria tests:
History of head and neck radiation treatment
Active Hepatitis C infection (with positive PCR)
Acquired immunodeficiency syndrome
Sarcoidosis
Amyloidosis
Graft versus host disease
IgG4-related disease
Note:
Patients who are normally taking anticholinergic drugs should be evaluated for objective signs of salivary hypofunction and ocular dryness after a sufficient interval off these medications for these components to be a valid measure of oral and ocular dryness
³The histopathologic examination should be performed by a pathologist with expertise in the diagnosis of focal lymphocytic sialadenitis, and focus score count (based on number of foci per 4 mm²) following a protocol described in Daniels et al Arthritis Rheum. 2011 July; 63(7): 2021-30.
⁴Ocular staining score described in Whitcher et al., Am J Ophthalmol. 2009; 149(3): 405-15; van Bijsterfeld score (described in van Bijsterfeld Arch Ophthalmol. 1969 July; 82(1): 10-4)
⁵Unstimulated whole saliva described Navazesh and Kumar (J Am Dent Assoc. 2008 May; 139 Suppl: 35S-40S).

Eligibility related to ESSDAI scores for Population #1 was also confirmed by medically qualified individuals. Whole stimulated salivary flow, ESSDAI, and ESSPRI was conducted at screening to determine study eligibility. ESSDAI-required blood tests were also completed. Additionally, an autoantibody panel, which includes RF was collected.

### Inclusion Criteria for Population #1

To be included in Population #1 of this study, each individual may have satisfied the following criteria:
1. Adults, 18 years or older at time of informed consent (the minimum age for adult participants can be higher than 18 years in countries with different regulations).
2. Diagnosed with SS by meeting the 2016 ACR/EULAR Classification Criteria.
3. Have an ESSDAI score of ≥5 at screening; the following domains were scored but they did not contribute to the minimum ESSDAI score of 5 required for inclusion: Peripheral nervous system, central nervous system, and pulmonary.
4. Positive for either anti-Ro autoantibodies or RF, or both at screening, as per the definition of the standard central laboratory test.
5. Written informed consent and any locally required authorization (e.g., Health Insurance Portability and Accountability Act in the United States, European Union Data Privacy Directive in the EU) obtained from the subject/legal representative prior to performing any protocol-related procedures, including screening evaluations.
6. Females of childbearing potential who are sexually active with a nonsterilized male partner must use a highly effective method of contraception from signing the informed consent form (ICF) and must agree to continue using such precautions through the end of the study follow-up; cessation of contraception after this point should be discussed with a responsible physician. Highly effective methods of contraception include:
   combined (estrogen and progestogen containing) hormonal contraception associated with inhibition of ovulation:
      oral
      intravaginal
      transdermal
   progestogen-only hormonal contraception associated with inhibition of ovulation:
      oral
      injectable
      implantable
   intrauterine device (IUD)
   intrauterine hormone-releasing system (IUS)
   bilateral tubal occlusion
   vasectomized partner
   sexual abstinence
Sexual abstinence is considered a highly effective method only if it is the preferred and usual lifestyle of the subject and the subject agrees to refrain from heterosexual intercourse from signing the ICF through the end of the study follow-up. Periodic abstinence, the rhythm method, and the withdrawal method are not acceptable methods of contraception. A recommendation that the female partners (of childbearing potential) of male study participants should use a highly effective method of contraception other than a barrier method is made.
   Females of childbearing potential are defined as those who are not surgically sterile (surgical sterilization includes bilateral tubal ligation, bilateral oophorectomy, or hysterectomy) or those who are not postmenopausal (defined as 12 months with no menses without an alternative medical cause).
   Vasectomized partner is a highly effective birth control method provided that partner is the sole sexual partner of the woman of childbearing potential trial participant and that the vasectomized partner has received medical assessment of the surgical success.
7. Nonsterilized male subjects who are sexually active with a female partner of childbearing potential must use a condom with spermicide from Day 1 through the end of the study.
8. Meets all of the following tuberculosis (TB) criteria:
   a. No history of latent or active TB prior to screening, with the exception of latent TB with documented completion of appropriate treatment.
   b. No signs or symptoms suggestive of active TB from medical history or physical examination.
   c. No recent (≤12 weeks of screening) close contact with a person with active TB (close contact is defined as ≥4 hours/week OR living in the same household OR in a house where a person with active TB is a frequent visitor).
   d. Negative Interferon Gamma Release Assay (IGRA) test result for TB obtained within 12 weeks prior to randomization. Subjects with an indeterminate test result can repeat the test, but if the repeat test is also indeterminate, they are excluded.
   e. A chest radiograph (obtained during the screening period or any time within 12 weeks prior to signing of the ICF) with no evidence of current active TB or other infection, or old active TB, malignancy, or clinically significant abnormalities suggesting an active process (unless due to SS).

If an individual for Population #1 met any of the following criteria, he or she was ineligible:
1. Patients with medical history of confirmed deep venous thrombosis or arterial thromboembolism within 2 years of signing the ICF.
2. Patients with risk factors for venous thromboembolism or arterial thrombosis (e.g., immobilization or major surgery within 12 weeks before screening), prothrombotic status (including, but not limited to, congenital or inherited deficiency of antithrombin III, protein C, protein S, or confirmed diagnosis of catastrophic antiphospholipid syndrome).
3. Patients requiring treatment with anticoagulant drugs (clopidogrel, prasugrel, warfarin, low molecular weight heparin, others). Low-dose aspirin treatment (up to 325 mg/day) is allowed.
4. Concomitant polymyositis or dermatomyositis or systemic sclerosis.
5. Active malignancy or history of malignancy within the last 5 years, except as follows:
   a. In situ carcinoma of the cervix treated with apparent success with curative therapy >12 months prior to screening; or
   b. Cutaneous basal cell carcinoma following apparently curative therapy.
6. Subjects who are pregnant or lactating or planning to become pregnant during the duration of the study.
7. Subjects who have a positive test for, or have been treated for hepatitis B, hepatitis C, or HIV infection.
   Regarding hepatitis B, positive test for chronic hepatitis B infection at screening, defined as either (1) positive hepatitis B surface antigen (HBsAg) or (2) a positive hepatitis B core antibody (anti-HBc).
8. Subjects with:
   a. A history of more than one episode of herpes zoster and/or opportunistic infections in the last 12 months, with the exception of oral candidiasis, vaginal candidiasis, and cutaneous fungal infections.
   b. Active viral, bacterial or other infections requiring systemic treatment at the time of screening or through randomization, or history of more than 2 infections requiring IV antibiotics within 12 months prior to signing the ICF.
   c. Epidemiologic risk of COVID-19 (recent exposures, high-risk housing) and for health-related risk of COVID-19 severity based on current understanding of risk factors for severe disease when making a decision regarding the individual subject's risk of participation. Subjects who have active COVID-19 infection or disease or other significant infection, or, in the judgment of the investigator, who may be at unacceptable risk of COVID-19, or its complications should not be randomized.
   d. A documented positive SARS-CoV-2 test within 2 weeks prior to randomization. Subjects with a positive test for SARS-CoV-2 may be rescreened at least 2 weeks after a positive test if asymptomatic and at least 3 weeks after symptomatic COVID-19 illness.
9. Subjects with known history of severe allergy or reaction to any component of VIB4920 formulation or to any other biologic therapy.
10. Subjects with any severe cardiovascular, respiratory, endocrine, gastrointestinal, hematological, neurological, psychiatric, or systemic disorder or any other condition that in the opinion of the Investigator, would place the subject at unacceptable risk of complications, interfere with evaluation of VIB4920 or confound the interpretation of subject safety or study results.
11. Subjects who are unable or unwilling to comply with protocol requirements (e.g., active drug or alcohol abuse).
12. Subjects who have received live (attenuated) vaccine within the 4 weeks prior to ICF signature.
13. Last administration of experimental biologic (other than those listed in Point 14) or oral agents<3 months or 5 half-lives before randomization.
14. Subjects who have had previous treatment with any biologic B-cell-depleting therapy (e.g., rituximab, ocrelizumab, or ofatumumab) within 12 months or other B-cell targeting therapy (e.g., belimumab)<3 months before randomization.
15. Injectable corticosteroids (including intra-articular) or treatment with >10 mg/day dose oral prednisone or equivalent within 6 weeks prior to randomization. Concomitant treatment with oral corticosteroids≤10 mg/day prednisone or equivalent is permitted provided that the dose is stable≥2 weeks prior to screening through randomization (Day 1) and is expected to remain stable for the duration of the treatment period. Inhaled or topical corticosteroids given for asthma, chronic obstructive pulmonary disease or dermatological conditions are allowed provided doses are expected to be stable during the study.
16. Subjects treated with systemic corticosteroids for indications other than SS, RA, and SLE for more than a total of 2 weeks within 24 weeks prior to screening visit.
17. Use of the following medications:
   a. Antimalarials (e.g., chloroquine, hydroxychloroquine, quinacrine) if they have been initiated or if the dose has changed within 8 weeks prior to signing the ICF or during the screening period.
   b. MTX, if the dose is >20 mg/week; or if there is any change or initiation of new dose within 4 weeks prior to signing the ICF through randomization (Day 1), or if there has been any change in route of administration.
   c. Azathioprine (AZA), if the dose is >150 mg/day and there is any change or initiation of new dose within 4 weeks prior to signing the ICF through randomization (Day 1) and any change in route of administration.
   d. Leflunomide, if the dose is >20 mg/day; or if there is any change or initiation of new dose within 4 weeks prior to signing the ICF through randomization (Day 1).
   e. Mycophenolate mofetil (MMF), if the dose is >2 g/day; or if there is any change or initiation of new dose within 4 weeks prior to signing the ICF through randomization (Day 1).
   f. Any other DMARD, immunosuppressant, or antiproliferative agents, if last dose was taken within:
      4 weeks prior to signing ICF or
      Drug-specific 5 half-lives elimination period (if longer than 4 weeks).
   g. Any medication that, in the opinion of the Investigator, would interfere with evaluation of the VIB4920 or interpretation of subject safety or study results.
   h. Any increase or initiation of new doses of cevimeline or pilocarpine and cyclosporine eye drops (Restasis) within 2 weeks prior to signing the ICF through randomization (Day 1).
18. Subjects who have received previous treatment with anti-CD40L compounds at any time before screening.
19. Subjects with blood tests, at screening, of any of the following:
   Aspartate aminotransferase (AST)>2×upper limit of normal (ULN)
   Alanine aminotransferase (ALT)>2×ULN
   Total bilirubin (TBL)>2×ULN
   Hemoglobin<75 g/L
   Neutrophils<1.0×109/L
   Platelets<100×109/L
   Prothrombin or partial thromboplastin time (PTT)>ULN

### Inclusion Criteria for Population #2

To be included in Population #2 of this study, each individual satisfied all the following criteria.
1. Adults, 18 years or older at time of informed consent (the minimum age for adult participants can be higher than 18 years in countries with different regulations).
2. Diagnosed with SS by meeting the 2016 ACR/EULAR Classification Criteria.
3. Have an ESSPRI score of ≥5 at screening.
4. Have an ESSDAI score of ≤5 at screening.
5. Positive for either anti-Ro autoantibodies or RF, or both at screening, as per the definition of the standard central laboratory test available.
6. Residual salivary gland function as defined by whole stimulated salivary flow>0.1 mL/min.
7. Written informed consent and any locally required authorization (e.g., Health Insurance Portability and Accountability Act in the United States, EU Data Privacy Directive in the EU) obtained from the subject/legal representative prior to performing any protocol-related procedures, including screening evaluations.
8. Females of childbearing potential who are sexually active with a nonsterilized male partner must use a highly effective method of contraception from signing the ICF and must agree to continue using such precautions through the end of the study follow-up; cessation of contraception after this point should be discussed with a responsible physician. Highly effective methods of contraception include:
   combined (estrogen and progestogen containing) hormonal contraception associated with inhibition of ovulation:
      oral
      intravaginal
      transdermal
   progestogen-only hormonal contraception associated with inhibition of ovulation:
      oral
      injectable
      implantable
   intrauterine device (IUD)
   intrauterine hormone-releasing system (IUS)
   bilateral tubal occlusion
   vasectomized partner
   sexual abstinence
Sexual abstinence is considered a highly effective method only if it is the preferred and usual lifestyle of the subject and the subject agrees to refrain from heterosexual intercourse from signing the ICF through the end of the study follow-up. Periodic abstinence, the rhythm method, and the withdrawal method are not acceptable methods of contraception. A recommendation that the female partners (of childbearing potential) of male study participants should use a highly effective method of contraception other than a barrier method is made.
   Females of childbearing potential are defined as those who are not surgically sterile (surgical sterilization includes bilateral tubal ligation, bilateral oophorectomy, or hysterectomy) or those who are not postmenopausal (defined as 12 months with no menses without an alternative medical cause).
   Vasectomized partner is a highly effective birth control method provided that partner is the sole sexual partner of the woman of childbearing potential trial participant and that the vasectomized partner has received medical assessment of the surgical success.
9. Non-sterilized male subjects who are sexually active with a female partner of childbearing potential must use a condom with spermicide from Day 1 through to the end of the study.
10. Meets all of the following TB criteria:
   i. No history of latent or active TB prior to screening, with the exception of latent TB with documented completion of appropriate treatment.
   j. No signs or symptoms suggestive of active TB from medical history or physical examination.
   k. No recent (≤12 weeks of screening) close contact with a person with active TB (close contact is defined as ≥4 hours/week OR living in the same household OR in a house where a person with active TB is a frequent visitor).
   l. Negative IGRA test result for TB obtained within 12 weeks prior to randomization. Subjects with an indeterminate test result can repeat the test, but if the repeat test is also indeterminate, they are excluded.
   m. A chest radiograph (obtained during the screening period or anytime within 12 weeks prior to signing of the ICF) with no evidence of current active TB or other infection, or old active TB, malignancy, or clinically significant abnormalities suggesting an active process (unless due to SS).

### Exclusion Criteria for Population #2

If an individual for Population #2 met any of the following criteria, he or she was ineligible:
1. Patients with medical history of confirmed deep venous thrombosis or arterial thromboembolism within 2 years of signing the ICF.
2. Patients with risk factors for venous thromboembolism or arterial thrombosis (e.g., immobilization or major surgery within 12 weeks before screening), prothrombotic status (including, but not limited to, congenital or inherited deficiency of antithrombin III, protein C, protein S, or confirmed diagnosis of catastrophic antiphospholipid syndrome).
3. Patients requiring treatment with anticoagulant drugs (clopidogrel, prasugrel, warfarin, low molecular weight heparin, etc.). Low-dose aspirin treatment (up to 325 mg/day) is allowed.
4. Concomitant polymyositis or dermatomyositis or systemic sclerosis.
5. Active malignancy or history of malignancy, except as follows:
   n. In situ carcinoma of the cervix treated with apparent success with curative therapy >12 months prior to screening; or
   o. Cutaneous basal cell carcinoma treated with apparent success with curative therapy
6. Subjects who are pregnant or lactating or planning to get pregnant during the duration of the study.
7. Subjects who have a positive test for, or have been treated for, hepatitis B, hepatitis C, or HIV infection.
   Regarding hepatitis B, a positive test for chronic hepatitis B infection at screening is defined as detection of either (1) hepatitis B surface antigen (HBsAg); or (2) hepatitis B core antibody (anti-HIBc).
8. Subjects with:
   a. A history of more than one episode of herpes zoster and/or opportunistic infections in the last 12 months, with the exception of oral candidiasis, vaginal candidiasis, and cutaneous fungal infections.
   b. Active viral, bacterial or other infections requiring systemic treatment at the time of screening or through randomization, or history of more than 2 infections requiring IV antibiotics within 12 months prior to signing the ICF.
   c. Epidemiologic risk of COVID-19 (recent exposures, high-risk housing) and for health-related risk of COVID-19 severity based on current understanding of risk factors for severe disease when making a decision regarding the individual subject's risk of participation. Subjects who have active COVID-19 infection or disease or other significant infection, or, in the judgment of the investigator, who may be at unacceptable risk of COVID-19, or its complications should not be randomized.
   d. A documented positive SARS-CoV-2 test within 2 weeks prior to randomization. Subjects with a positive test for SARS-CoV-2 may be rescreened at least 2 weeks after a positive test if asymptomatic and at least 3 weeks after symptomatic COVID-19 illness.
9. Subjects with known history of severe allergy or reaction to any component of VIB4920 formulation or to any other biologic therapy.
10. Subjects with any severe cardiovascular, respiratory, endocrine, gastrointestinal, hematological, neurological, psychiatric, or systemic disorder or any other condition that, in the opinion of the Investigator would interfere with evaluation of the VIB4920 or interpretation of subject safety or study results.
11. Subjects who are unable or unwilling to comply with protocol requirements (e.g., active drug or alcohol abuse).
12. Subjects who have received live (attenuated) vaccine within the 4 weeks before ICF signature.
13. Last administration of experimental biologic (other than those listed in Point 14) or oral agents<3 months or 5 half-lives before randomization.
14. Subjects who have had previous treatment with any biologic B cell-depleting therapy (e.g., rituximab, ocrelizumab, ofatumumab) within 12 months or other B-cell targeting therapy (e.g., belimumab)<3 months before randomization.
15. Use of the following medications:
   a. Antimalarials (e.g., chloroquine, hydroxychloroquine, quinacrine) if they have been initiated or if the dose has changed within 8 weeks prior to signing the ICF or during the screening period.
   b. Oral, intramuscular, IV, or intra-articular corticosteroids within 4 weeks prior to signing the ICF through randomization (Day 1).
   c. MTX, AZA, leflunomide, other cDMARD, or immunosuppressive or antiproliferative medications, if last dose was taken within:
      4 weeks prior to signing ICF or
      Drug-specific 5 half-lives elimination period (if longer than 4 weeks).
   d. Any medication that in the opinion of the Investigator would interfere with evaluation of VIB4920 or interpretation of subject safety or study results
   e. Any increase or initiation of a new dose of regularly scheduled nonsteroidal anti-inflammatory drugs within 2 weeks prior to signing the ICF through randomization (Day 1).
   f. Any increase or initiation of new doses of cevimeline or pilocarpine and cyclosporine eye drops (Restasis) within 2 weeks prior to signing the ICF through randomization (Day 1).
16. Subjects who have received previous treatment with anti-CD40L compounds at any time before screening.
17. Subjects with blood tests, at screening, of any of the following:

$$AST > 2 \times ULN$$

$$ALT > 2 \times ULN$$

$$TBL > 2 \times ULN$$

$$Hemoglobin < 75 g / L$$

$$Neutrophils < 1. \times 109 / L$$

$$Platelets < 100 \times 109 / L$$

$$Prothrombin or PTT > ULN$$

Safety-related screening assessments included adverse events (AEs), safety laboratory tests (serum chemistry, hematology, and urinalysis), coagulation parameters, chest X-ray, thyroid function tests, and serum beta-human chorionic gonadotropin (β-hCG) pregnancy test for females of childbearing potential.

### Safety Assessment

Safety assessments were conducted during the active treatment period according to the assessments shown in Table 5 and consisted of:
Monitoring and recording all Adverse Events (AEs) (including Adverse Events of Special Interest) and Serious Adverse Events
Safety laboratory tests (serum chemistry, hematology, and urinalysis)
Concomitant medications
Vital signs, physical examination (full or symptom-driven depending on the visit), and weight
ECG
Urine pregnancy test (for females of childbearing potential)
Coagulation tests

### Adverse Event (AE)

An AE is any untoward medical occurrence associated with the use of an intervention in humans whether or not it is considered intervention-related.

### Serious Adverse Event (SAE)

An SAE is considered “serious” if it results in any of the following outcomes:
Death
A life-threatening AE (An event is considered “life-threatening” if, in the view of either the Investigator or Sponsor, its occurrence places the patient or subject at immediate risk of death. It does not include an AE or suspected adverse reaction (SAR) that, had it occurred in a more severe form, might have caused death.)
Inpatient hospitalization or prolongation of existing hospitalization
A persistent or significant incapacity or substantial disruption of the ability to conduct normal life functions
A congenital anomaly/birth defect
Important medical events that may not result in death, be life-threatening, or require hospitalization may be considered serious when, based upon appropriate medical judgment, they may jeopardize the patient or subject and may require medical or surgical intervention to prevent one of the outcomes listed in this definition. Examples of such medical events include allergic bronchospasm requiring intensive treatment in an emergency room or at home, blood dyscrasias or convulsions that do not result in inpatient hospitalization, or the development of drug dependency or drug abuse.

### Causality or Relatedness

An assessment of the relationship of AEs and SAEs to VIB4920 was determined. An event was considered “not related” to use of VIB4920 if any of the following tests were met:
a. An unreasonable temporal relationship between administration of VIB4920 and the onset of the event (e.g., the event occurred either before, or too long after, administration of VIB4920 for it to be considered VIB4920-related)
b. A causal relationship between the VIB4920 and the event is biologically implausible (e.g., death as a passenger in an automobile accident)
c. A clearly more likely alternative explanation for the event is present (e.g., typical adverse reaction [AR] to a concomitant drug and/or typical disease-related event)

Individual AE/SAE reports were considered “related” to use of VIB4920 if the “not related” criteria was not met. “Related” implies that the event was considered to be “associated with the use of the drug” meaning that there is “a reasonable possibility” that the event may have been caused by the product (i.e., there are facts, evidence, or arguments to suggest possible causation).

### Severity/Intensity

Severity was assessed according to the following scale: Grade 1: An event of mild intensity that is usually transient and may require only minimal treatment or therapeutic intervention. The event does not generally interfere with usual activities of daily living. Grade 2: An event of moderate intensity that is usually alleviated with additional, specific therapeutic intervention. The event interferes with usual activities of daily living, causing discomfort but poses no significant or permanent risk of harm to the subject. Grade 3: A severe event that requires intensive therapeutic intervention. The event interrupts usual activities of daily living, or significantly affects the clinical status of the subject. Grade 4: An event, and/or its immediate sequelae, that is associated with an imminent risk of death or with physical or mental disabilities that affect or limit the ability of the subject to perform activities of daily living (eating, ambulation, toileting, etc.). Grade 5: Death (loss of life) as a result of an event.

### Adverse Events of Special Interest (AESI)

Adverse events of special interest (AESI) were evaluated. An AESI is one of scientific and medical interest specific to understanding of VIB4920 and may require close monitoring and collection of additional information. An AESI may be serious or nonserious.

The following AESIs were monitored:
Thrombotic and embolic events
   Hepatic function abnormality (meeting the definition of Hy's Law (HL))
   Anaphylaxis and clinically significant (Grade 3 or higher) hypersensitivity reactions
   Severe Infusion-related reactions (Common Terminology Criteria for Adverse Events (CTCAE) Grade 3 or higher).
   Severity was assessed according to CTCAE v5
      Grade 1: Mild transient reaction; infusion interruption not indicated; intervention not indicated.
      Grade 2: Therapy or infusion interruption indicated but responds promptly to symptomatic treatment (e.g., antihistamines, NSAIDS, narcotics, IV fluids); prophylactic medications indicated for ≤24 hours
      Grade 3: Prolonged (e.g., not rapidly responsive to symptomatic medication and/or brief interruption of infusion); recurrence of symptoms following initial improvement; hospitalization indicated for clinical sequelae
      Grade 4: Life-threatening consequences; urgent intervention indicated
   Malignant neoplasm
   Immune complex disease
   Infections:
      Clinically significant (Grade 3 or higher)
      Opportunistic infections including but not limited to reactivation of latent viral infection, invasive fungal infections, and TB
         Table 3. summarizes the screening procedures for the study. More than one visit might be needed to complete screening.

TABLE 3
Screening Procedures

| | Study Period |
|---|---|
| | Screening |
| | Visit Number |
| | V1 |
| | Study Day |
| | Day −28 |
| Procedure | to Day −1 |
| Written informed consent | X |
| SID number assignment | X |
| Medical history | X |
| 2016 ACR/EULAR Criteria for classification of SS | X |
| Whole stimulated salivary flow | X |
| Schirmer's test^{a} | X |
| ESSDAI | X |
| ESSPRI | X |
| Assessment of prior and concomitant medications | X |
| Verify eligibility criteria | X |
| Complete physical examination (including weight | X |
| and height) | |
| 28-joint count | X |
| ECG | X |
| Vital signs | X |
| Assessment of AEs/SAEs | X |
| Virology: Hepatitis B, C; HIV-1; HIV-2, | X |
| SARS-CoV-2^{b} | |
| TB test (IGRA)^{c} | X |
| Safety Lab Tests: Serum Chemistry, Hematology, | X |
| Urinalysis | |
| Baseline coagulation panel (prothrombin time, PTT) | X |
| Autoantibody panel (anti-SSA [Ro], anti-SSB | X |
| [La], ANA) | |
| Exploratory biomarker sample (serum) | X |
| Exploratory biomarker sample (plasma) | X |
| RF | X |
| ESSDAI-required blood tests (C3, C4, IgG, serum | X |
| and urine immunofixation, cryoglobulins, serum | |
| free light chains) | |
| Chest X-ray^{d} | X |
| Thyroid Function Tests (TSH, T3, T4, free T4) | X |
| Pregnancy test (serum β-hCG; females of | X |
| childbearing potential only) | |
| ActiGraph provided to subject (optional) | X |

AE = adverse event; ANA = antinuclear antibodies; APL = antiphospholipid; β-hCG = beta-human chorionic gonadotropin; C = complement; ECG = electrocardiogram; ESSDAI = European League Against Rheumatism (EULAR) Sjögren's Syndrome Disease Activity Index; ESSPRI = EULAR Sjögren's Syndrome Patient Reported Index; HIV = human immunodeficiency virus; IGRA = Interferon Gamma Release Assay; PTT = partial thromboplastin time; RF = rheumatoid factor; SAE = serious adverse event; SID = subject identification; SS = Sjögren's syndrome; TB = tuberculosis; TSH = thyroid stimulating hormone.
^{a}Schirmer's test optional; only conducted if needed to confirm SS diagnosis.
^{b}To participate in study, subjects required a negative SARS-CoV-2 test within 2 weeks prior to randomization.
^{c}IGRA not required during screening if performed within 12 weeks prior to screening visit with a documented negative result.
^{d}Chest X-ray not required during screening if performed within 12 weeks prior to screening visit.

### Dose Formulation

The formulation of VIB4920 is shown in Table 4.

TABLE 4
Exemplary formulation of VIB4920 and Placebo

| Product | Dose | Frequency | Route | Duration |
|---|---|---|---|---|
| VIB4920 | 1500 mg | Stage I: | IV | Up to 169 days |
| | | Q2W × 3 | | |
| | | Q4W × 4 | | |
| | | Stage II: | | |
| | | Q4W × 5 | | |
| VIB4920 | 0.9% (w/v) | Stage I: | IV | Up to 169 days |
| Placebo | saline | Q2W × 3 | | |
| | | Q4W × 4 | | |
| | | Stage II: | | |
| | | Q4W × 5 | | |

IV = intravenous; Q2W = once every 2 weeks; Q4W = once every 4 weeks; w/v = weight/volume.

VIB4920 is formulated at 100 mg/mL VIB4920 in 10 mM sodium phosphate buffer, 250 mM sucrose, 0.02% (weight/volume [w/v]) poloxamer 188, pH 7.4. The nominal volume in each vial is 5.0 mL. VIB4920 is a sterile liquid drug product (500 mg VIB4920 per vial, nominal) intended for IV infusion following dilution in normal saline. VIB4920 should not be shaken and requires no special biohazard handling. It must be stored at 2° C. to 8° C. (36° F. to 46° F.) in refrigerator with adequate temperature monitoring. VIB4920 must not be frozen.

Placebo was 0.9% (w/v) saline provided by the site as 250 mL prefilled IV bags.

### Treatment

Both Populations #1 and #2 received the following treatment regimen:
Treatment group 1 received 1500 mg VIB4920 as an IV infusion, Q2W×3, then Q4W for 4 additional doses (Stage I). Starting on Day 169 subjects received placebo Q4W for 5 doses (Stage II).
Treatment group 2 received placebo as an IV infusion Q2W×3, then Q4W for 4 additional doses (Stage I). Starting on Day 169 subjects received VIB4910 1500 mg Q4W for 5 doses (Stage II).

The VIB4920 was infused using an IV infusion pump. During Stage I each subject received the entire volume of VIB4920 solution in the IV bag over at least 90 minutes (approximately 2.8 mL/min). The VIB4920 was infused through a low-protein binding 0.2- or 0.22-line filter. Vital signs were obtained prior to the start of each IP infusion. For Stage II, infusions were administered over at least 60 minutes (approximately 4.2 mL/min).

On the first 2 dosing days of both Stage I (Day 1 and Day 15) and Stage II (Day 169 and Day 197) vital signs were measured within 30 minutes prior to the start of infusion, every 30 minutes (±5 minutes) during the infusion, at the end of the infusion (+5 minutes), then at 1 hour and 2 hours after the end of infusion (±10 minutes). On subsequent dosing days, vital signs were measured within 30 minutes prior to the start of infusion, every 30 minutes (±5 minutes), at the end of the infusion (+5 minutes) and 30 minutes after the end of the infusion (±5 minutes). At the end of these observation periods, subjects were discharged if they were stable.

### Post-Treatment Assessment

Post-treatment, subjects in Population #1 were assessed to determine one or more of:
Change from baseline in ESSDAI at Days 85, 169, and 309
Change from baseline in ESSPRI at Days 85, 169, and 309
Proportion of subjects achieving ESSDAI (i.e., decrease of at least 3 points) and ESSDAI [4](i.e., decrease of at least 4 points) response, defined as a decrease of at least 3[4] points from baseline in the ESSDAI at Days 85, 169, and 309 without premature discontinuation from the study and without receiving rescue therapy
Change from baseline in Functional Assessment of Chronic Illness Therapy (FACIT)-Fatigue score at Days 85, 169, and 309
Change from baseline in Ocular Surface Disease Index (OSDI©) at Days 85, 169, and 309
Patient's Global Impression of Severity (PGIS) at Days 85, 169, and 309
Safety and tolerability of multiple IV doses of VIB4920 as measured by the incidence of treatment-emergent adverse events (TEAEs), treatment-emergent serious adverse events (TESAEs), adverse events of special interest (AESIs), and laboratory, vital sign, and electrocardiogram (ECG) abnormalities
PK during the study
Proportion of subjects with positive immunogenic response measured by anti-VIB4920 antibodies until the completion of the study
Change from baseline in Total Stimulated Salivary Flow
Change from baseline in Schirmer's test
Change from baseline in SS-A, SS-B and IgG and IgM rheumatoid factor (RF) autoantibodies
Patient's Global Impression of Change (PGIC) at Day 169
Change from baseline in Physician's Global Impression of Severity
Change from baseline in levels of markers of inflammation (immunoglobulins, beta-2 microglobulin, C-reactive protein [CRP], CXCL13, C3, C4 and serum free light chains)
Proportion of subjects achieving response in composite index, defined as either ESSDAI-3 improvement or ESSPRI-1 improvement at Days 85, 169, and 309 plus no worsening of Physician's Global Impression of Severity from baseline
Change from baseline in SF36 Physical Component Score and Mental Component Score
Visual analog scale (VAS) oral dryness
VAS ocular dryness
Patient-Reported Outcomes Measurement Information System® (PROMIS-29 Profile v2.1)
Biomarkers including blood flow, cytometry for changes in T and B-cell subsets, and salivary proteins
Blood gene expression.
Changes in fecal microbiome (optional)
DNA epigenetics at baseline and post-treatment (optional)
Actigraphy: overall activity and changes in sleep patterns during the study (optional)
Change from baseline in Clinical EULAR Sjögren's Syndrome Disease Activity Index (ClinESSDAI) at Days 85, 169, and 309
Proportion of subjects achieving ClinESSDAI and ClinESSDAI response, defined as a decrease of at least 3[4] points from baseline in the ClinESSDAI at Days 85, 169, and 309 without premature discontinuation from the study and without receiving rescue therapy in order to:
1. Evaluate the clinical efficacy of multiple doses of VIB4920 in glandular and extra glandular manifestations of SS patients with moderate to high systemic disease activity
2. Evaluate the effect of VIB4920 on systemic activity and patient-reported outcomes in subjects with SS
3. Evaluate the safety and tolerability of multiple doses of VIB4920 in subjects with SS
4. Characterize the pharmacokinetics (PK) of VIB4920 in subjects with SS
5. Assess the immunogenicity of VIB4920 in subjects with SS
6. Evaluate the pharmacodynamics of VIB4920 on CD40/CD40L pathways and disease biomarkers in subjects with SS
7. Evaluate changes in T and B-cell populations and biomarkers of inflammation and autoantibodies in subjects with SS upon treatment with VIB4920
8. Evaluate the effects of VIB4920 on systemic disease activity and salivary and lacrimal function in subjects with SS
9. Evaluate changes in key subjective complaints associated with SS

Post-treatment, subjects in Population #2 were assessed to determine one or more of.
Change from baseline in ESSPRI at Days 85, 169, and 309
Proportion of subjects achieving ESSPRI response, defined as ≥1 point or 15% reduction from baseline in ESSPRI score at Days 85, 169, and 309 without premature discontinuation from the study and without receiving rescue therapy
Change from baseline in FACIT-Fatigue at Days 85, 169, and 309
Change from baseline in OSDI at Days 85, 169, and 309
PGIS at Days 85, 169, and 309
Safety and tolerability of multiple IV doses of VIB4920 as measured by the incidence of TEAEs, TESAEs, AESIs, and laboratory, vital sign, and ECG abnormalities
PK during the study
Proportion of subjects with positive immunogenic response measured by anti-VIB4920 antibodies until the completion of the study
Change from baseline in ESSDAI
Change from baseline in Total Stimulated Salivary Flow
Change from baseline in Schirmer's test
Change from baseline in SS-A, SS-B and IgG and IgM RF autoantibodies
PGIC at Day 169
Change from baseline in Physician's Global Impression of Severity
Change in levels of markers of inflammation from baseline (immunoglobulins, beta-2 microglobulin, CRP, CXCL13, C3, C4, and serum free light chains)
Proportion of subjects achieving response in composite index, defined as either ESSDAI-3 improvement or ESSPRI-1 improvement at Days 85, 169, and 309 plus no worsening of Physician's Global Impression of Severity from baseline
Change from baseline in SF36 Physical Component Score and Mental Component Score
VAS oral dryness
VAS ocular dryness
PROMIS-29 Profile v2.1
Biomarkers including blood flow, cytometry for changes in T- and B-cell subsets, and salivary proteins
Blood gene expression.
Changes in fecal microbiome (optional)
DNA epigenetics at baseline and post-treatment (optional)
Actigraphy: overall activity and changes in sleep patterns during the study (optional)
Change from baseline in ClinESSDAI at Days 85, 169, and 309
   in order to:
1. Evaluate the clinical efficacy of multiple doses of VIB4920 in the key subjective complaints of SS (dryness, fatigue, and pain)
2. Evaluate the effect of VIB4920 on patient-reported outcomes in subjects with SS
3. Evaluate the safety and tolerability of multiple doses of VIB4920 in subjects with SS
4. Characterize the PK of VIB4920 in subjects with SS
5. Assess the immunogenicity of VIB4920 in subjects with SS
6. Evaluate the pharmacodynamics of VIB4920 on CD40/CD40L pathways and disease biomarkers in subjects with SS
7. Evaluate changes in T and B-cell populations and biomarkers of inflammation and autoantibodies in subjects with SS upon treatment with VIB4920
8. Evaluate the effects of VIB4920 on systemic disease activity and salivary and lacrimal function in subjects with SS
9. Evaluate changes in key subjective complaints associated with SS

### Clinical Laboratory

Post-treatment, blood and urine samples were collected for laboratory safety tests. A hematology panel included a complete blood count, with differential (including basophils, eosinophils, lymphocytes, monocytes, and neutrophils), hemoglobin, hematocrit, platelets and white blood cell count.

Serum chemistry was analyzed for:
Creatinine, blood urea nitrogen, fasting glucose, total protein, creatine kinase and electrolytes (including sodium, potassium, chloride, calcium, bicarbonate, and phosphorus)
Hepatic Profile: Albumin, TBL, indirect bilirubin, AST, ALT, alkaline phosphatase (ALP), and gamma-glutamyltransferase

Coagulation parameters were assessed. Subjects were evaluated for prothrombin time and PTT during screening and at all study visits during the treatment and off-treatment periods (Table 2 and Table 3, respectively).

The urinalysis included protein, glucose, blood, ketones, leukocytes, and pH by dipstick analysis. Microscopy (crystals, casts, white blood cells, red blood cells) were performed if any abnormalities were observed.

Testing for SARS-COV-2 was performed.

Vital signs, including systolic and diastolic blood pressure (mmHg), pulse rate (beats/min), respiratory rate (breaths/min), body temperature (° C.) and body weight (kg) were measured.

A 12-lead ECG were performed during screening, treatment period, and off-treatment periods. Each ECG included ventricular heart rate and intervals (PR, QRS, QT, QTc).

Serum β-hCG pregnancy test(s) were completed for all females of childbearing potential during the screening period (before Day 0) and by urine pregnancy test at the visits during the treatment period and off-treatment period.

### Safety Assessment

Adverse events (AEs) and serious adverse events (SAEs)

The schedules of study assessments for the active treatment period and for the off-treatment period are presented in Table 5 and Table 6, respectively. Table 6 also summarizes all assessments that could be conducted at any unscheduled visit during the study.

TABLE 5
Exemplary schedule of study assessments during the treatment period

| | Visit number | | | | | | | | | | | |
|---|---|---|---|---|---|---|---|---|---|---|---|---|
| | | | | | | | | V9 | | | | |
| | V2 | V3 | V4 | V5 | V6 | V7 | V8 | Stage I/ | V10 | V11 | V12 | V13 |
| | Stage I | | | | | | | Stage II^{a} | Stage II | | | |
| Study day | 1 | 15 ± | 29 ± | 57 ± | 85 ± | 113 ± | 141 ± | EP: 169 ± | 197 ± | 225 ± | 253 ± | 281 ± |
| Procedure | Baseline | 1 d | 3 d | 3 d | 3 d | 3 d | 3 d | 3 d | 3 d | 3 d | 3 d | 3 d |
| Verify | X | | | | | | | | | | | |
| eligibility | | | | | | | | | | | | |
| criteria | | | | | | | | | | | | |
| Randomization | X | | | | | | | | | | | |
| ESSPRI | X | | X | X | X | X | X | X | X | X | X | X |
| SF-36 v2 | X | | X | X | X | X | X | X | X | X | X | X |
| Questionnaires | | | | | | | | | | | | |
| FACIT-Fatigue | X | | X | X | X | X | X | X | X | X | X | X |
| Questionnaire | | | | | | | | | | | | |
| VAS | X | | X | X | X | X | X | X | X | X | X | X |
| Oral/Ocular | | | | | | | | | | | | |
| PROMIS-29 | X | | X | X | X | X | X | X | X | X | X | X |
| Profile v2.1 | | | | | | | | | | | | |
| OSDI | X | | X | X | X | X | X | X | X | X | X | X |
| PGIC | | X | X | X | X | X | X | X | X | X | X | X |
| PGIS | X | | X | X | X | X | X | X | X | X | X | X |
| Stimulated | X | | | | X | | | X | | | X | |
| salivary flow | | | | | | | | | | | | |
| measurement | | | | | | | | | | | | |
| Schirmer's test | X | | | | X | | | X | | | X | |
| ESSDAI | X | | X | X | X | X | X | X | X | X | X | X |
| Assessment of | X | X | X | X | X | X | X | X | X | X | X | X |
| AEs/SAEs | | | | | | | | | | | | |
| Concomitant | X | X | X | X | X | X | X | X | X | X | X | X |
| medications | | | | | | | | | | | | |
| Full physical | X | | | | | | | X | | | | X |
| examination | | | | | | | | | | | | |
| Symptom- | | X | X | X | X | X | X | | X | X | X | |
| driven physical | | | | | | | | | | | | |
| exam^{b} | | | | | | | | | | | | |
| Vital Signs | X | X | X | X | X | X | X | X | X | X | X | X |
| (BP, HR, RR | | | | | | | | | | | | |
| and temp) | | | | | | | | | | | | |
| ECG | X | | | | | | | X | | | | |
| Weight | X | | | | | | | X | | | | X |
| 28-joint | X | | X | X | X | X | X | X | X | X | X | X |
| assessment | | | | | | | | | | | | |
| Physician | X | X | X | X | X | X | X | X | X | X | X | X |
| Global | | | | | | | | | | | | |
| Impression of | | | | | | | | | | | | |
| Severity | | | | | | | | | | | | |
| Safety lab tests^{c} | X | X | X | X | X | X | X | X | X | X | X | X |
| Urine | X | | X | X | X | X | X | X | X | X | X | X |
| pregnancy test | | | | | | | | | | | | |
| in women of | | | | | | | | | | | | |
| childbearing | | | | | | | | | | | | |
| potential | | | | | | | | | | | | |
| Coagulation | X | X | X | X | X | X | X | X | X | X | X | X |
| tests | | | | | | | | | | | | |
| (prothrombin | | | | | | | | | | | | |
| time, PTT) | | | | | | | | | | | | |
| Autoantibody | | | | | X | | | X | | | | |
| panel^{d} | | | | | | | | | | | | |
| RF | X | X | X | X | X | X | X | X | X | X | X | X |
| Inflammatory | X | X | X | X | X | X | X | X | X | X | | X |
| markers^{e} | | | | | | | | | | | | |
| C3, C4, serum | X | X | X | X | X | X | X | X | X | X | X | X |
| free light | | | | | | | | | | | | |
| chains, serum | | | | | | | | | | | | |
| and urine | | | | | | | | | | | | |
| immunofixation | | | | | | | | | | | | |
| Exploratory | X | X | X | X | X | | X | X | X | X | X | X |
| Flow | | | | | | | | | | | | |
| Cytometry | | | | | | | | | | | | |
| sample (whole | | | | | | | | | | | | |
| blood) | | | | | | | | | | | | |
| Exploratory | X | X | X | X | X | | X | X | X | X | X | X |
| biomarker | | | | | | | | | | | | |
| sample (serum) | | | | | | | | | | | | |
| Exploratory | X | X | X | X | X | | X | X | X | X | X | X |
| biomarker | | | | | | | | | | | | |
| sample | | | | | | | | | | | | |
| (plasma) | | | | | | | | | | | | |
| Saliva for | X | | | | X | | | X | | | X | |
| protein and | | | | | | | | | | | | |
| autoantibody | | | | | | | | | | | | |
| biomarkers | | | | | | | | | | | | |
| ADA (plasma) | X | | X | | X | | | X | X | | X | |
| RNA PAXgene | X | X | X | X | X | | X | X | X | X | X | X |
| (whole blood)^{f} | | | | | | | | | | | | |
| PK (plasma) | X^{g} | X | X | X | X | X | X^{g} | X^{g} | X | X | X | X |
| DNA | X | | | | X | | | X | | | X | |
| (epigenetics) | | | | | | | | | | | | |
| (optional)^{f} | | | | | | | | | | | | |
| Fecal | X | | | | | | | X | | | | |
| Microbiome | | | | | | | | | | | | |
| sample | | | | | | | | | | | | |
| (optional)^{f} | | | | | | | | | | | | |
| Qualitative | | | | | | | | X | | | | |
| Patient | | | | | | | | | | | | |
| Interview | | | | | | | | | | | | |
| (optional)^{h} | | | | | | | | | | | | |
| Administration | X | X | X | X | X | X | X | X | X | X | X | X |
| of IP | | | | | | | | | | | | |
| (VIB4920/ | | | | | | | | | | | | |
| placebo) | | | | | | | | | | | | |

ADA = anti-drug antibodies; AE = adverse event; ANA = antinuclear antibody; BP = blood pressure; C = complement; d = day(s); ECG = electrocardiogram; EP = endpoint; ESSDAI = EULAR Sjögren's Syndrome Disease Activity Index; ESSPRI = EULAR Sjögren's Syndrome Patient Reported Index; FACIT-Fatigue = Functional Assessment of Chronic Illness Therapy -Fatigue; HR = heart rate; IP = investigational product; OSDI = Ocular Surface Disease Index; PGIC = Patient Global Impression of Change; PGIS = Patient Global Impression of Severity; PK = pharmacokinetic; PROMIS-29 Profile v2.1 = Patient-Reported Outcomes Measurement Information System ®; PTT = partial thromboplastin time; RF = rheumatoid factor; RR = respiratory rate; SAE = serious adverse event; SF-36 v2 = 36-Item Short Form Survey version 2; V = Visit; VAS = visual analog scale.
Note:
All laboratory sample collections and assessments on dosing day must be performed at pre-dose, unless specified otherwise.
^{a}Day 169 is primary endpoint for Stage I and first day of IP administration for Stage II.
^{b}If no symptoms are present, the exam can be limited to assess ESSDAI.
^{c}Safety laboratory tests: Serum chemistry, hematology and urinalysis.
^{d}Autoantibody panel: anti-SSA (Ro), anti-SSB (La), and ANA.
^{e}Inflammatory markers: plasma immunoglobulin levels (IgM, IgG, IgA), beta-2 microglobulin, and high sensitivity C-reactive protein (CRP) (CXCL13 was assessed as an exploratory biomarker).
^{f}Not collected in China.
^{g}Plasma sample for PK to be collected pre- and postdose (at the end of infusion).
^{h}Telephone-based interview applicable to English-speaking subjects in the USA and UK only, and can be conducted anytime from Day 169 onwards.

TABLE 6
Exemplary schedule of assessments during the off-
treatment period and any unscheduled visits

| | Visit number | | |
|---|---|---|---|
| | | V15 and | |
| | V14 | EDV | |
| | Off-treatment Period | | |
| | D 309 ± | D 365 ± | Unscheduled |
| Study day Procedure | 7 d | 7 d | Visit^{a} |
| ESSPRI | X | X | |
| SF-36 v2 Questionnaires | X | X | |
| FACIT-Fatigue questionnaires | X | X | |
| VAS Oral/Ocular | X | X | |
| PROMIS-29 Profile v2.1 | X | X | |
| OSDI | X | X | |
| PGIC | X | X | |
| PGIS | X | X | |
| Stimulated salivary | X | X | |
| measurement | | | |
| Schirmer's test | X | X | |
| ESSDAI | X | X | |
| Assessment of AEs/SAEs | X | X | X |
| Concomitant medications | X | X | X |
| Full physical examination | X | X | |
| Symptom-driven physical | | | X |
| exam^{b} | | | |
| Vital Signs (BP, HR, RR | X | X | X |
| and temp) | | | |
| ECG | | X | |
| Weight | X | X | |
| 28-joint assessment | X | X | |
| Physician Global | X | X | |
| Impression of Severity | | | |
| Safety lab tests | X | X | X |
| Urine pregnancy test | X | X | X |
| Coagulation tests | X | X | X |
| RF | X | X | |
| Inflammatory markers | X | X | |
| C3, C4, serum free light chains, | X | X | |
| cryoglobulins, IgG, serum | | | |
| and urine immunofixation | | | |
| Exploratory Flow Cytometry | X | X | |
| sample (whole blood) | | | |
| Exploratory biomarker sample | X | X | |
| (serum) | | | |
| Exploratory biomarker sample | X | X | |
| (plasma) | | | |
| Saliva for antibody and | X | X | |
| autoantibody biomarkers | | | |
| ADA | X | X | |
| RNA PAXgene (whole blood)^{c} | X | X | |
| PK (plasma) | X | X | |
| DNA (epigenetics) (optional)^{c} | X | | |
| Fecal microbiome (optional)^{c} | X | | |
| ActiGraph collected from | | X | |
| subject (optional) | | | |

ADA = anti-drug antibodies; AE = adverse event; BP = blood pressure; C = complement; D = Day; d = days; ECG = electrocardiogram; EDV = early discontinuation visit; ESSDAI = EULAR Sjögren's Syndrome Disease Activity Index; ESSPRI = EULAR Sjögren's Syndrome Patient Reported Index; FACIT-Fatigue = Functional Assessment of Chronic Illness Therapy -Fatigue; HR = heart rate; OSDI = Ocular Surface Disease Index; PGIC = Patient Global Impression of Change; PGIS = Patient Global Impression of Severity; PK = pharmacokinetic; RF = rheumatoid factor; RR = respiratory rate; SAE = serious adverse event; SF-36 v2 = 36-Item Short Form Survey version 2; V = Visit; VAS = visual analog scale.
^{a}The assessments at any unscheduled visits may be adjusted as clinically indicated.
^{b}If no symptoms are present, the exam can be limited to assess ESSDAI.
^{c}Not collected in China.

### Results for Subjects with Moderate-to-High Systemic Disease Activity (as Defined by ESSDAI), “Population 1”

Results for change from baseline in ESSDAI at Day 169 and an analysis of responders are provided in Tables 7 and 8, respectively. Data in Table 7 shows that at week 24, subjects treated with Dazodalibep achieved a 6.3±0.6-point reduction in their ESSDAI score and subjects treated with placebo achieved a 4.1±0.6-point reduction resulting in a statistically significant least squares mean difference of 2.2 points (p=0.017). Dazodalibep achieved the primary endpoint with statistical significance in subjects with moderate-to-high systemic disease activity as defined by ESSDAI, which represents a significant achievement in the development of a treatment for Sjögren's syndrome, a disease with no FDA-approved treatments available.

Table 7 shows results for change from
baseline in ESSDAI at day 169

| n | 36 | 33 |
|---|---|---|
| LSMean (SE) | −4.1 (0.6) | −6.3 (0.6) |
| LSMean difference | | −2.2 |
| 90% CI | | (−3.6, −0.7) |
| P-value | | 0.0167 |

In addition to the primary endpoint analysis above, numerical improvements were seen in key secondary, exploratory and post-hoc analyses. These included measures of dryness, which is an important symptom for subjects living with Sjögren's syndrome as it impacts chewing, swallowing and dentition. Fatigue as measured by Functional Assessment of Chronic Illness Therapy-Fatigue (FACIT-F) and physical functioning measured using the 36-Item Short Form Health Survey (SF-36) showed numerical improvements, as did the number of tender and swollen joints. In addition, a post-hoc responder analysis of subjects achieving high levels of improvement on ESSDAI favored Dazodalibep over placebo. Additionally, Dazodalibep was well tolerated in the trial.

Further, compared to the placebo group, numerically greater improvement in the Dazodalibep group was observed via: the EULAR Sjögren's Syndrome Patient Reported Index score, Functional Assessment of Chronic Illness Therapy-Fatigue score, 36-Item Short Form Health Survey Physical Component Score, and stimulated salivary flow rate. Additionally, a post-hoc responder analysis of subjects achieving high levels (5 and 6 points) of improvement on ESSDAI favored Dazodalibep over placebo.

Table 8 shows responder analysis results

| Day 169 | Placebo | HZN4920 | Delta | P-value |
|---|---|---|---|---|
| ESSDAI-3 | 59.5% | 72.2% | 12.7% | 0.3283 |
| ESSDAI-4 | 48.6% | 66.7% | 18.1% | 0.1823 |
| ESSDAI-5 | 35.1% | 61.1% | 26.0% | 0.0449 |
| ESSDAI-6 | 34.3% | 60.0% | 25.7% | 0.0462 |

### Example 2—a Prophetic Phase III Study to Evaluate the Efficacy and Safety of VIB4920 in Subjects with Sjögren's Syndrome (SS)

A study is conducted that will evaluate the efficacy and safety of VIB4920 when administered to a subject with Sjögren's syndrome (SS). This study will evaluate the effect of VIB4920 over placebo in systemic manifestations of SS patients with moderate to high systemic disease activity using 2 different dosing regimens.

### Study Design

The study will enroll Sjögren's disease patients of 2 populations: Population #1 will comprise SS patients with moderate to high systemic activity, defined by ESSDAI≥5. The following domains are scored but they may not contribute to the minimum ESSDAI score of 5 required for inclusion: peripheral nervous system, central nervous system, and pulmonary. Population #2 will comprise SS patients with exocrine dysfunction and subjective symptoms defined by an ESSPRI score of ≥5, considered as the cut-off point for “unsatisfactory symptom state” but lower systemic disease activity but with lower systemic disease activity (with ESSDAI score≤5).

A complete physical examination, including weight, height, and 28-joint assessment is conducted.

### Dosing Schedule

Subjects will be divided among three dosages: Dose 1, 1500 mg VIB4920 every 2 weeks for 3 doses, then every 4 weeks thereafter; Dose 2, 3000 mg VIB4920 at week 1, week 4, and week 12, and then every 12 weeks thereafter; and placebo. The expected full duration of each subject's participation in this study is 392±7 days. An exemplary dosing schedule is provided in Table 9.

TABLE 9
Exemplary Dosing Schedule

| | |
|---|---|
| Arm 1 | 1500 mg Q4W + loading dose at W 2 |
| Arm 2 | 3000 mg Q12W + loading dose at W 4 |
| Arm 3 | Placebo |

### Example 3—CD40L Blockade with Dazodalibep (VIB4920) Reduces Blood Biomarkers of T and B Cell Costimulation in Subjects with Systemic Sjögren's Syndrome

Objective: To Evaluate the Impact of VIB4920 (Dazodalibep) on Blood Biomarkers of B and T Cell Costimulation in Adult SS Subjects with Moderate-to-High Systemic Disease Activity.

In several autoimmune diseases, the CD40/CD40L pathway is activated on a variety of cell types, including T cells, B cells, antigen-presenting cells, and activated epithelial cells. Dazodalibep blocks costimulatory signals between T cells and CD40-expressing B cells, disrupting the development of germinal centers, pathogenic B cells, plasma cells, and autoantibodies that are hallmarks of some autoimmune diseases such as Sjögren's Syndrome (SS).

A study was conducted that evaluated the efficacy of VIB4920 when administered to subjects with Sjögren's syndrome (SS). The study evaluated B cell subsets downstream of T cell stimulation: Ki67+CD27+ memory cells, CD27 high CD38 high plasmablasts, and CD11c high atypical memory cells. Serum CXCL13 concentrations, a chemokine involved in germinal center formation produced by activated follicular T cells, and rheumatoid factor (RF) autoantibodies was evaluated as well.

### Methods

A randomized, double-blind, placebo-controlled, crossover study to evaluate Dazodalibep therapy in adult SS subjects with moderate-to-high systemic disease activity, as defined by a EULAR Sjögren's Syndrome Disease Activity Index (ESSDAI) score≥5 was conducted. Subjects were randomized 1:1 to receive intravenous Dazodalibep 1500 mg or placebo (PBO) Q2W×3 doses, then Q4W×4 additional doses (Stage 1). Starting on Day 169, subjects initially randomized to Dazodalibep received PBO Q4W×5 doses and subjects initially randomized to PBO received Dazodalibep Q4W×5 doses and were then followed for 12 weeks (Stage 2). B cell subsets downstream of T cell stimulation (Ki67+CD27+ memory, CD27 high CD38 high plasmablasts, and CD11c high atypical memory cells) were assayed via FACS throughout the study period. Serum CXCL13 concentrations, a chemokine essential for germinal center (GC) formation produced by activated follicular T cells, and rheumatoid factor (RF) autoantibodies were also assayed.

### Results

Concomitant with Dazodalibep-related reductions in ESSDAI observed at Stage 1, significant and rapid reductions in Ki67+CD27+ memory B cells, plasmablasts, CD11c high memory B cells, CXCL13 and RF antibodies were observed from day 15 onwards in subjects who received Dazodalibep versus PBO. In Stage 2, similar reductions in these biomarkers were observed when PBO-treated subjects were transitioned to Dazodalibep treatment. In Dazodalibep-treated subjects who were transitioned to PBO in stage 2, these biomarkers returned to baseline values while sustained reductions in ESSDAI from baseline were observed through the duration of stage 2 in these subjects. Greater reductions in ESSDAI with Dazodalibep versus placebo were observed in independent subgroups of patients with elevated levels of Ki67+CD27+ memory B cells or CD11c high memory B cells at baseline.

Table 10 shows median fold change from baseline in placebo-Dazodalibep and Dazodalibep-placebo groups.

TABLE 10
The Impact of Dazodalibep on Blood Biomarkers of B and
T Cell Costimulation in Adult Sjogren's Syndrome Subjects
with Moderate-to-high Systemic Disease Activity

| | Days from study start | | | |
|---|---|---|---|---|
| Analyte Name | 85 | 169 | 253 | 365 |

Median (−/+ IQR) FC of baseline in Placebo-Dazodalibep group

| | | | | |
|---|---|---|---|---|
| CXCL13 (pg/mL) | 1.01 | 1.06 | 0.62 | 0.69 |
| | (0.87, 1.21) | (0.8, 1.24) | (0.45, 0.83) | (0.53, 0.85) |
| Rheumatoid | 1.23 | 1.27 | 0.55 | 0.45 |
| Factor (IU/mL) | (0.97, 1.31) | (1.13, 1.49) | (0.45, 0.92) | (0.38, 0.65) |
| % Ki-67+ (of post- | 1.07 | 1.32 | 0.58 | 0.47 |
| sw memory B cells) | (0.67, 1.68) | (0.69, 1.82) | (0.29, 1.05) | (0.26, 0.96) |
| % Plasmablasts | 1.19 | 0.89 | 0.58 | 0.57 |
| (of CD19 B cells) | (0.63, 1.87) | (0.68, 1.97) | (0.43, 1.12) | (0.33, 0.74) |
| % CD11cbr (of | 2.37 | 1.43 | 1.09 | 1.06 |
| CD19 B cells) | (1.19, 3.48) | (0.95, 2.64) | (0.7, 2.07) | (0.51, 4.29) |
| ESSDAI | 0.6 | 0.58 | 0.41 | 0.49 |
| Total Score | (0.34, 0.87) | (0.34, 0.84) | (0.18, 0.66) | (0.25, 0.71) |

Median (−/+ IQR) FC of baseline in Dazodalibep-Placebo group

| | | | | |
|---|---|---|---|---|
| CXCL13 (pg/mL) | 0.62 | 0.71 | 1.08 | 1.02 |
| | (0.55, 0.89) | (0.48, 0.85) | (0.84, 1.21) | (0.78, 1.15) |
| Rheumatoid | 0.41 | 0.29 | 0.97 | 0.91 |
| Factor (IU/mL) | (0.27, 0.74) | (0.27, 0.54) | (0.77, 1.21) | (0.81, 1.32) |
| % Ki-67+ (of post- | 0.45 | 0.44 | 1.59 | 1.14 |
| sw memory B cells) | (0.16, 0.63) | (0.25, 1) | (1.1, 2.36) | (0.94, 2.07) |
| % Plasmablasts | 0.56 | 0.61 | 1.85 | 1.84 |
| (of CD19 B cells) | (0.3, 0.72) | (0.34, 0.84) | (0.53, 3.11) | (0.72, 3.45) |
| % CD11cbr (of | 0.65 | 0.92 | 1.41 | 1.3 |
| CD19 B cells) | (0.47, 2.04) | (0.56, 2.01) | (0.81, 3.83) | (0.92, 2.91) |
| ESSDAI | 0.6 | 0.46 | 0.5 | 0.42 |
| Total Score | (0.41, 0.85) | (0.26, 0.57) | (0.34, 0.67) | (0.16, 0.57) |

ESSDAI, EULAR Sjögren's Syndrome Disease Activity Index; FC, fold-change; IQR, interquartile range

### Conclusions

CD40-CD40L blockade with Dazodalibep in patients with SS reduces systemic disease activity by inhibiting T and B cell costimulation, as evidenced by treatment related reductions in blood biomarkers downstream of these pathways.

### Example 4-CD40L Blockade with Dazodalibep Significantly Improves Disease Activity, Swollen and Tender Joint Counts, while Reducing Multiple T- and B-Cell Biomarkers in the MIDORA Phase 2 Study in Patients with Moderate-to-Severe Active Rheumatoid Arthritis

### Objective

To assess the impact of dazodalibep on T- and B-cells in MIDORA phase 2 clinical trial of patients with rheumatoid arthritis.

### Methods

This biomarker study focused on a total of 47 adult patients with moderate-to-severe active rheumatoid arthritis (RA) who inadequately responded to methotrexate, other conventional disease-modifying anti-rheumatic drugs, or tumor necrosis factor-α (TNFα) inhibitors. Patients in this biomarker study were randomized to intravenously receive 1500 mg or 3000 mg of dazodalibep or placebo. Disease Activity Score-28 with C-Reactive Protein (DAS28-CRP), Patient Global Assessment (PGA), Simplified Disease Activity Index (SDAI), Clinical Disease Activity Index (CDAI), Physician Global Assessment of Disease Activity (MDGA), Tender Joint Count (TJC), and Swollen Joint Count (SJC) were evaluated. B and T cell subsets such as plasmablasts (CD27^{br}/CD38^{br}/IgD− subsets of CD19+ cells), precursor memory B cells (CD11c^{br} subsets of CD19+ cells), T follicular helper cells (CXCR5+/ICOS+ subsets of CD3+/CD4+ cells), proliferation of post-switch memory B cells (Ki67+ subsets of CD27^{br}/IgD−/CD19+ cells), and proliferation of total T-cells (Ki67+ subsets of CD3+ cells) were immunophenotyped using flow cytometry in whole blood. Serum levels of CXCL13 and rheumatoid factor (RF) autoantibodies were assessed by immunoassay and nephelometry, respectively.

### Results

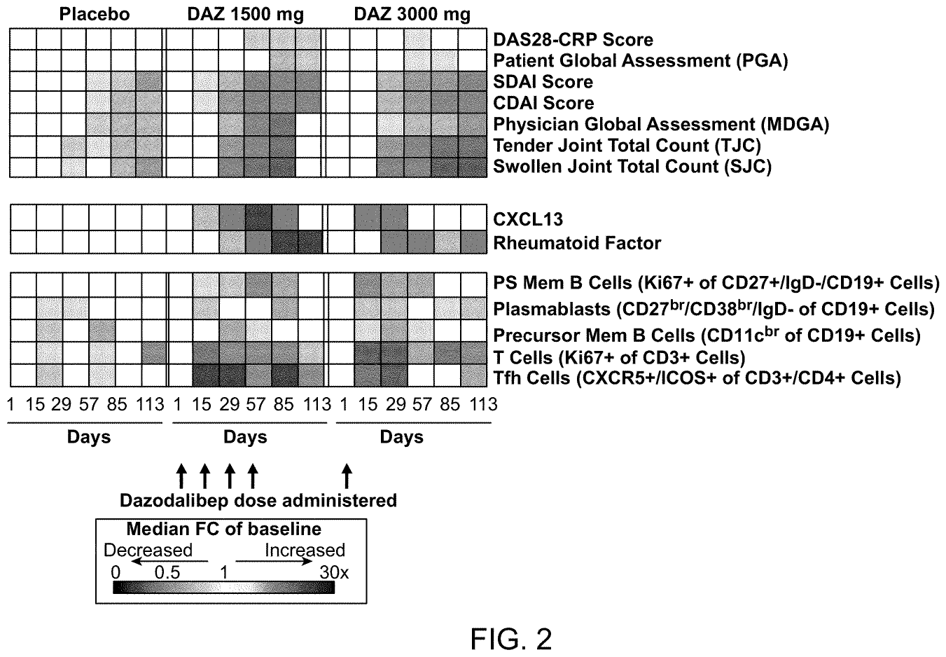

DAZ significantly improved DAS28-CRP, PGA, SDAI, CDAI, and MDGA scores as well as tender and swollen joint total counts in patients with RA (FIG. 2, top). Additionally, DAZ treatment reduced CXCL13 and RF levels in patients with RA as compared to placebo (FIG. 2, middle). Compared to placebo, DAZ treatment also resulted in reduced number of plasmablasts (CD27^{br}/CD38^{br}/IgD− subsets of CD19+ cells), T follicular helper cells (CXCR5+/ICOS+ subsets of CD3+/CD4+ cells), proliferation of post-switch memory B cells (Ki67+ subsets of CD27^{br}/IgD−/CD19+ cells), and proliferation of total T-cells (Ki67+ subsets of CD3+ cells), as well as decreases in serum levels of CXCL13 and RF, with the exception of precursor memory B cells (CD11c^{br} subsets of CD19+ cells) (FIG. 2, bottom).

### Conclusions

DAZ-mediated CD40L blockade in patients with moderate-to-severe active RA demonstrated significant improvements in disease activity scores such as DAS28-CRP, Patient Global Assessment, SDAI, CDAI, and Physician Global Assessment including tender and swollen joint counts. Additionally, DAZ reduced multiple CD40L-CD40 downstream biomarkers by disrupting T- and B-cell costimulation, downstream biomarkers of GC formation, activity, and autoantibody production. Together, these data demonstrated the breadth of dazodalibep impact on patients with rheumatoid arthritis and supports clinical efficacy findings.

### Example 5—Biomarker Assessment

A study was conducted that evaluated the efficacy of Dazodalibep when administered to subjects with Sjögren's syndrome (SS). The study focused on the markers downstream of CD40L-mediated T and B cell costimulation: CXCL13, Ki67 (proliferation), CD11c^{br}, CD27^{br}CD38^{br}, SS-A, SS-B, RF, and ANA.

### Results

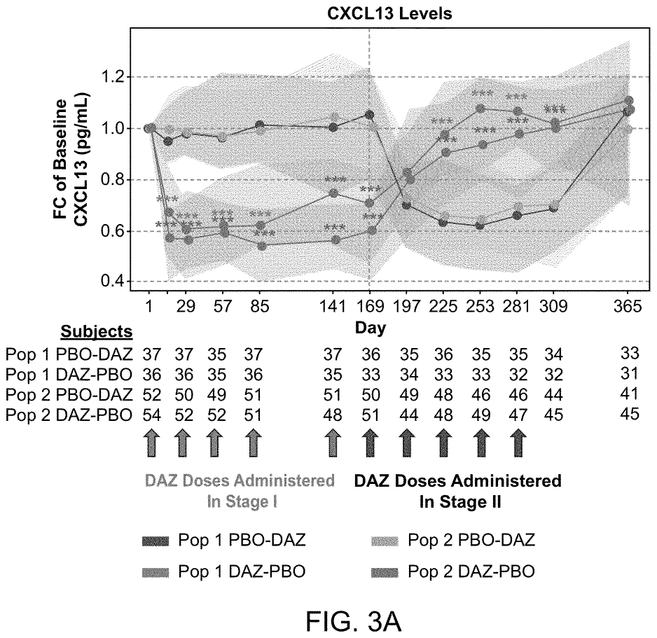

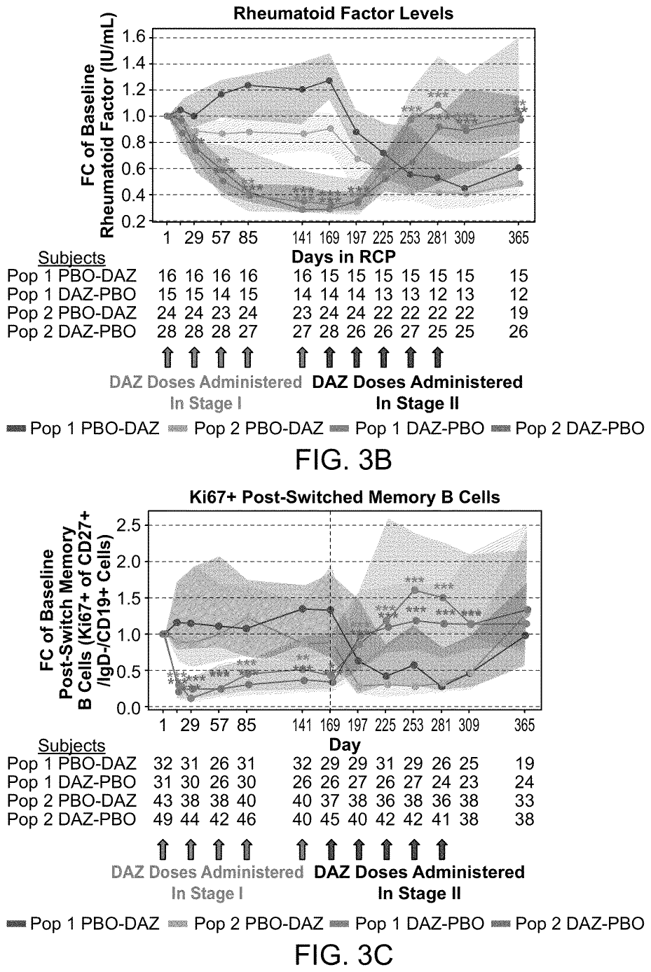

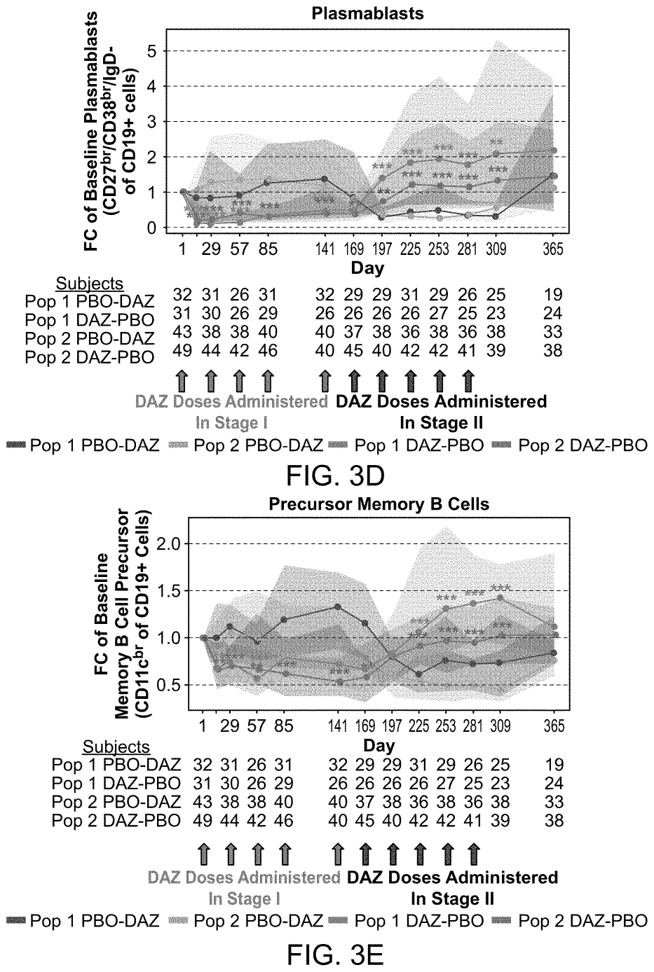

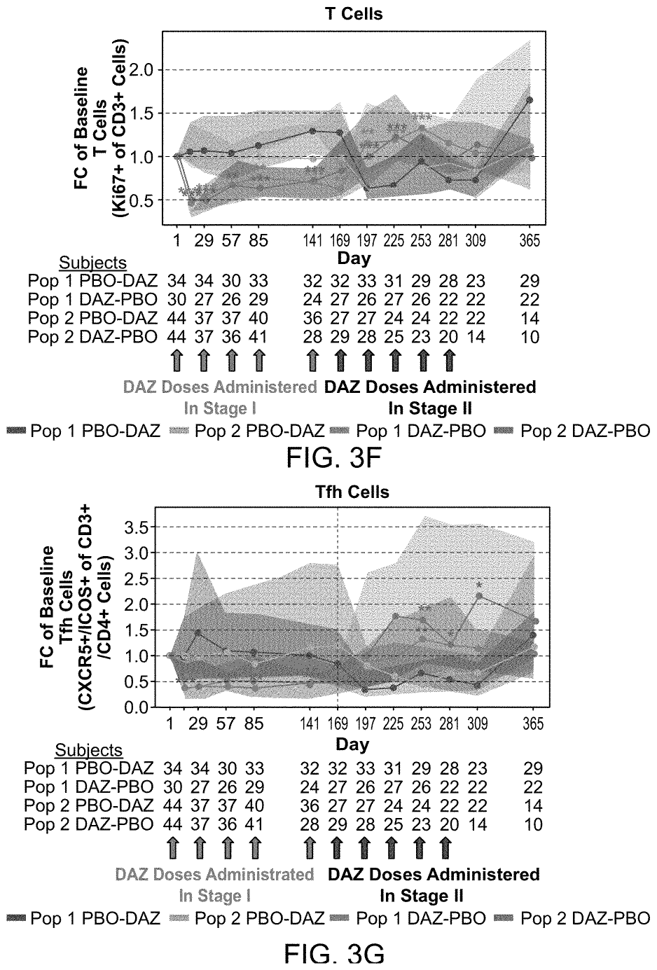

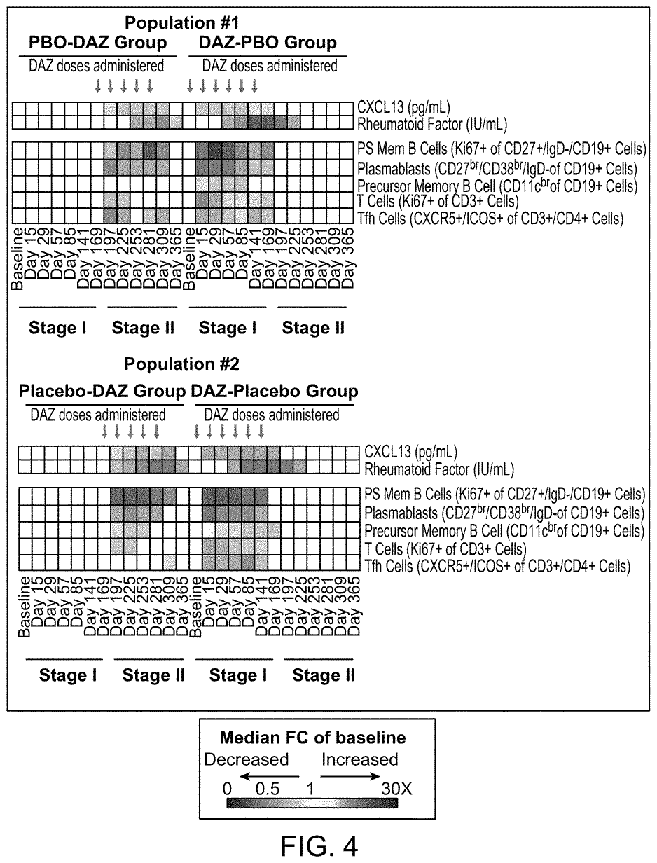

Results show that subjects dosed with Dazodalibep significantly and rapidly reduced multiple pathogenic biomarkers of CD40L mediated B and T cell pathway costimulation and proliferation; and downstream biomarkers of germinal center formation, activity, and autoantibody production. As shown in FIGS. 3A-3G and FIG. 4, many of these biomarker reductions occurred as early as Day 15 and were sustained until Day 169 (primary endpoint). Indeed, treatment with Dazodalibep allows for blocking CD40L from binding to its cognate receptor, CD40, in patients with Sjögren's across populations with either moderate-to-severe systemic disease activity or high symptomatic burden.

### Example 6. Dazodalibep (Anti-CD40L) Effectively Reduces Multiple Proteins Associated with B-, T-, and Dendritic Cell Biomarkers and Corroborates Immunophenotyping Findings from the Phase 2 Study in Sjögren's Populations with Either Moderate-to-Severe Disease Activity or High Symptomatic Burden

### Objective

To evaluate the effects of dazodalibep on the serum proteome in phase 2 clinical trial of patients with Sjögren's disease.

### Method

This study enrolled two distinct populations with Sjögren's disease. Population 1 (Pop1) included 74 patients with moderate-to-severe systemic disease activity as defined by the EULAR Sjögren's Syndrome Disease Activity Index (ESSDAI)≥5. Population 2 (Pop2) consisted of 109 patients with unacceptable symptom burden and limited extraglandular systemic involvement as defined by the EULAR Sjögren's Syndrome Patient Reported Index (ESSPRI)≥5 and ESSDAI<5. Eligible participants were randomized 1:1 to intravenously receive DAZ 1500 mg or placebo. Olink® Explore 3072 panel was used to comprehensively profile the serum proteome at baseline, Day 15, and Day 169. Protein profiles were analyzed using AUC-ROC analysis to identify proteins which differentiated patients with Sjögren's disease at baseline from age-matched healthy individuals (n=50). A mixed linear model was applied to test for proteins impacted by dazodalibep treatment. Matrix decomposition techniques and pathways analysis were applied to identify protein networks within the dataset. Twenty-nine protein biomarkers assessed included: TNFRSF13B, BAFF, CXCL10, CXCL12, CXCL13, IL6, CCL21, CXCL9, IL12B, TNFRSF4, LAMP3, CD22, CD79B, SLAMF7, IL10, TNFRSF8, CXCL11, CCL19, TNFRSF9, TNFRSF6B, NOS3, CD72, ADA2, TL2RA, CD27, PDCD1, IL12A, IL12B, LAG3, ADGRE1, TIGIT, FCAMR, and CD80.

### Results

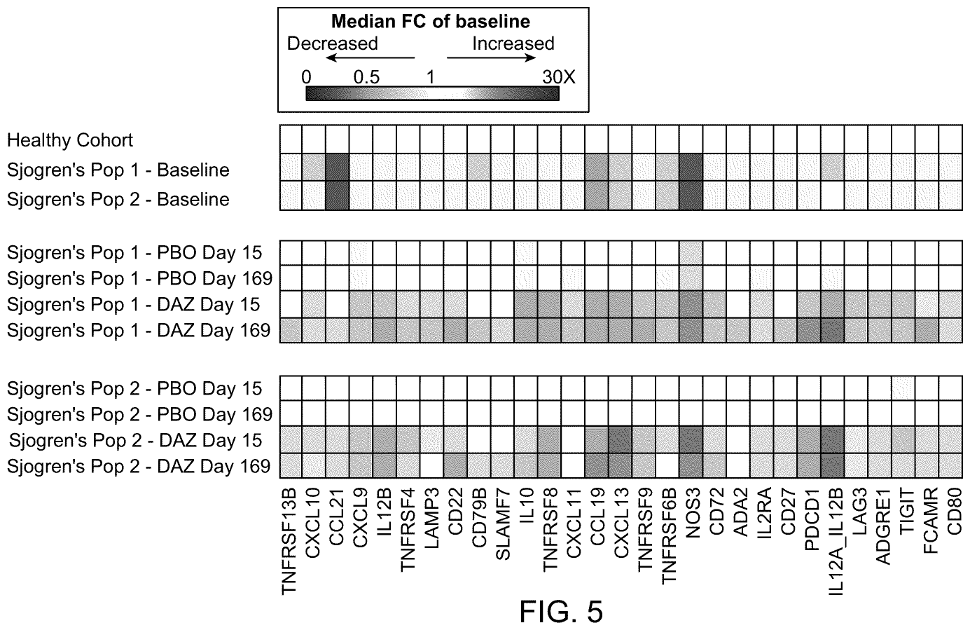

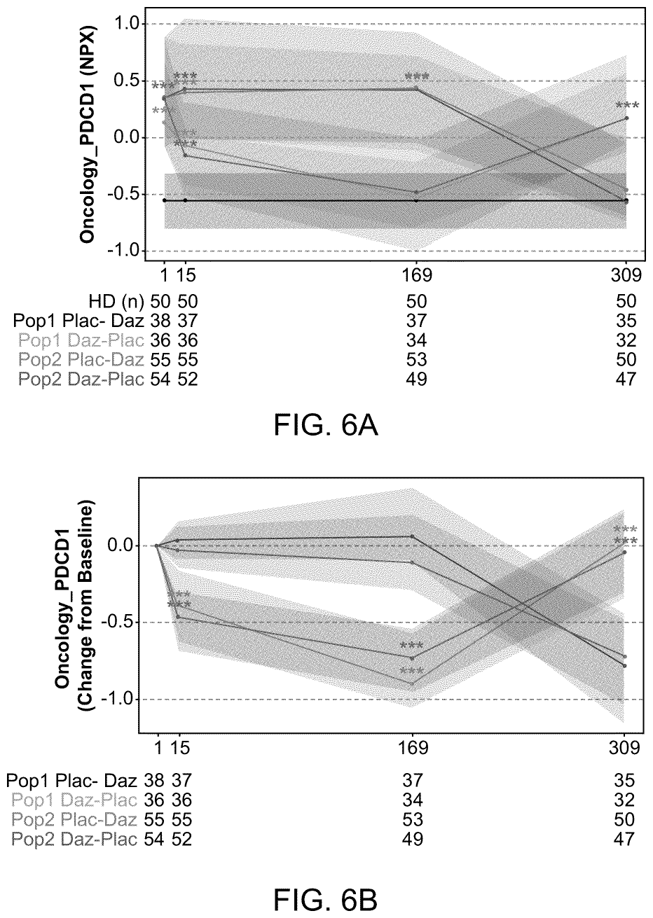

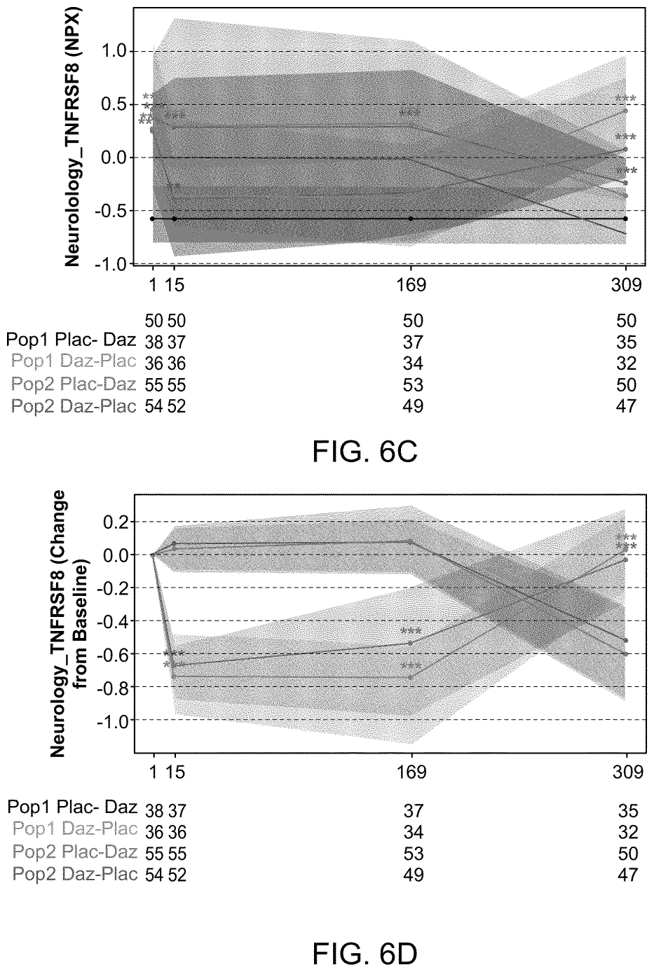

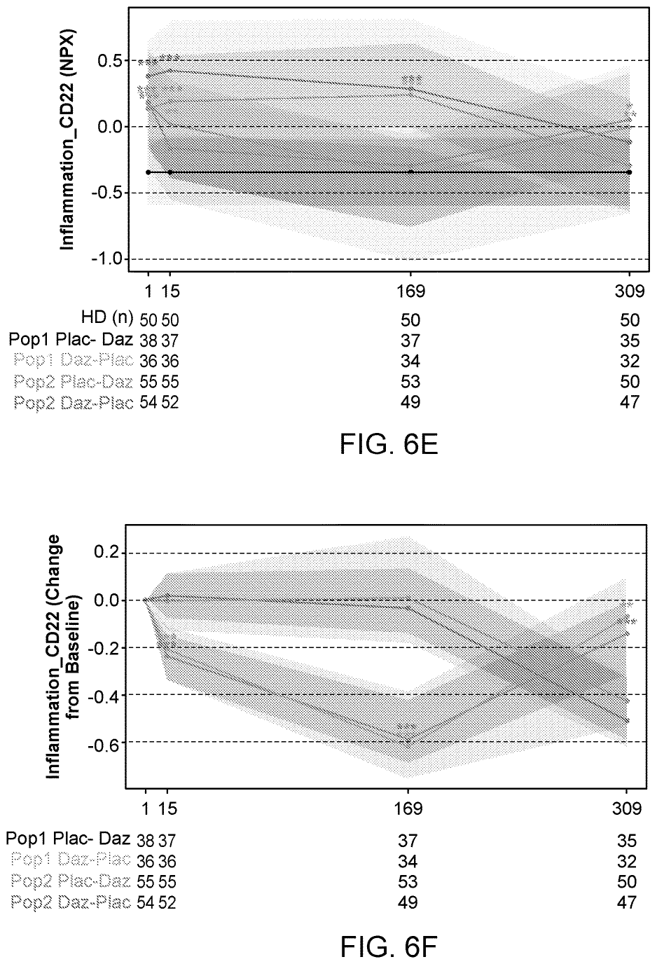

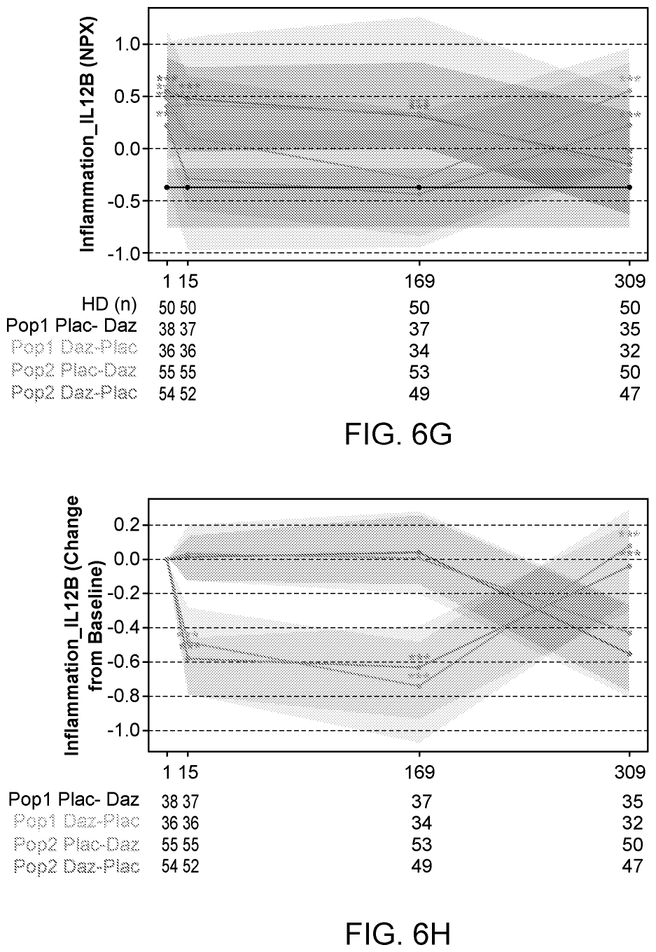

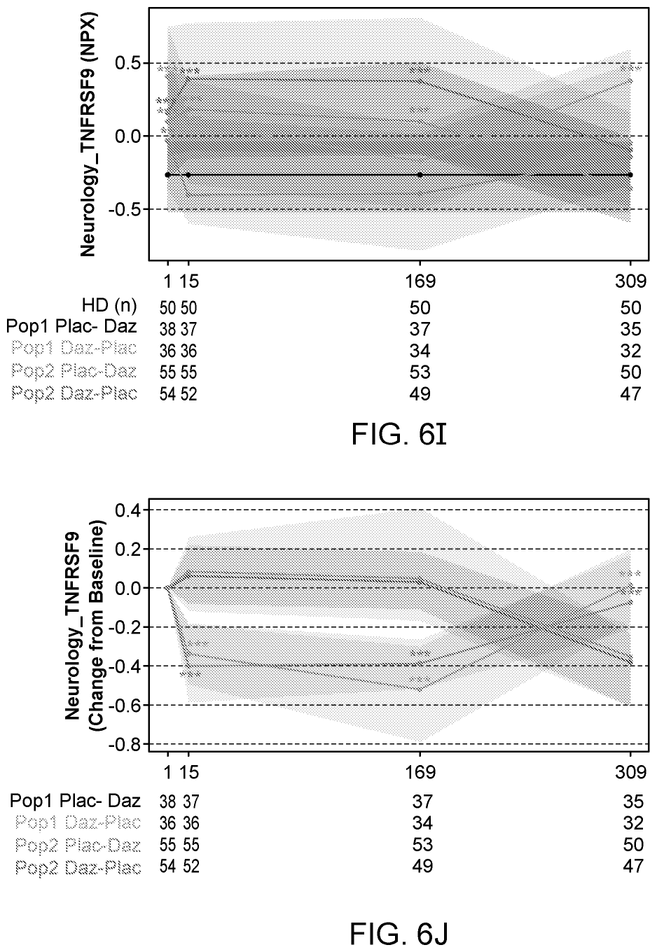

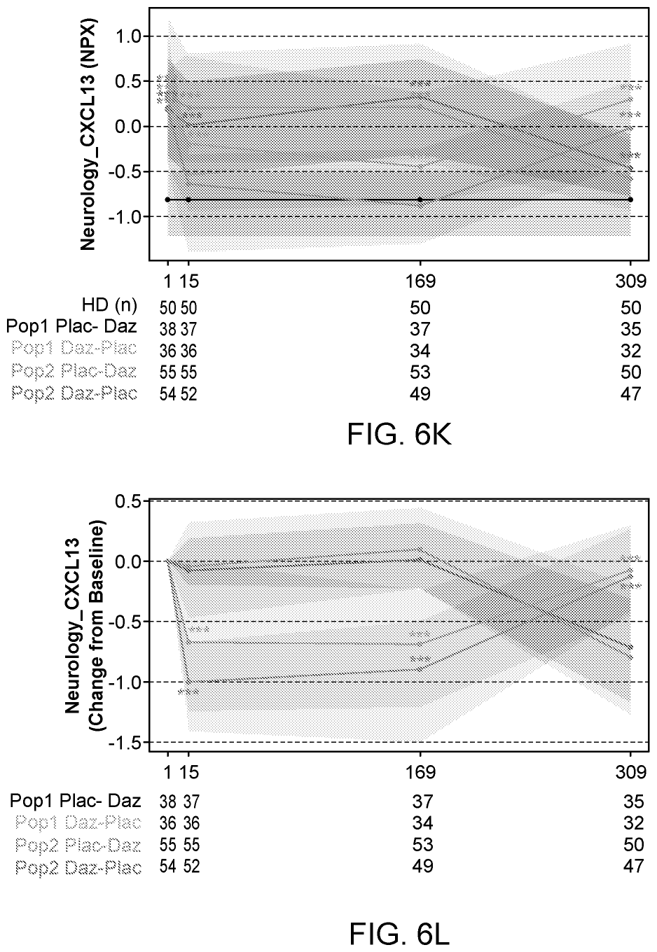

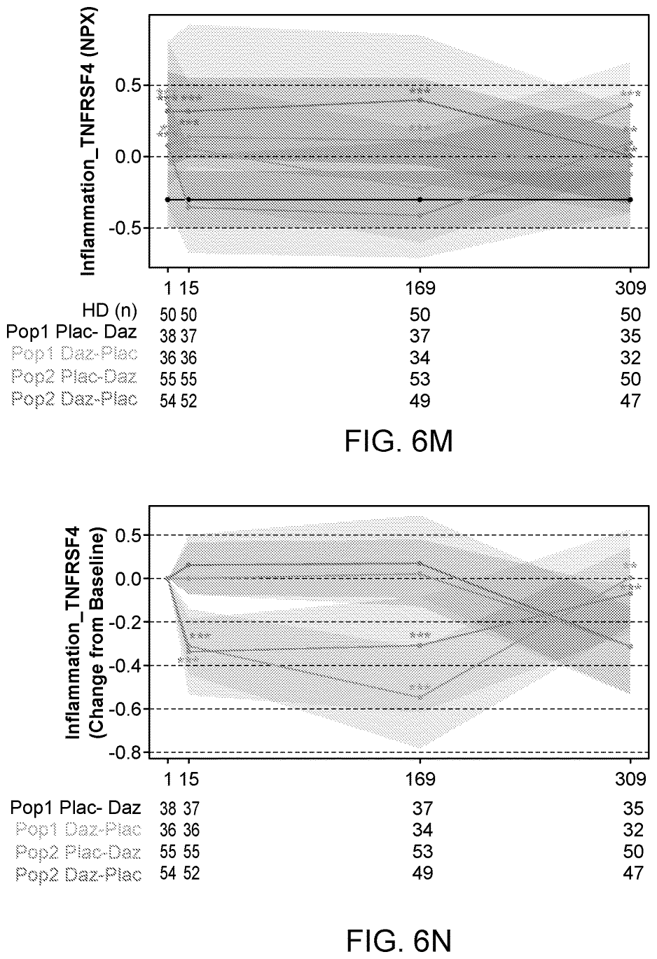

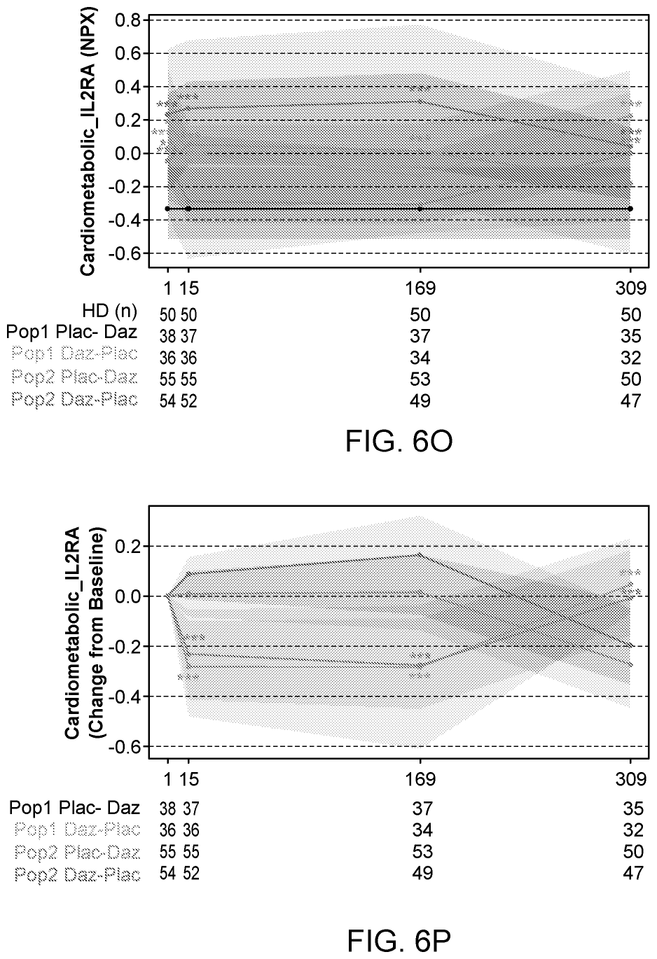

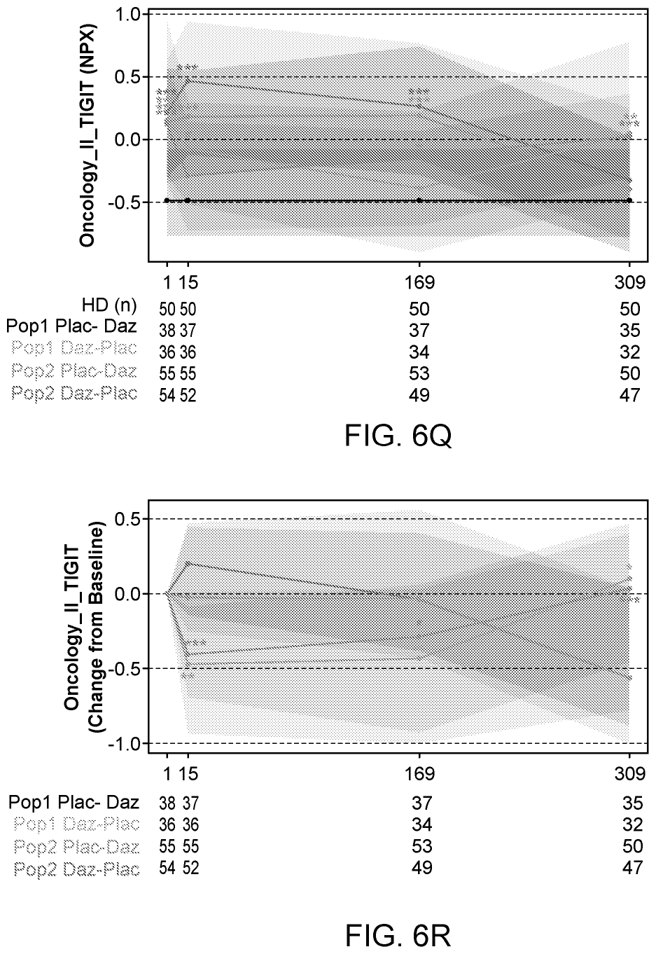

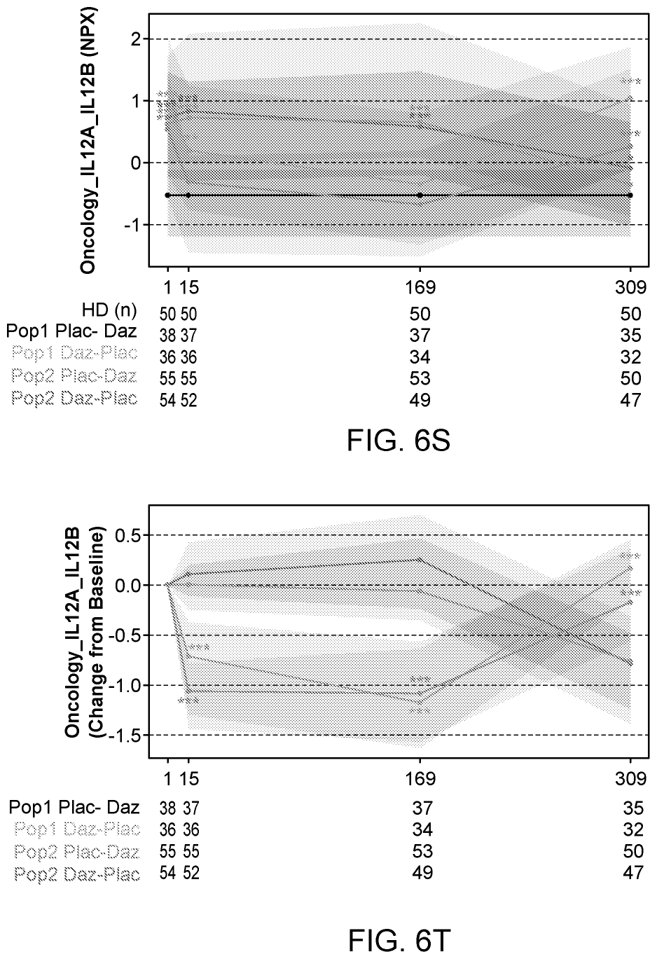

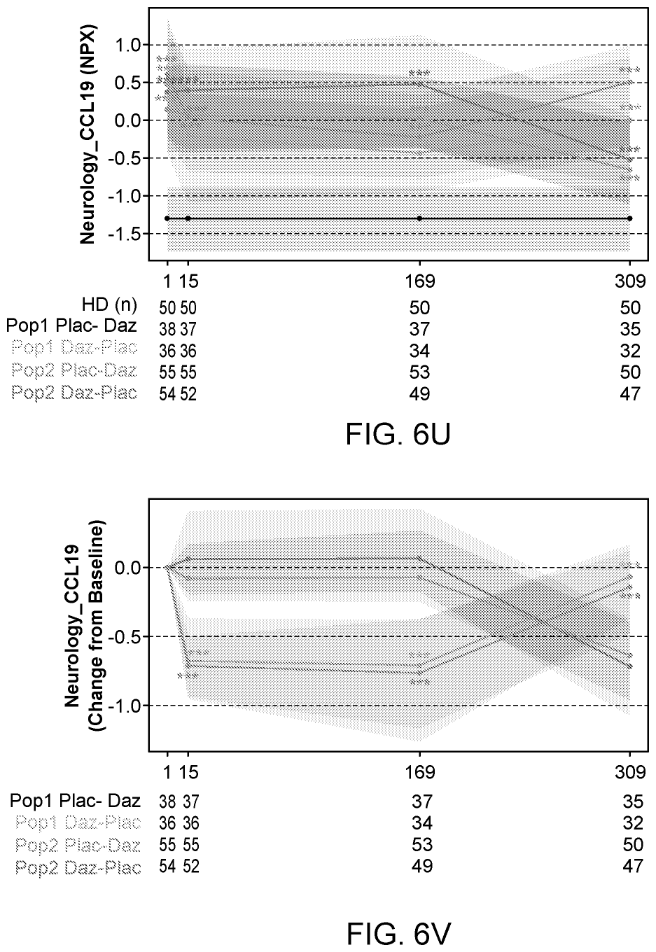

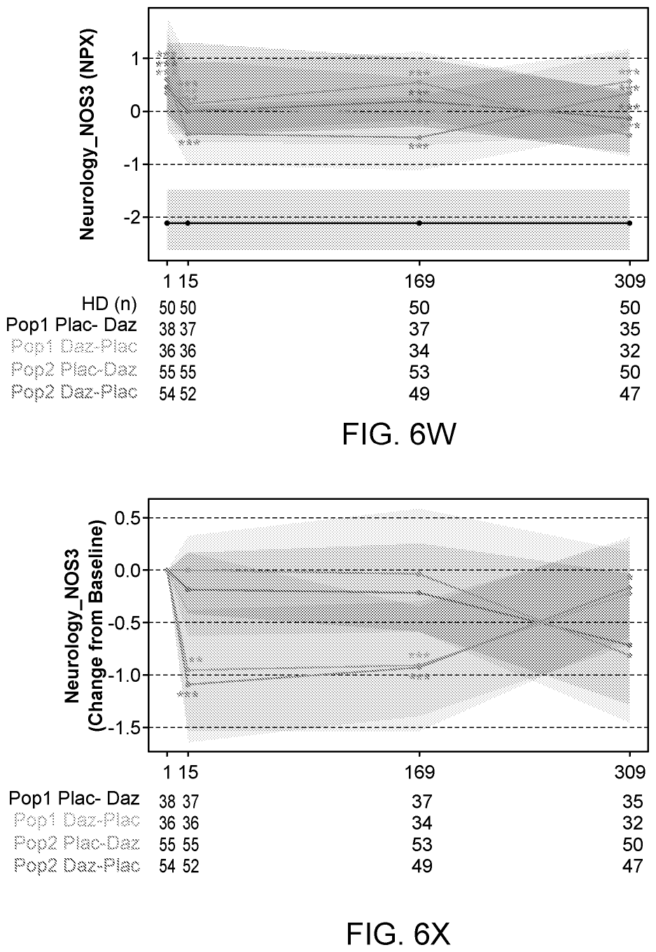

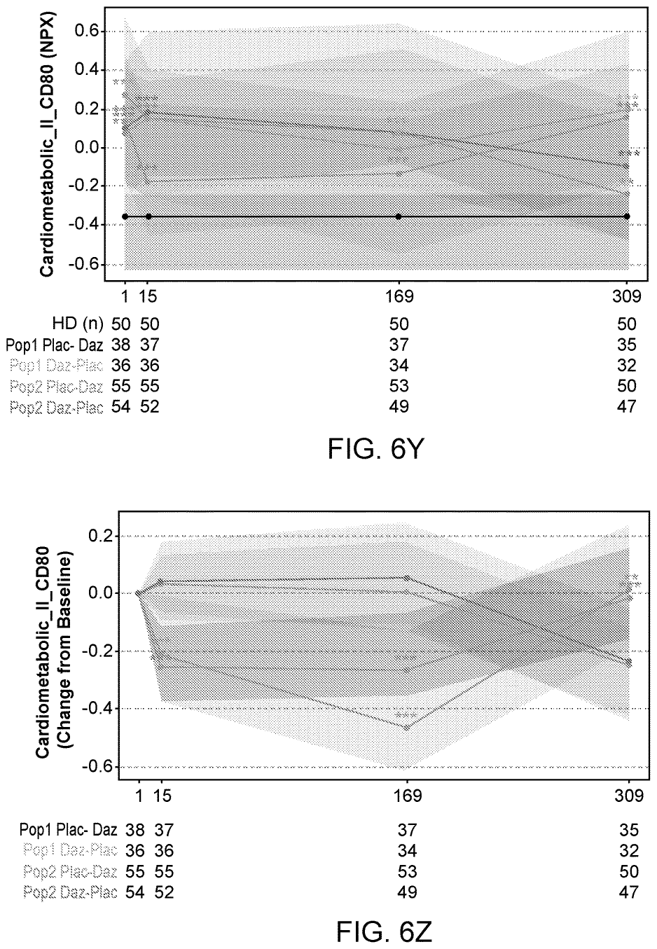

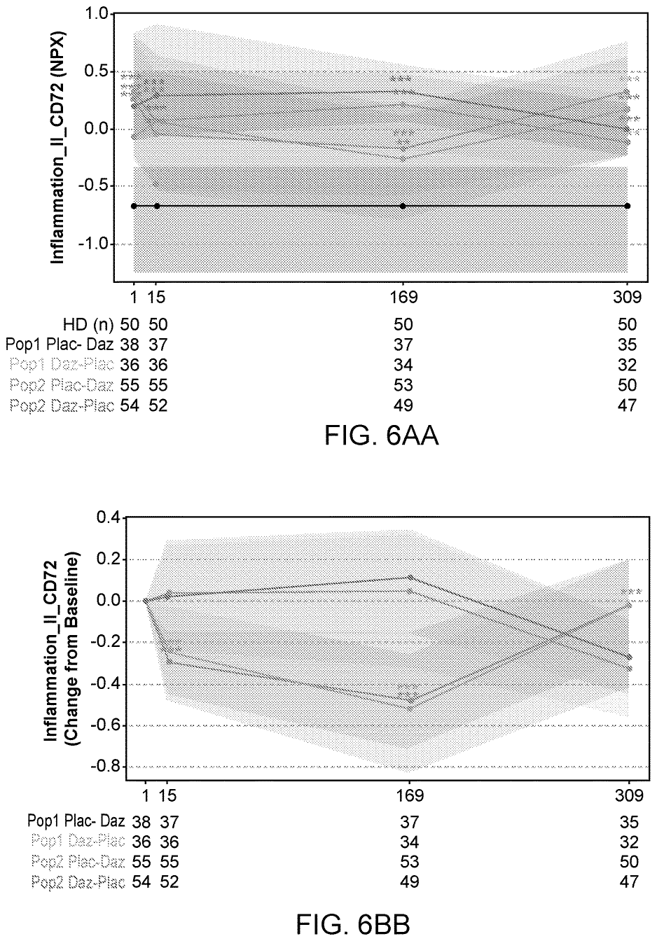

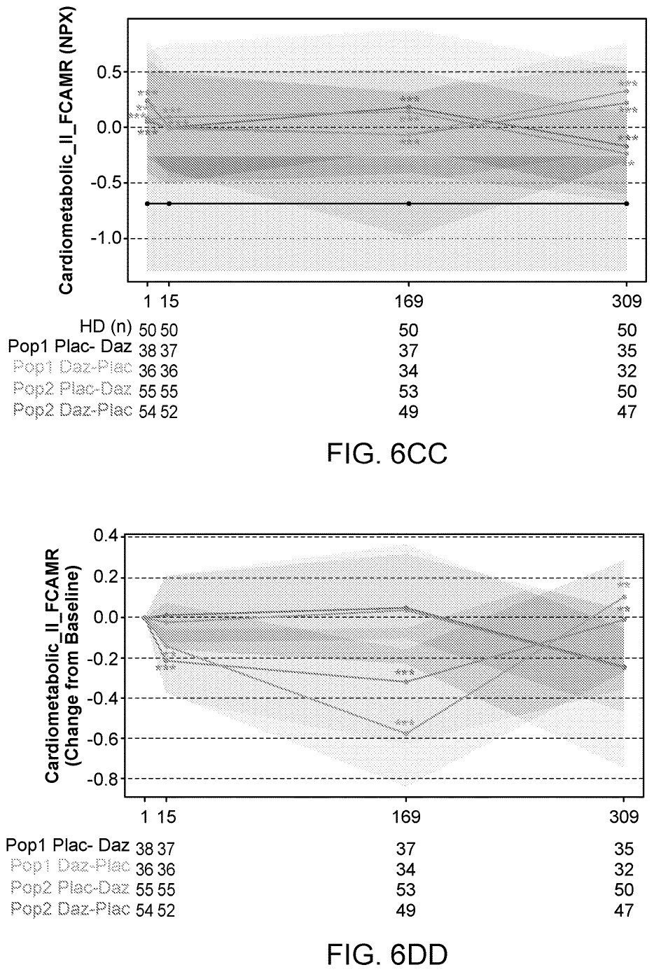

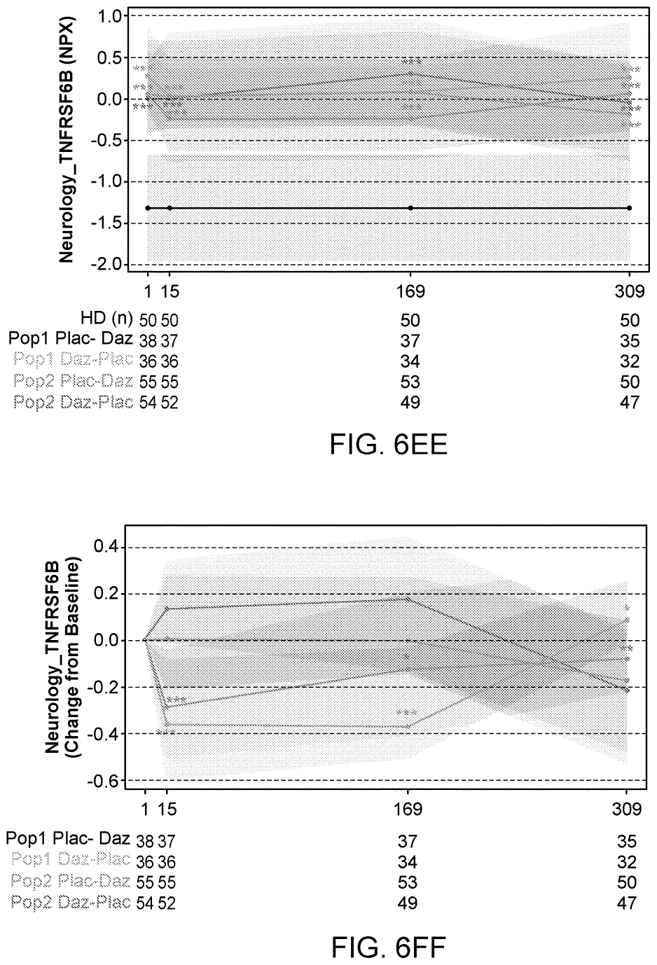

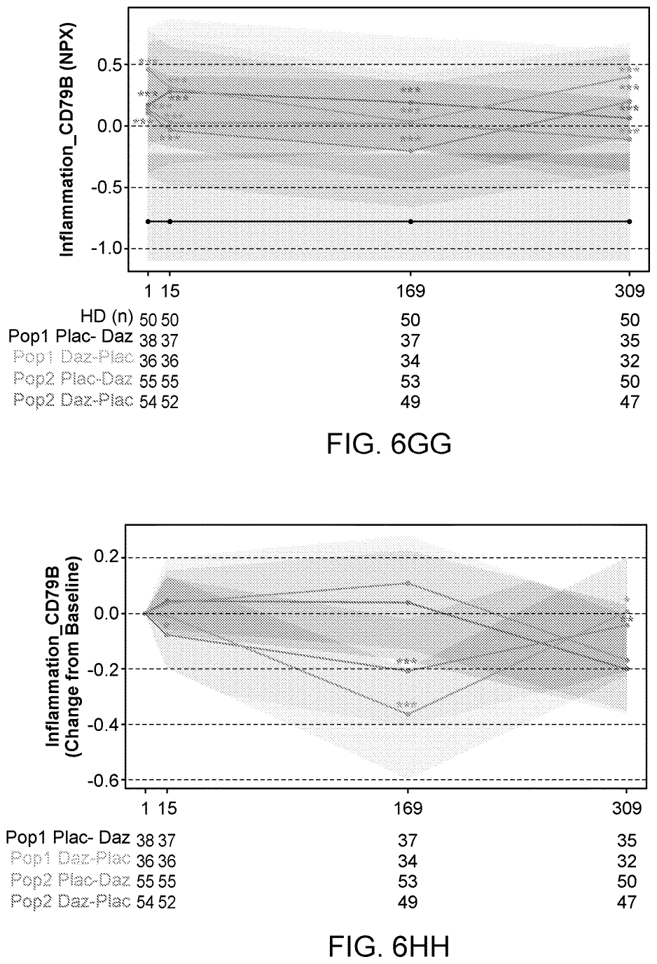

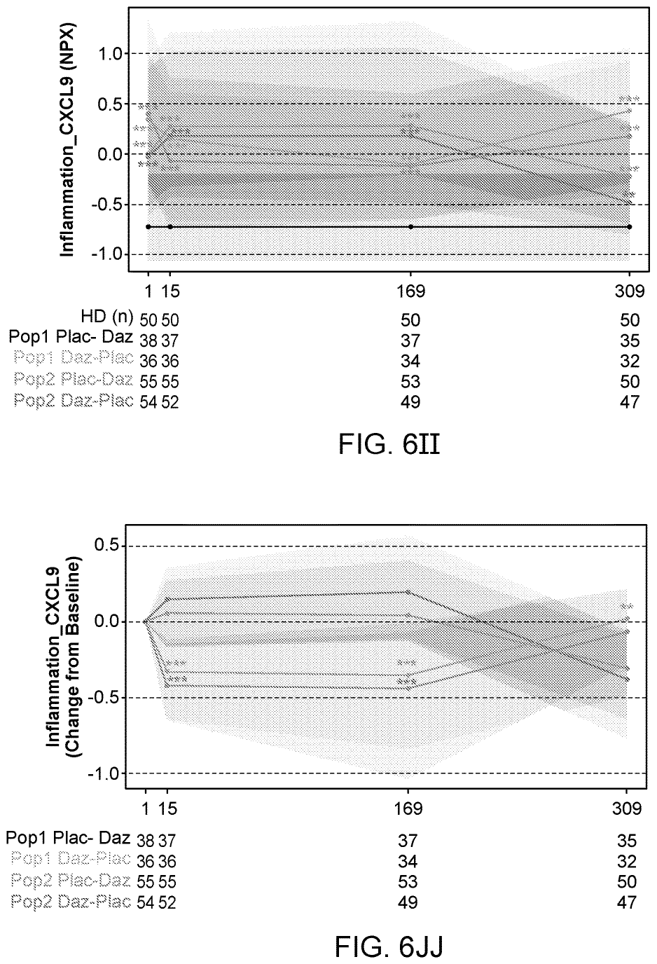

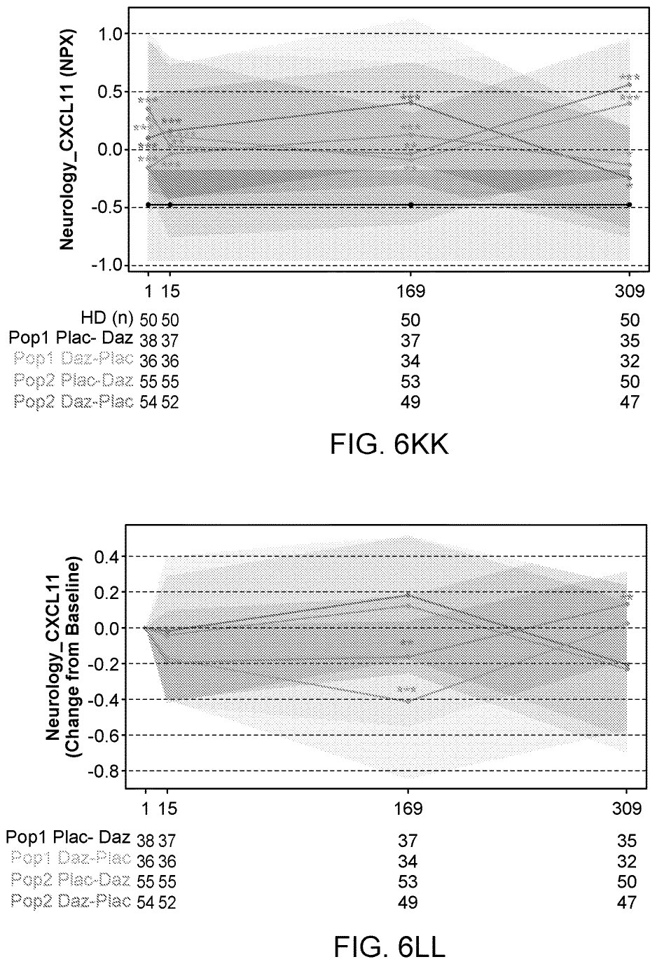

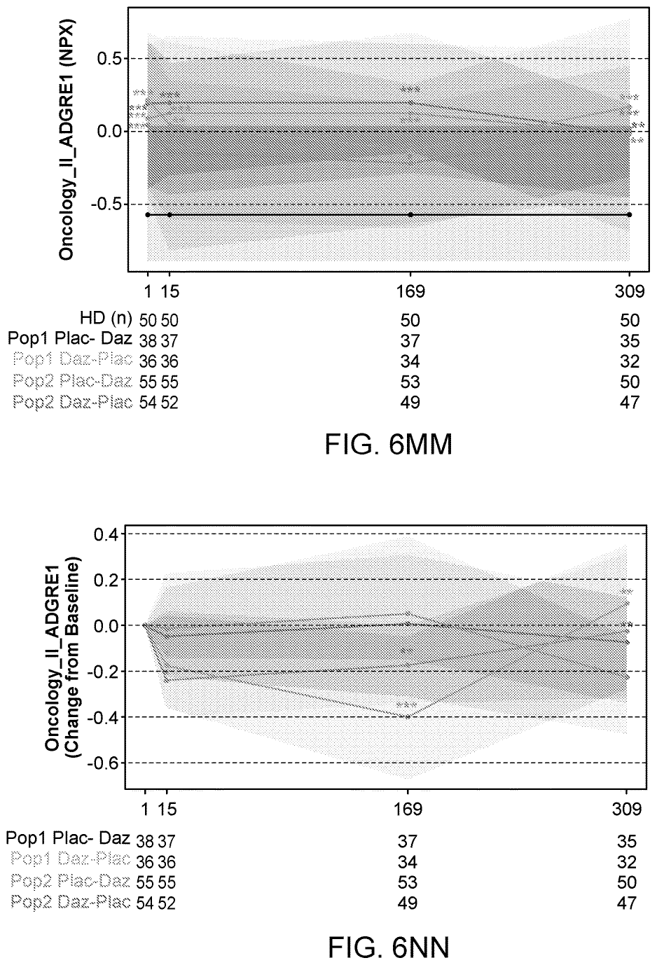

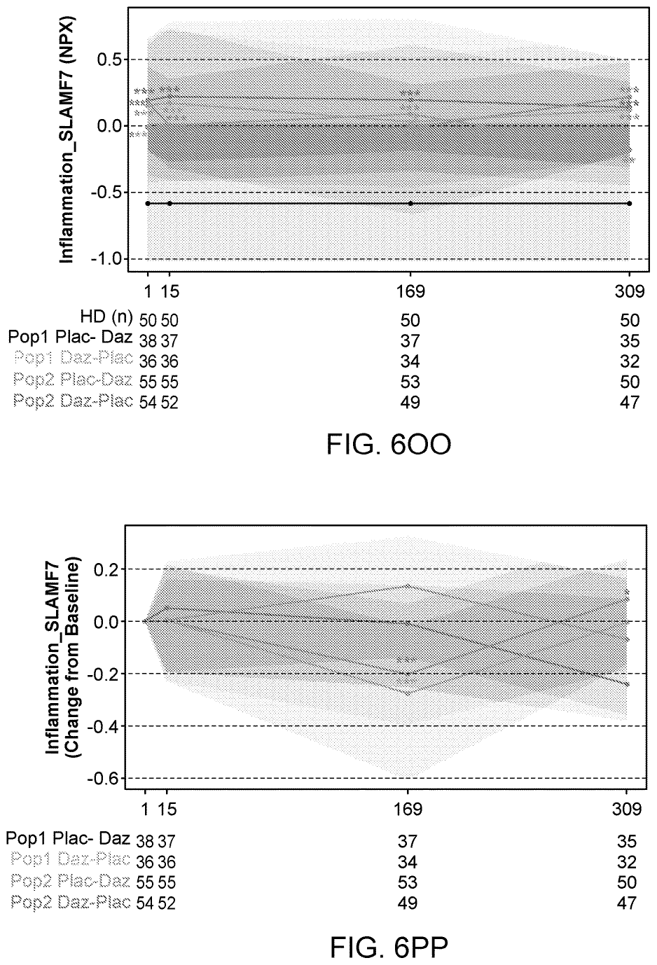

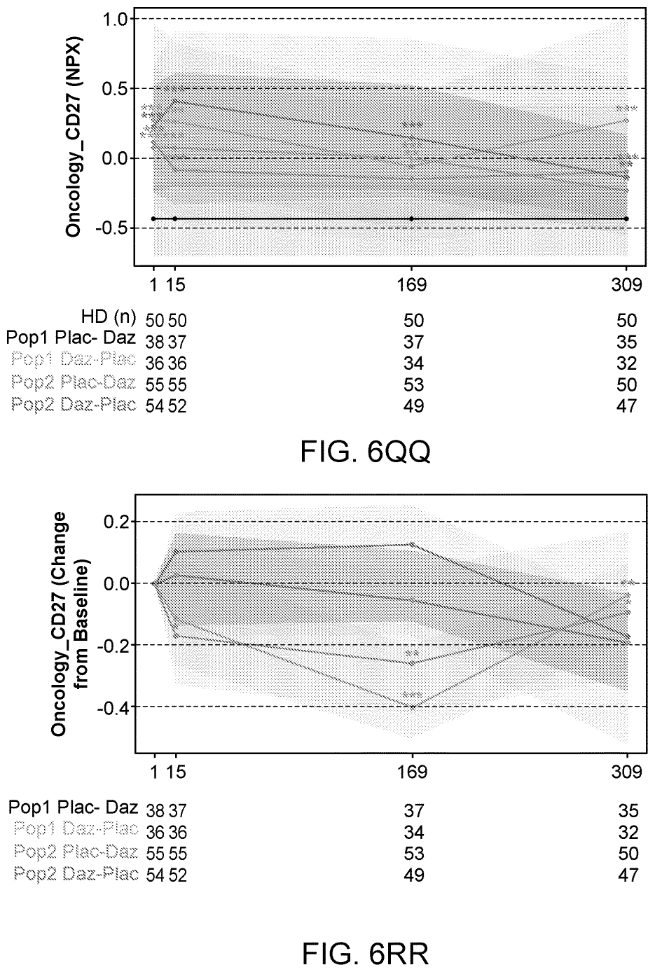

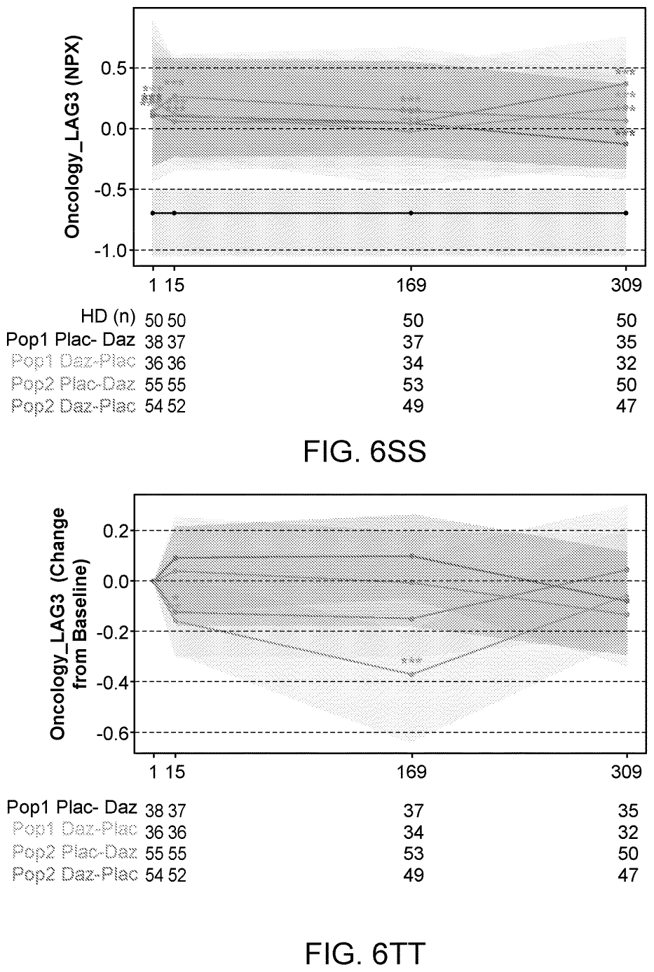

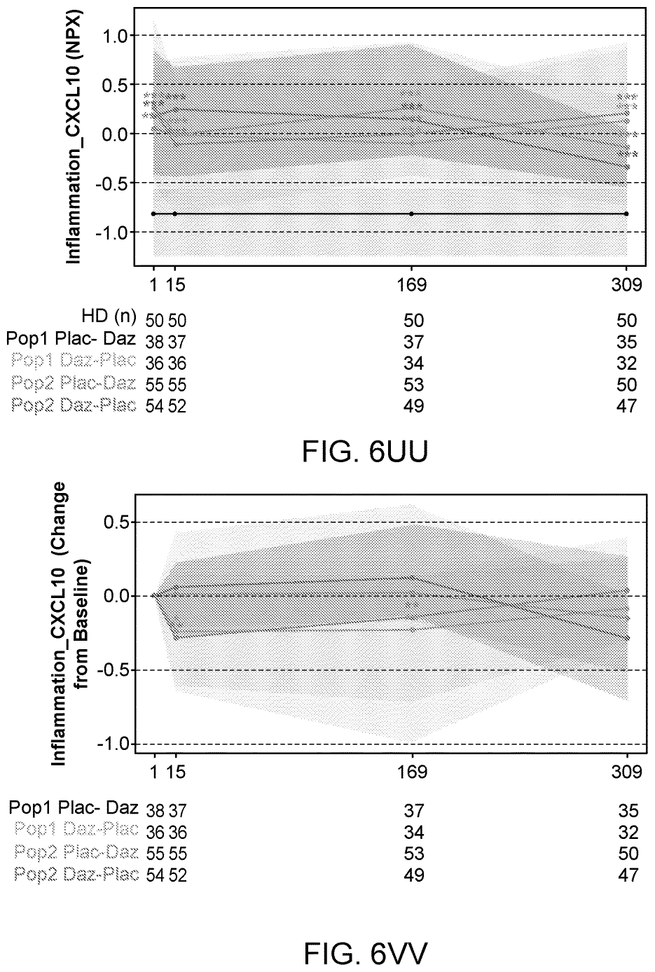

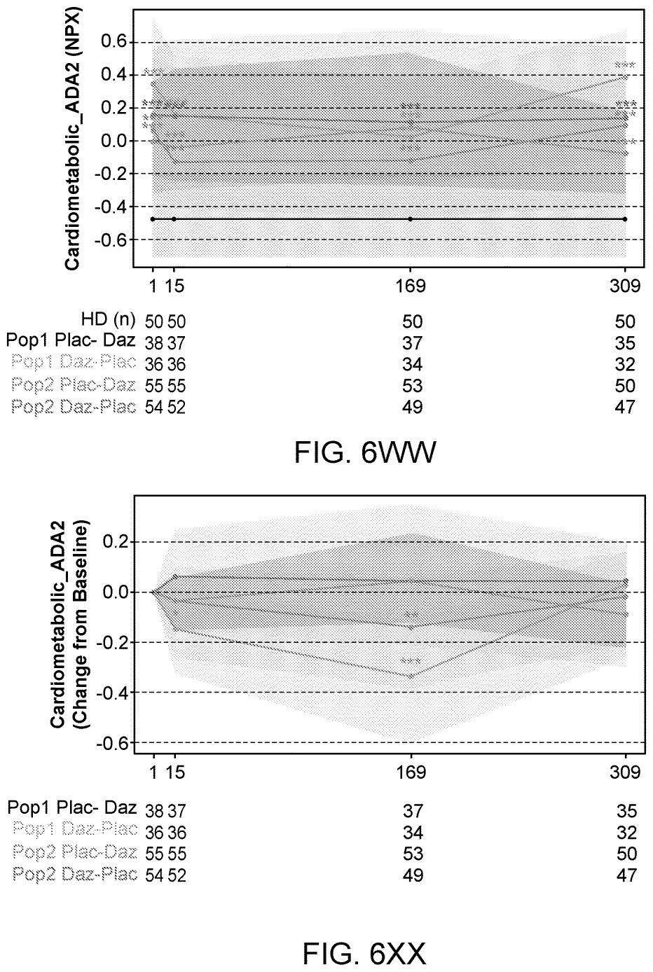

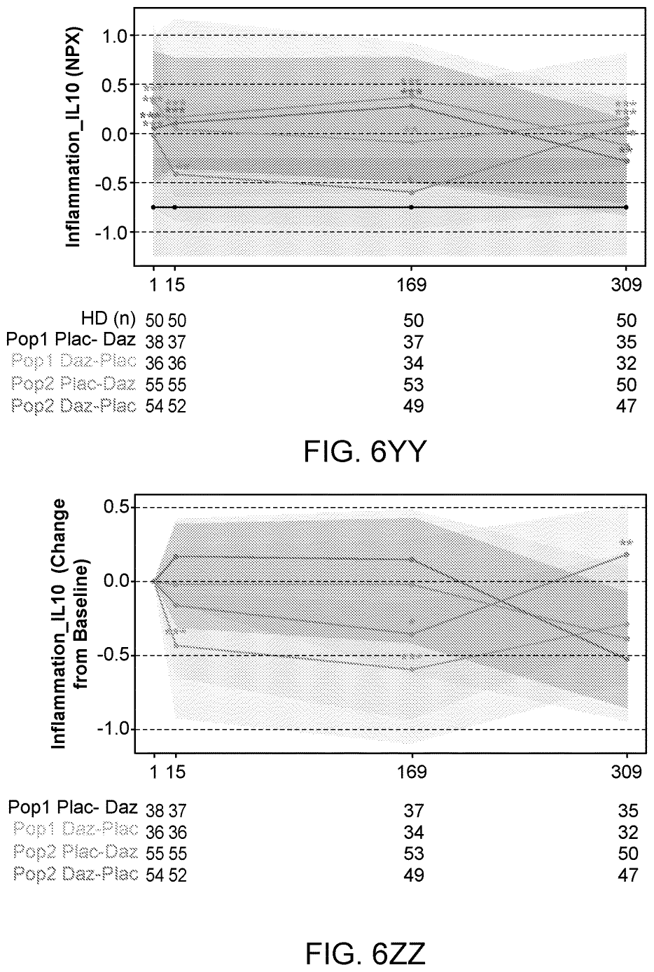

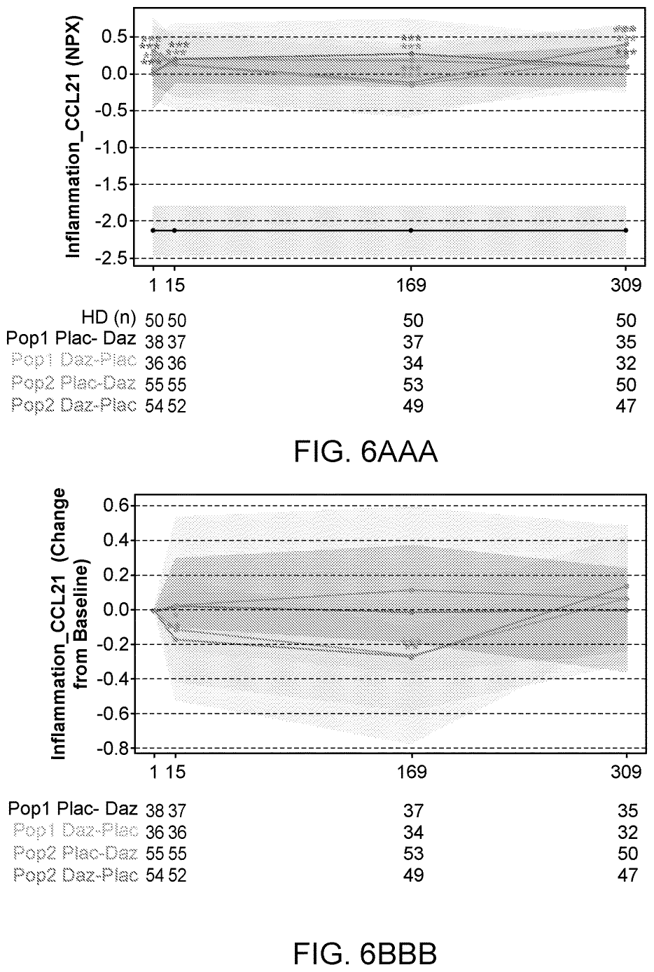

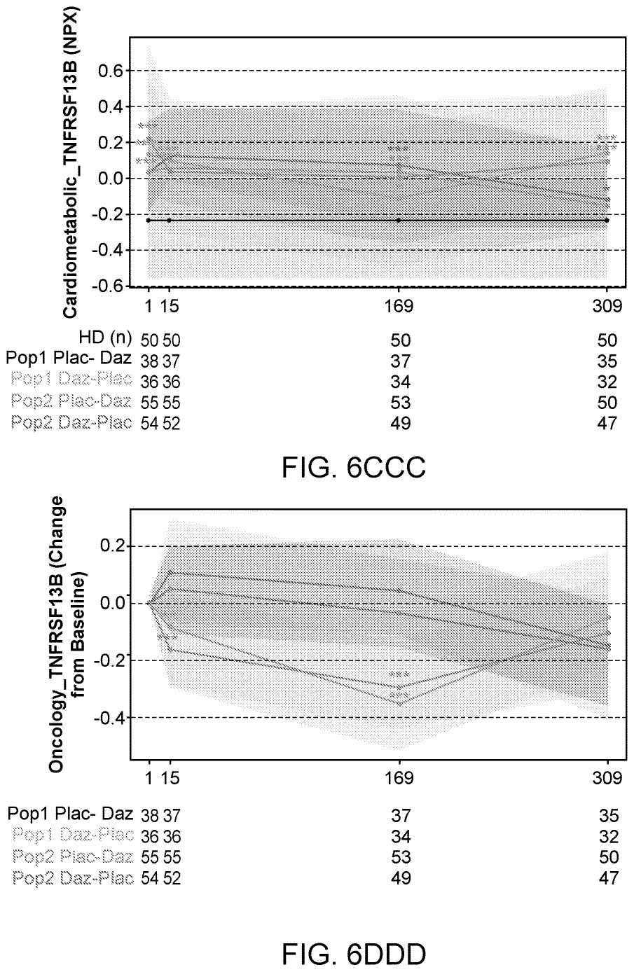

Although unsupervised machine learning identified a core set of 29 proteins elevated in patients with Sjögren's disease at baseline compared to healthy individuals, there were no significant differences in these biomarkers between Pop 1 and Pop 2 at baseline. Pathway assessments revealed these 29 biomarkers were enriched for activation of B-, T-, and dendritic cells (e.g., LAMP3) and were significantly reduced by DAZ treatment in both Sjögren's populations 1 and 2 (FIG. 5). Protein profiles were analyzed using AUC-ROC analysis to identify proteins which differentiated subjects with Sjögren's disease at baseline from healthy individuals (FIG. 5, FIGS. 6A-6DDD). For all 29 biomarkers, a Benjamini Hochberg False Discovery Rate (FDR)<10% was implemented to determine change from baseline significance. Factor Analysis followed by a Varimax rotation was further used to identify proteins that shared correlation with high loadings (>0.45) across a common vector. Nine biomarkers (PDCD11, TNFRSF8, CD22, IL12B, TNFRSF9, CXCL13, TNFRSF4, IL2RA, and TIGIT) fell near or within the expression level of a healthy subject by Day 169 (FIGS. 6A-6R).

### Conclusion

Compared to a healthy cohort, serum proteomics profiling revealed a core set of biomarkers elevated in patients with Sjögren's disease at baseline and were associated with inflammatory and adaptive immune responses, particularly activation of B-, T-, and dendritic cells and were significantly reduced (FDR≤10%) in Pop1 and Pop2 subjects with Sjögren's disease by Dazodalibep treatment. Importantly, these biomarkers were shown to be significantly reduced by dazodalibep.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as an acknowledgment or any form of suggestion that they constitute valid prior art or form part of the common general knowledge in any country in the world.

1. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the subject in need has increased B cells of subsets selected from the group consisting of: Ki67+CD27+ memory cells, CD27 high CD38 high plasmablasts, and CD11c high atypical memory cells, as compared to a baseline level of the subject or a level in an untreated subject.

2. The method of claim 1, wherein at least one CD40L-specific monomer subunit comprises the seven beta strands designated A, B, C, D, E, F, and G, wherein the beta strand A comprises SEQ ID NO: 5, the beta strand B comprises SEQ ID NO: 6, the beta strand C comprises SEQ ID NO: 17, the beta strand D comprises SEQ ID NO: 18, the beta strand E comprises SEQ ID NO: 19, the beta strand F comprises SEQ ID NO: 20, the beta strand G comprises SEQ ID NO: 21.

3. The method of claims 1-2, wherein the subject has a European League Against Rheumatism (EULAR) Sjögren's Syndrome Disease Activity Index (ESSDAI) score≥5.

4. The method of claim 3, wherein the ESSDAI score is assessed based on the ESSDAI domains consisting of cutaneous, renal, articular, muscular, hematological, glandular, constitutional, lymphadenopathic, and biological.

5. The method of claims 1-2, wherein the subject has
(a) a ESSDAI score≤5,
(b) a EULAR Sjögren's Syndrome Patient Reported Index (ESSPRI) score≥5, and
(c) whole stimulated salivary flow>0.1 mL/min.

6. The method of any one of claims 1-5, wherein the Tn3 scaffold is administered as an induction dose and as a maintenance dose thereafter.

7. The method of claim 6, wherein the induction dose comprises administering the Tn3 scaffold once about every 2 weeks for at least 3 doses.

8. The method of claim 6, wherein the maintenance dose comprises administering the Tn3 scaffold once about every 4 weeks for at least 4 doses.

9. The method of claim 8, wherein the time between the last induction dose and the first maintenance dose is about 4 weeks.

10. The method of any one of claims 1-5, wherein the Tn3 scaffold is administered once about every 4 weeks, once about every 2 months, once about every 3 months, once about every 4 months, or once about every 6 months.

11. The method of claim 10, wherein the Tn3 scaffold is administered for at least 4 doses.

12. The method of claim 11, wherein the Tn3 scaffold is administered for at least 5 doses.

13. The method of any one of claims 1-12, wherein the Tn3 scaffold is administered intravenously, subcutaneously, orally, intramuscularly, intrathecally, sublingually, rectally, vaginally, cutaneously, systemically, topically, transdermally, or by way of inhalation.

14. The method of claim 13, wherein the Tn3 scaffold is administered intravenously.

15. The method of any one of claims 1-14, wherein the Tn3 scaffold comprises two CD40L-specific monomer subunits connected in tandem.

16. The method of claim 15, wherein the two CD40L-specific monomer subunits each comprise SEQ ID NO: 3.

17. The method of any of one of claims 1-16, wherein the CD40L-specific monomer subunits are connected by a linker.

18. The method of any one of claims 1-17, wherein at least one CD40L-specific monomer subunit is fused or conjugated to a polyethylene glycol (PEG) directly.

19. The method of any one of claims 1-17 wherein at least one CD40L-specific monomer subunit is fused or conjugated to a polyethylene glycol (PEG) via a linker.

20. The method of claim 17, wherein the linker comprises a peptide linker.

21. The method of claim 20, wherein the linker comprises SEQ ID NO: 7, SEQ ID NO: 8, SEQ ID NO: 9, or SEQ ID NO: 10.

22. The method of claim 21, wherein at least one CD40L-specific monomer subunit is fused or conjugated to an albumin.

23. The method of claim 22, wherein the albumin is human serum albumin (HSA).

24. The method of claim 23, wherein the HSA is a variant HSA comprising SEQ ID NO: 4.

25. The method of any one of claims 1-24, wherein the Tn3 scaffold comprising the CD40L-specific monomer subunit is administered at a dose of about 1500 mg, about 2500 mg, or about 3000 mg.

26. The method of claim 25, wherein the dose is about 1500 mg.

27. The method of claim 25, wherein the dose is about 2500 mg.

28. The method of claim 25, wherein the dose is about 3000 mg.

29. A method of treating Sjögren's syndrome (SS) in a subject in need thereof comprising: administering a Tn3 scaffold at a dose of about 1500 mg, about 2500 mg, or about 3000 mg to the subject, wherein the Tn3 scaffold comprises SEQ ID NO: 1, and wherein the subject in need has increased B cells of subsets selected from the group consisting of: Ki67+CD27+ memory cells, CD27 high CD38 high plasmablasts, and CD11c high atypical memory cells, as compared to an otherwise healthy subject.

30. The method of claim 29, wherein the Tn3 scaffold is administered as an induction dose and as one or more maintenance doses thereafter.

31. The method of claim 30, wherein the induction dose and the maintenance doses are the same amount.

32. The method of claim 30, wherein the induction dose and the maintenance doses are different amounts.

33. The method of any one of claims 29-32, wherein at least one dose is 3000 mg.

34. The method of any one of claims 29-33, wherein the induction dose and at least one maintenance dose are administered about 1 month apart, about 2 months apart, about 3 months apart, about 4 months, or about 6 months apart.

35. The method of any one of claims 29-34, wherein the induction dose comprises administering the Tn3 scaffold about every 2 weeks for at least 3 doses and the maintenance dose comprises administering the Tn3 scaffold once about every 4 weeks for at least 4 doses.

36. The method of any one of claims 1-35, wherein the Tn3 scaffold comprises SEQ ID NO: 1.

37. The method of any one of claims 1-36, wherein the administration is effective in reducing a ESSDAI score as compared to an otherwise comparable subject receiving an administration of placebo control.

38. The method of claim 37, wherein the reduction is of at least about 1 point, 2 points, 3 points, 4 points, or 5 points.

39. The method of claim 38, wherein the reduction is of at least about 6 points.

40. The method of any one of claims 1-39, wherein the administration is effective in reducing levels of B cells as compared to the baseline level of the subject or the level in the untreated subject.

41. The method of any one of claims 1-40, wherein the administration is effective in reducing a level of serum CXCL13 as compared to a baseline level in the subject or a level in an untreated subject.

42. The method of any one of claims 1-41, wherein the administration is effective in reducing rheumatoid factor (RF) autoantibodies as compared to a baseline level in the subject or a level in an untreated subject.

43. The method of any one of claims 40-42, wherein the reducing is detected at least by day 14 following the administering.

44. A method for treating autoimmune disease in a subject in need thereof comprising administering a Tn3 scaffold comprising a CD40L-specific monomer subunit that binds CD40L to the subject when a level of a biomarker is increased in a sample of the subject as compared to a healthy baseline level, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16.

45. The method of claim 44, wherein the biomarker is expressed on the surface of a cell.

46. The method of claim 44, wherein the biomarker is an antibody.

47. The method of claim 44, wherein the biomarker is secreted by a cell.

48. The method of any one of claims 44-47, wherein the biomarker is selected from the group consisting of: Ki67+CD27+ memory cell, CD27 high CD38 high plasmablast, CD11c high atypical memory cell, CXCL13, RF autoantibody, and any combination thereof.

49. The method of any one of claims 44-48, wherein the biomarker is selected from the group consisting of TNFRSF13B, BAFF, CXCL10, CXCL12, TL6, CCL21, CXCL9, IL12B, TNFRSF4, LAMP3, CD22, CD79B, SLAMF7, IL10, TNFRSF8, CXCL11, CCL19, TNFRSF9, TNFRSF6B, NOS3, CD72, ADA2, TL2RA, CD27, PDCD1, IL12A, IL12B, LAG3, ADGRE1, TIGIT, FCAMR, and CD80, and any combination thereof.

50. The method of any one of claims 44-49, wherein the autoimmune disease is selected from the group consisting of: systemic lupus erythematosus (SLE), lupus nephritis, cutaneous lupus erythematosus (CLE), Sjögren's syndrome, dermatomyositis, myositis, sclerosis, diabetes, Hashimoto's disease, autoimmune adrenal insufficiency, anemia, rheumatic carditis, psoriasis, arthritis, inflammation, chronic rheumatism, vitiligo, alopecia areata, hidradenitis suppurativa, celiac disease, graft versus host disease (GVHD), myocardial infarction, and a Type-1 interferonopathy.

51. The method of claim 50, wherein the arthritis is rheumatoid arthritis.

52. The method of any one of claims 44-51, wherein the administering is continued until the level of the biomarker reverts to the healthy baseline.

53. The method of any one of claims 44-52, wherein the level of the biomarker is analyzed routinely.

54. The method of any one of claims 44-53, wherein the Tn3 scaffold comprises SEQ ID NO: 1.

55. The method of any one of claims 44-54, wherein the Tn3 scaffold is administered at a dose of about 1500 mg, about 2500 mg, or about 3000 mg.

56. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the treatment of the subject with the Tn3 scaffold decreases the level of B cells selected from the group consisting of: Ki67+CD27+ memory cells, CD27 high CD38 high plasmablasts, and CD11c high atypical memory cells, as compared to a baseline level of the subject or a level in an untreated subject.

57. The method of claim 56, wherein the Tn3 scaffold comprises SEQ ID NO: 1.

58. The method of any one of claims 56-57, wherein the Tn3 scaffold is administered at a dose of about 1500 mg, about 2500 mg, or about 3000 mg.

59. The method of any one of claims 56-58, wherein the level of B cells is reduced by at least about 1-fold, 5-fold, 10-fold, 30-fold, 50-fold, 75-fold, 100-fold, 150-fold, or 200-fold.

60. A method for treating Sjögren's syndrome (SS) in a subject in need thereof comprising administering a Tn3 scaffold comprising a CD40L-specific monomer subunit that binds CD40L to the subject, wherein the monomer subunit comprises seven beta strands designated A, B, C, D, E, F, and G, and six loop regions designated AB, BC, CD, DE, EF, and FG, wherein the AB loop comprises SEQ ID NO: 11, the BC loop comprises SEQ ID NO: 12, the CD loop comprises SEQ ID NO: 13, the DE loop comprises SEQ ID NO: 14, the EF loop comprises SEQ ID NO: 15, and the FG loop SEQ ID NO: 16, and wherein the Tn3 scaffold is administered to a subject having increased levels of T cells selected from a subset of the group consisting of: Ki67+/CD3+, Ki67+/CD4+ cells, Ki67+/CD3+/CD4+ cells, Ki67+/CD3+/CD4+/CD45RA−/CCRT7+ cells, Ki67+/CD3+/CD4+/CD45RA−/CCR7− cells, Ki67+/CD3+/CD4+/CXCR5+/ICOS+ cells, Ki67+/CD3+/CD8+ cells, Ki67+/CD3+/CD8+/CD45RA−/CCR7+ cells, Ki67+/CD3+/CD8+/CD45RA/CCR7− cells, and Ki67+/CD3+/CD8+/CXCR5+ cells, and CXCR5+/ICOS+/CD3+/CD4+ cells, as compared to a baseline level of the subject or a level in an untreated subject.

61. The method of claim 60, wherein the Tn3 scaffold comprises SEQ ID NO: 1.

62. The method of any one of claims 60-61, wherein the Tn3 scaffold is administered at a dose of about 1500 mg, about 2500 mg, or about 3000 mg.

63. The method of any one of claims 60-62, wherein the level of T cells is reduced by at least about 1-fold, 5-fold, 10-fold, 30-fold, 50-fold, 75-fold, 100-fold, 150-fold, or 200-fold.


SEQUENCE LISTING (25 sequences)

SEQ ID NO: 1 - protein, 785 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTTGGGGGGGGGGGGGGGRLDAPSQIEVKDVTDTTALITWSDDFGEYVWCELTYGIKDVPGDRTTIDLWYHHAHYSIGNLKPDTEYEVSLICRSGDMSSNPAKETFTTGGGGGGGGGGDAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 2 - protein, 83 aa, synthetic construct
IEVKDVTDTTALITWSDDFGEYVWCELTYGIKDVPGDRTTIDLWYHHAHYSIGNLKPDTEYEVSLICRRGDMSSNPAKETFTT

SEQ ID NO: 3 - protein, 83 aa, synthetic construct
IEVKDVTDTTALITWSDDFGEYVWCELTYGIKDVPGDRTTIDLWYHHAHYSIGNLKPDTEYEVSLICRSGDMSSNPAKETFTT

SEQ ID NO: 4 - protein, 585 aa, synthetic construct
DAHKSEVAHRFKDLGEENFKALVLIAFAQYLQQSPFEDHVKLVNEVTEFAKTCVADESAENCDKSLHTLFGDKLCTVATLRETYGEMADCCAKQEPERNECFLQHKDDNPNLPRLVRPEVDVMCTAFHDNEETFLKKYLYEIARRHPYFYAPELLFFAKRYKAAFTECCQAADKAACLLPKLDELRDEGKASSAKQRLKCASLQKFGERAFKAWAVARLSQRFPKAEFAEVSKLVTDLTKVHTECCHGDLLECADDRADLAKYICENQDSISSKLKECCEKPLLEKSHCIAEVENDEMPADLPSLAADFVESKDVCKNYAEAKDVFLGMFLYEYARRHPDYSVVLLLRLAKTYETTLEKCCAAADPHECYAKVFDEFKPLVEEPQNLIKQNCELFEQLGEYKFQNALLVRYTKKVPQVSTPTLVEVSRNLGKVGSKCCKHPEAKRMPCAEDYLSVVLNQLCVLHEKTPVSDRVTKCCTESLVNRRPCFSALEVDETYVPKEFNAETFTFHADICTLSEKERQIKKQTALVELVKHKPKATKEQLKAVMDDFAAFVEKCCKADDKETCFAEEGKKLVAASQAALGL

SEQ ID NO: 5 - skipped

SEQ ID NO: 6 - protein, 5 aa, synthetic construct
ALITW

SEQ ID NO: 7 - protein, 10 aa, synthetic construct
  REGION 1..10: note = linker
GGGGSGGGGS

SEQ ID NO: 8 - protein, 15 aa, synthetic construct
  REGION 1..15: note = linker
GGGGSGGGGSGGGGS

SEQ ID NO: 9 - protein, 10 aa, synthetic construct
  REGION 1..10: note = linker
GGGGGGGGGG

SEQ ID NO: 10 - protein, 15 aa, synthetic construct
  REGION 1..15: note = linker
GGGGGGGGGGGGGGG

SEQ ID NO: 11 - protein, 7 aa, synthetic construct
KDVTDTT

SEQ ID NO: 12 - protein, 9 aa, synthetic construct
SDDFGEYVW

SEQ ID NO: 13 - protein, 7 aa, synthetic construct
KDVPGDR

SEQ ID NO: 14 - protein, 6 aa, synthetic construct
WYHHAH

SEQ ID NO: 15 - protein, 8 aa, synthetic construct
GNLKPDTE

SEQ ID NO: 16 - protein, 10 aa, synthetic construct
RSGDMSSNPA

SEQ ID NO: 17 - protein, 7 aa, synthetic construct
CELTYGI

SEQ ID NO: 18 - protein, 5 aa, synthetic construct
TTIDL

SEQ ID NO: 19 - skipped

SEQ ID NO: 20 - protein, 7 aa, synthetic construct
YEVSLIC

SEQ ID NO: 21 - protein, 6 aa, synthetic construct
KETFTT

SEQ ID NO: 22 - protein, 85 aa, synthetic construct
SQIEVKDVTDTTALITWSDDFGEYVWCELTYGIKDVPGDRTTIDLWYHHAHYSIGNLKPDTEYEVSLICRSGDMSSNPAKETFTT

SEQ ID NO: 23 - protein, 10 aa, synthetic construct
RLDAPSQIEV

SEQ ID NO: 24 - protein, 5 aa, synthetic construct
SQIEV

SEQ ID NO: 25 - protein, 90 aa, synthetic construct
RLDAPSQIEVKDVTDTTALITWSDDFGEYVWCELTYGIKDVPGDRTTIDLWYHHAHYSIGNLKPDTEYEVSLICRSGDMSSNPAKETFTT